SEQUENCE LISTING (6 sequences)

SEQ ID NO: 1 - protein, 10 aa, Artificial sequence
  Nuclear localization signal-containing peptide
CTPPKKKRKV

SEQ ID NO: 2 - protein, 7 aa, Artificial sequence
  Nuclear localization signal peptide
PKKKRKV

SEQ ID NO: 3 - protein, 10 aa, Artificial sequence
  Reverse-nuclear localization signal-containing peptide
CTPVKRKKKP

SEQ ID NO: 4 - protein, 7 aa, Artificial sequence
  Reverse-nuclear localization signal peptide
VKRKKKP

SEQ ID NO: 5 - protein, 12 aa, Artificial sequence
  M9 motif consensus sequence
XXXXXSXXGXXX

SEQ ID NO: 6 - protein, 12 aa, Artificial sequence
  M9 nuclear localization signal (NLS) sequence derived from hnRNP A1
YNNQSSNFGPMK

(19) United States
(12) Patent Application Publication
(10) Pub. No.: US 2006/0239988 A1
(43) Pub. Date: Oct. 26, 2006

(54) Neuronal regeneration and compound administration methods

(76) Inventors: Michael Fainzilber, Rechovot (IL); Shlomit Hanz, Tikva (IL); Eran Perlson, Shmuel (IL)

Correspondence Address: Martin Moynihan;Prtsi Inc, P O Box 16446, Arlington, VA, 22215, US

(21) Appl. No.: 10/559,871

(22) PCT Filed: Jun. 9, 2004
(86) PCT No.: PCT/IL04/00492

§ 371 Date: May 2, 2006

Related U.S. Application Data
(60) Provisional application No. 60/476,912, filed on Jun. 9, 2003.

(51) Int. Cl. A61K 38/48; C07H 21/04; C12P 21/06; C12N 5/08; C07K 14/705

(52) U.S. Cl. 424/94.5; 435/69.1; 435/320.1; 435/368; 530/350; 536/23.5

(57) ABSTRACT

A method of modulating growth of an axon is disclosed. The method comprises regulating importin mediated retrograde transport in the axon.

### FIELD AND BACKGROUND OF THE INVENTION

The present invention relates to methods of regulating growth of a neuron, and to methods of delivering compounds to neurons. In particular, embodiments of the present invention relate to methods of delivering therapeutic or diagnostic compounds to neurons via retrograde transport, and to methods of regulating regenerative growth of axons.

Diseases associated with the nervous system include neurodegenerative diseases, malignancies, infectious diseases, stroke and physical injury, and developmental diseases of the brain. Such nervous system associated diseases represent numerous highly debilitating and/or lethal diseases affecting large numbers of individuals, for which no satisfactory treatment or diagnostic method is available. For example, in the United States, approximately 12,000 people each year suffer some form of spinal cord injury, with over 200,000 people chronically paralyzed as a consequence of such injury. The pathology of many of these diseases is associated with disregulated retrograde transport dependent cellular physiological processes, such as growth, survival, and differentiation (for example, refer to: Heerssen H M. and Segal R A., 2002. Trends Neurosci. 25:160-5; Friedman W J. and Greene L A., 1999. Exp Cell Res. 253:131-42; Thoenen H., 1995. Science 270:593-8; Korsching S., 1993. J Neurosci. 13:2739-48). For example, breakdown or impairment of axonal transport mechanisms has been implicated in the initiation or progression of a number of neurodegenerative diseases (Gunawardena, S., and L. S. Goldstein. 2004. J Neurobiol. 58:258-71; Salehi, A. et al., 2003. Trends Neurosci. 26:73-80).

Neuronal regeneration is a transcription/translation dependent process wherein the neuronal cell body changes patterns of macromolecular synthesis in response to an injury event in the axon (for example, refer to: Caroni C., 1998. Essays Biochem. 33:53-64; Goldberg J L. and Barres A B., 2000. Annu Rev Neurosci 23:579-612; Snider W D. et al., 2002. Neuron 35:13-6; Plunet et al., 2002. J Neurosci Res 68, 1-6). Axonal lesions may induce up-regulation of transcription and/or translation of transcription factors, cytoskeletal proteins, cell adhesion and axon guidance molecules, and trophic factors and their receptors (Goldberg, J. L. 2003. Genes Dev. 17:941-58). Studies in molluscan models have suggested that these injury signals include axoplasmic proteins, which are activated at the injury site, and conveyed by retrograde transport to the cell body (Ambron and Walters, 1996. Molecular Neurobiology 13, 61-79; Perlson et al., 2004. Mol Cell Proteomics 3, 510-520). Mammalian peripheral and invertebrate central nerves in particular are capable of functional regeneration, in part due to intrinsic mechanisms activated in the neuronal cell body (Goldberg, J. L. et al., 2002. Science 296, 1860-1864; Neumann, S., and Woolf, C. J., 1999. Neuron 23, 83-91; Rossi et al., 2001. Restor Neurol Neurosci 19, 85-94). Thus, the cell body of a lesioned neuron must receive accurate and timely information on the site and extent of axonal damage, in order to allow such transcriptional and translational responses. The extremely long axonal and dendritic processes extended from neuronal cell bodies require transport mechanisms to move macromolecules and metabolites to and from the cell body and neurite terminals. Such mechanisms, which include both anterograde and retrograde transport mechanisms, are mediated by large motor protein complexes centered around kinesins and dynein, respectively (Vallee, R. B. et al., 2004. J Neurobiol. 58:189-200). In particular, retrograde transport of neurotrophic signals from axon terminals to the cell soma is thought to play a critical role in neuronal survival, maintenance, outgrowth and plasticity (Ginty, D. D., and R. A. Segal., 2002. Curr Opin Neurobiol. 12:268-74), and mechanistic explanations for how such signals might interact with the dynein complex have recently started to emerge (Yano, H., and M. V. Chao. 2004. J Neurobiol. 58:244-57).

Thus, retrograde signaling from axonal lesion sites has been hypothesized to play a role in activating cell body responses to injury (Ambron, R. T., and E. T. Walters. 1996. Mol Neurobiol. 13:61-79; Perlson, E. et al., 2004. J Neurobiol. 58:287-94). It has been demonstrated that the regenerative capacity of dorsal root ganglia (DRG) neurons is enhanced in a transcription-dependent manner if peripheral processes are subjected to a conditioning lesion several days prior to the actual axotomy (Smith, D. S., and J. H. Skene. 1997. J Neurosci. 17:646-58). The initial signal from the injury site to the cell body is thought to be a rapid depolarization-induced burst of action potentials. Hours to days after this response, changes in at least two types of retrograde signals impinge on the cell soma, including interruption of the arrival of macromolecules normally trafficked from terminals, and the appearance of new axoplasmic proteins “activated” by modification at the injury site. Microinjection of retrogradely concentrated axoplasm from lesioned Aplysia nerves into neurons in culture has been shown to lead to uptake of microinjected proteins into the nucleus concomitantly with growth and survival responses in neuronal cell bodies (Ambron, R. T. et al., 1996. J Neurosci. 16:7469-77; Zhang, X. P., and R. T. Ambron. 2000. J Neurobiol. 45:84-94; Ambron R T. and Walters E T., 1996. Molecular Neurobiology 13:61-79). Retrograde transport of lesion axoplasm components has been suggested to be dependent on nuclear localization signals (NLS) in the proteins (Ambron and Walters, 1996. Molecular Neurobiology 13, 61-79; Schmied, R., and R. T. Ambron. 1997. J Neurobiol. 33:151-60), in addition to the well known role of NLS sequences in mediating nuclear import, as shown via capacity of an SV40 nuclear localization signal (NLS) to target retrograde transport and nuclear uptake of heterologous proteins after microinjection thereof into axons. Nuclear localization signals are generally short stretches of 6-8 amino acid residues that enable active transport of proteins through nuclear pore complexes in the membrane of the nucleus. Retrograde axonal transport of an endogenous NLS-bearing protein has also been reported in mammals (Wellmann H. et al., 2001. J Biol. Chem. 276:11821-9). Until recently, axoplasmic proteins containing NLSs were postulated to act as retrograde injury signals in lesioned nerve, but the molecular mechanisms mediating such signaling remained unknown (Ambron, R T. and Walters, E T., 1996. Molecular Neurobiology 13:61-79; Schmied, R. and Ambron, R T., 1997. J Neurobiol. 33:151-60). It is now known that active nuclear transport is mediated by binding of the NLS to soluble transport factors that belong to the importin (also known as karyopherin) families of proteins (Weis, K 2003. Cell. 112:441-51).

The critical triggering event for the mechanism outlined above is local synthesis of importin-beta at the lesion site in axons. Axonal translation is thought to occur for a wide range of gene products (Giuditta, A. et al., 2002. Trends Neurosci. 25:400-4), contributing to processes and systems ranging from growth cone tuning in retinal ganglion cells (Campbell, D. S., and C. E. Holt. 2001. Neuron. 32:1013-26), localized regulation of axonal guidance at the midline (Brittis, P. A. et al., 2002. Cell. 110:223-35), and regeneration of adult sensory neurons (Zheng, J. Q. et al., 2001. J Neurosci. 21:9291-303). The common theme in most previous studies has been local synthesis for local use, thus for example it has been shown that local synthesis of a guidance receptor at a point wherein the axons should change their path by signaling of the cognate ligand (Brittis, P. A. et al., 2002. Cell. 110:223-35), and it has been suggested that intra-axonal translation of cytoskeletal components is required for sustaining growth cones in regenerating axons (Zheng, J. Q. et al., 2001. J Neurosci. 21:9291-303). In contrast, local synthesis of importin-beta underlies formation of a long distance signal, thus providing a mechanism by which the axon can influence or regulate distant signaling events. These data therefore extend the potential temporal and spatial influence of local axonal translation, suggesting that maintenance of latent signaling complexes in the form of specific axonal transcripts provides a versatile mechanism for regulated long range signaling by locally translating critical components of the system.

Nuclear import of proteins is also mediated by binding of the NLS of nuclear-targeted proteins to members of the importin/karyopherin family, soluble transport factors mediating translocation of substrates through the nuclear pore complex (Gorlich D. and Kutay U., 1999. Annu Rev Cell Dev Biol. 15:607-60; Chook Y. and Blobel G., 2001. Curr Opin Struct Biol. 11:703-15). For example, it has been demonstrated that the classical SV40-type NLS binds with low affinity to importin-alpha, and with high affinity to importin-alpha/-beta heterodimers which conveys transport of the complex through the nuclear pore (FIG. 1; Jans D A. et al., 2000. Bioessays 22:532-44; Kohler M. et al., 1999. Mol Cell Biol. 19:7782-91). The formation and dissociation of the importin-alpha/-beta complex during such processes is thought to be regulated by the small GTPase Ran and other nuclear and cytoplasmic proteins. The nuclear import function of importins has led to the implicit assumption that they should be found in a perinuclear distribution in cells, although they may also modulate interactions with cytoplasmic microtubules to funnel nuclear-targeted proteins to the nuclear pore (Smith H M. and Raikhel N V., 1998. Plant Cell 10:1791-9; Lam M H. et al., 2002. Mol Endocrinol. 16:390-401; Mavlyutov T. A. et al, 2002. Traffic 3:630-40).

One of the central obstacles preventing treatment or effective treatment of nervous system associated diseases, such as diseases of the central nervous system (CNS) whose pathogenesis is associated with disregulated neuronal growth is the lack of an effective approach, despite intensive research, for promoting significant regeneration of CNS neurons/neuronal tissues which, following injury, have insufficient or no capacity to regenerate, in contrast to peripheral nervous system neurons. In the case of nervous system associated diseases treatable, or theoretically treatable, by surgical intervention, such as diseases affecting brain cells/tissues, such as brain tumors, obstacles to treatment or effective treatment include surgical inaccessibility, or insufficient surgical accessibility, to affected tissues/cells (Zhu, Y. and Parada, L F., 2002. Nat Rev Cancer 2:616-26), diffuse and/or disseminated localization of affected cells/tissues which often does not allow tumor excision without irreversible damage to the patient. The impenetrability of the blood-brain barrier to most drugs further represents a significant obstacle to treatment of brain diseases via conventional pharmacological/chemotherapeutic therapies (for example, refer to Pardridge, W M., 2002. Nat Rev Drug Discov. 1:131-9). So far most efforts of delivering compounds to brain tissues have focused on finding ways of breaching or bypassing the blood brain barrier. Various approaches aimed at repairing CNS damage have focused on the role of the environment of the injured nerve in inhibiting or encouraging repair (Morgenstern, D. A. et al., 2002. Prog Brain Res. 137:313-32; Raisman, G. 2004. Nat Rev Neurosci. 5:157-61; Spencer, T. et al., 2003. Curr Opin Neurobiol. 13:133-9). Nonetheless, it is increasingly becoming clear that effective repair and regeneration is dependent on both environmental factors and on mobilization of intrinsic mechanisms within neurons (Gianola, S., and F. Rossi. 2002. Exp Neurol. 176:25-40; Goldberg, J. L. 2003. Genes Dev. 17:941-58; Snider, W. D. et al., 2002. Neuron. 35:13-6). A very recent study has demonstrated this point by showing that inhibiting Nogo receptor signaling in retinal ganglion cells increased axon regeneration only in cells already sensitized for growth by activation of other pathways (Fischer, D. et al., 2004. J Neurosci. 24:1646-51).

Thus, numerous nervous system associated diseases whose pathogenesis involves disregulated neuronal proliferation/differentiation, such as developmental diseases of the brain, are not currently treatable or are not satisfactorily treatable. Furthermore, pharmacological/chemotherapeutic treatment of nervous system associated diseases is often ineffective due to the general cellular heterogeneity and complex circuitry of the nervous system which makes it difficult to achieve effective delivery of therapeutic compounds to diseased neurons/neuronal tissues. Diagnosis of nervous system associated diseases is equally hindered as a result of such obstacles to delivering exogenous compounds to diseases neurons/neuronal tissues.

Thus, improved/novel methods of treating and diagnosing nervous system associated diseases are urgently required.

An optimal strategy for treating/diagnosing nervous system associated diseases would be to exploit/regulate neuronal retrograde transport mechanisms so enable delivery of therapeutic/diagnostic compounds to neuronal cell bodies, such as neuronal cell bodies which are difficult or impossible to access and/or localize. Methods of regulating neuronal retrograde transport mechanisms could also used for optimally modulating physiological processes of neurons, such as growth, which are retrograde transport-dependent.

Several prior art approaches have been employed or suggested for exploiting/regulating neuronal retrograde transport mechanisms for delivering exogenous compounds to neuronal cell bodies, or for regulating physiological processes such as growth in neurons.

One approach has attempted using microinjection of total protein from axoplasm of damaged axons in attempts to elicit retrograde transport-dependent growth and survival responses in injured Aplysia neurons (Zhang X P. and Ambron R T., 2000. J Neurobiol. 45:84-94).

Another approach involves attaching exogenous compounds to SV40 nuclear localization signal (NLS; Schmied R and Ambron R T., 1997. J Neurobiol. 33:151-60) or to Rel A NLS (Wellmann H. et al., 2001. J Biol Chem. 276:11821-9) in attempts to induce retrograde transport of such proteins.

A further approach involves regulating signaling via the ERK5 mitogen-activated protein kinase (MAPK) pathway (Watson F L. et al., 2001. Nat Neurosci. 4:981-8) or via the Trk pathway (Ginty D D., and Segal R A., 2002. Curr Opin Neurobiol. 12:268-74; Miller, F D. and Kaplan D R., 2001. Neuron 32:767-70) in attempts to induce neuronal regeneration-enhancing retrograde signals.

Yet a further approach involves infecting neurons with viral vectors in order to attempt to achieve retrograde delivery of vector-encoded exogenous protein, whereby viral delivery to axon terminal fields in the hippocampus and striatum resulted in viral internalization, retrograde transport, and transgene expression in specific projection neurons in entorhinal cortex and substantia nigra (Kaspar B K. et al., 2002. Mol Ther. 5:50-6).

All of the aforementioned approaches, however, suffer from significant disadvantages, including: (i) having no demonstrated applicability for use in mammalian neurons, such as in the case of approaches demonstrated using Aplysia neurons, which are biologically significantly distinct from mammalian neurons; (ii) their being impractical or cumbersome to perform, such as in the case of approaches involving microinjection of total protein from injured axoplasm, or construction and administration of viral vectors; (iii) their having no demonstrated capacity for specifically inducing retrograde transport of exogenous compounds, such as in the case of approaches involving regulation of Trk signaling; (iv) their being hazardous, for example as a consequence of using undefined, and hence potentially harmful, populations of molecules, such as in the case of approaches involving microinjection of total protein from injured axoplasm, or as a consequence of employing viral infection (Berry, M. et al., 2001. Curr Opin Mol Ther. 3:338-49); and/or (v) their having no demonstrated capacity for regulating neuronal growth, such as in the case of approaches involving the use of NLS-tagged proteins, regulation of Trk signaling, viral infection, or regulation of ERK5 signaling.

Thus, all prior art approaches have failed to provide an adequate solution for exploiting/regulating neuronal retrograde transport mechanisms.

There is thus a widely recognized need for, and it would be highly advantageous to have, a method devoid of the above limitation.

### SUMMARY OF THE INVENTION

According to one aspect of the present invention there is provided a method of inducing retrograde transport of an exogenous compound in an axon, the method comprising: (a) increasing in the axon an activity and/or a level of a molecule participating in importin mediated retrograde transport; and (b) administering the exogenous compound to the axon, the exogenous compound being capable of directly or indirectly associating with the molecule participating in importin mediated retrograde transport, thereby inducing retrograde transport of the exogenous compound in the axon.

According to further features in preferred embodiments of the invention described below, step (b) is effected prior to, concomitantly with, or following step (a).

According to another aspect of the present invention there is provided a method of modulating growth of an axon, the method comprising regulating importin mediated retrograde transport in the axon, thereby modulating growth of the axon.

According to further features in preferred embodiments of the invention described below, regulating importin mediated retrograde transport in the axon is effected by altering in the axon an activity and/or a level of a molecule participating in importin mediated retrograde transport.

According to still further features in the described preferred embodiments, regulating importin mediated retrograde transport in the axon is up-regulating importin mediated retrograde transport in the axon, and up-regulating importin mediated retrograde transport in the axon is effected by increasing in the axon an activity and/or a level of the molecule participating in importin mediated retrograde transport.

According to still further features in the described preferred embodiments, increasing in the axon the activity and/or level of the molecule participating in importin mediated retrograde transport is effected by administering to the axon at least one agent selected from the group consisting of: (i) an exogenous polynucleotide sequence designed and constructed to express at least a functional portion of the molecule participating in importin mediated retrograde transport; (ii) a molecule capable of activating the molecule participating in importin mediated retrograde transport; and (iii) the at least a functional portion of the molecule participating in importin mediated retrograde transport.

According to still further features in the described preferred embodiments, regulating importin mediated retrograde transport in the axon is down-regulating importin mediated retrograde transport in the axon, and down-regulating importin mediated retrograde transport in the axon is effected by decreasing in the axon the activity and/or level of the molecule participating in importin mediated retrograde transport.

According to still further features in the described preferred embodiments, decreasing in the axon the activity and/or level of the molecule participating in importin mediated retrograde transport is effected by administering to the axon at least one agent selected from the group consisting of: (a) a molecule capable of binding the molecule participating in importin mediated retrograde transport; (b) an siRNA molecule capable of inducing degradation of an RNA encoding the molecule participating in importin mediated retrograde transport; (c) an antisense polynucleotide capable of hybridizing with an mRNA encoding the molecule participating in importin mediated retrograde transport; (d) a ribozyme capable of cleaving an mRNA encoding the molecule participating in importin mediated retrograde transport; and (e) a molecule capable of inhibiting ligand-binding of the molecule participating in importin mediated retrograde transport.

According to yet another aspect of the present invention there is provided a composition-of-matter comprising a compound associated with a molecule participating in importin mediated retrograde transport in an axon, wherein the compound is capable of regulating in a cell a physiological process selected from the group consisting of growth, retrograde transport, survival, and differentiation.

According to further features in preferred embodiments of the invention described below, the cell is a neuron or a neuron-associated cell.

According to still further features in the described preferred embodiments, the neuron is an injured neuron.

According to still another aspect of the present invention there is provided a polynucleotide encoding a chimeric polypeptide comprising at least a portion of a molecule participating in importin mediated retrograde transport in an axon, the at least a portion of a molecule capable of regulating in a cell a physiological process selected from the group consisting of growth, retrograde transport, survival, and differentiation.

According to further features in preferred embodiments of the invention described below, the molecule participating in importin mediated retrograde transport is participating in importin-beta mediated retrograde transport, transportin mediated retrograde transport or importin-alpha mediated retrograde transport.

According to still further features in the described preferred embodiments, the molecule participating in importin-beta mediated retrograde transport is participating in importin-beta1 mediated retrograde transport.

According to still further features in the described preferred embodiments, the molecule participating in importin-alpha mediated retrograde transport is participating in importin-alpha4 mediated retrograde transport.

According to still further features in the described preferred embodiments, the molecule participating in importin mediated retrograde transport is selected from the group consisting of an importin, an intermediate filament protein, a molecule including a nuclear localization signal, and an ERK.

According to still further features in the described preferred embodiments, the importin is importin-alpha, transportin or importin-beta.

According to still further features in the described preferred embodiments, the importin-beta is importin-beta1.

According to still further features in the described preferred embodiments, the importin-alpha is importin-alpha4.

According to still further features in the described preferred embodiments, the intermediate filament protein is a type III intermediate filament protein.

According to still further features in the described preferred embodiments, the type III intermediate filament protein is vimentin or peripherin.

According to still further features in the described preferred embodiments, the nuclear localization signal includes an amino acid sequence set forth in SEQ ID NO: 2 or 5.

According to still further features in the described preferred embodiments, the ERK is selected from the group consisting of ERK1, ERK2 and a phosphorylated ERK.

According to still further features in the described preferred embodiments, axon is an injured axon.

According to a further aspect of the present invention there is provided a nucleic acid construct including the polynucleotide.

According to a yet a further aspect of the present invention there is provided a host cell transformed with the nucleic acid construct.

According to further features in preferred embodiments of the invention described below, the cell is a neuron.

The present invention successfully addresses the shortcomings of the presently known configurations by providing: (i) a method of inducing retrograde transport of an exogenous compound in an axon; (ii) a method of modulating growth of an axon; (iii) a composition-of-matter comprising a compound associated with a molecule participating in importin mediated retrograde transport in an axon, wherein the compound is capable of regulating in a cell a physiological process selected from the group consisting of growth, retrograde transport, survival, and differentiation; (iv) a polynucleotide encoding a chimeric polypeptide comprising at least a portion of a molecule participating in importin mediated retrograde transport in an axon, the at least a portion of the molecule being fused to an amino acid sequence capable of regulating in a cell a physiological process selected from the group consisting of growth, retrograde transport, survival, and differentiation; (v) a nucleic acid construct including such a polynucleotide; and (vi) a host cell transformed with such a nucleic acid construct.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram depicting the nuclear transport cycle. Importin-alpha and importin-beta form a heterodimer in cytoplasm that creates a high affinity binding site for NLS in cargo proteins. The resulting complex than docks via importin-beta to the cytoplasmic side of the nuclear pore complex and is translocated into the nucleus where it undergoes RanGTP mediated disassembly and release of the cargo protein. The importins are exported back to the cytoplasm, and undergo RanGDP enhanced reformation of the importin-alpha/-beta heterodimer.

FIG. 2a is a Western immunoblotting analysis photograph depicting the appearance of importin-beta protein in lesioned sciatic nerve axoplasm 6 hours post-lesion (Injured), but not in axoplasm of non-injured sciatic nerve (Control). The presence of importin-alpha1, -alpha3, and -alpha7 protein but not of importin-alpha2 or -alpha4 protein was detected in both non-injured and injured axoplasm.

FIGS. 2b-c are fluorescence photomicrographs depicting specific axonal co-localization of importin-alpha4 in non-injured sciatic nerve cross-sections with the axonal marker NF—H, but not with the myelin sheath marker MAG, respectively. Cells were fluorescently co-immunostained with anti importin-alpha4 antibody and either anti NF—H antibody or anti MAG antibody, respectively. In FIG. 2b, staining specific to NF—H(NF—H), importin-alpha4 (α3), or both (Overlay) are shown. In FIG. 2c, staining specific to MAG (MAG), importin-alpha4 (α3), or both (Overlay) are shown. An expanded view of the overlay is shown in the right-hand side panel.

FIG. 2d is a fluorescence photomicrograph depicting specific axonal localization of importin-beta in lesioned sciatic nerve cross-sections co-immunostained with antibodies specific for importin-beta and the axonal marker NF—H. Views depicting staining specific to importin-beta (β), NF—H (NF—H), or both importin-beta and NF—H (Overlay) are shown. An expanded view of the overlay is shown in the right-hand side panel.

FIGS. 2e-f are fluorescence photomicrographs depicting the presence of both importin-beta and importin-alpha4 in cell bodies, axons and dendrites of growing adult dorsal root ganglion (DRG; FIG. 2e) and hippocampal neurons (FIG. 2f). Dorsal root ganglion and hippocampal neurons were cultured for 3 and 7 days, respectively, and co-immunostained with antibody specific for importin-alpha4 and antibody specific for importin-beta. Micrographs were taken at ×60 magnification. Staining specific to importin-beta (β), importin-alpha4 (α), or both (Overlay) are shown. Expanded views of the overlays are shown in the right-hand side panels.

FIGS. 3a-b are bar graphs depicting steadily increasing levels of importin-beta (β) protein over time but relatively stable levels of importin-alpha4 (α3) protein, respectively, following lesioning in sciatic nerve axoplasm. Samples of 40 micrograms of total axoplasmic protein were analyzed by Western immunoblotting analysis and levels of importin were quantified at the indicated time-points.

FIG. 3c is an electrophoretic gel autoradiograph depicting de-novo synthesis of importin-beta protein in sciatic nerve axoplasm following lesioning. Lesioned sciatic nerve segments were incubated for various time periods in Met/Cys-deficient DMEM medium supplemented with 1 mCi/ml of [35]S-Met/Cys, in the presence or absence of 10 micrograms/ml of the protein synthesis inhibitor cycloheximide (CHX) or 5 micrograms/ml of the RNA transcription inhibitor actinomycin-D (ActD). Axoplasm from incubated nerve segments was immunoprecipitated with anti importin-beta antibody. Note that radioactive importin-beta protein was not formed in the presence of CHX, while addition of ActD merely reduced the levels of the radioactive protein accumulation. Thus, there is de-novo translation of importin-beta from preexisting mRNA in the nerve.

FIG. 3d is a series of photomicrographs depicting the presence of importin-beta mRNA lesioned sciatic nerve. Nerve axoplasm was analyzed by in-situ hybridization. Four serial 7-micron sections are shown, with the positive axon indicated by the arrow. Numbers “1” and “2” identify neighboring axons. Note that the hybridization signal is localized in two consecutive sections, suggesting localized longitudinal concentrations of the transcript within axons.

FIG. 4a is a Western immunoblotting analysis photograph depicting formation of a complex comprising importin-alpha4 and NLS peptide (NLS) but not reverse-NLS peptide (Reverse) in axoplasm of lesioned sciatic nerve. Biotinylated NLS peptide or biotinylated reverse-NLS peptide (100 micromolar) was incubated with 0.5 mg of sciatic nerve axoplasm overnight at 4 degrees centigrade, followed by pull-down with streptavidin dynabeads. Proteins were eluted with 0.1% trifluoroacetic acid and analyzed via Western immunoblotting using an anti importin-alpha4 antibody probe.

FIG. 4b is a Western immunoblotting analysis photograph depicting formation of a complex comprising importin-beta and NLS peptide in lesioned but not control sciatic nerve axoplasm, and 3-fold higher levels of association of importin-alpha4 with NLS-peptide in lesioned (Injured) versus control sciatic nerve axoplasm. Biotinylated NLS (NLS) peptide or biotinylated reverse-NLS (Reverse) peptide (90 micromolar was incubated with 0.5 mg of sciatic nerve axoplasm overnight at 4 degrees centigrade, followed by pull-down with streptavidin dynabeads. Proteins were eluted with 0.1% trifluoroacetic acid and subjected to Western immunoblotting analysis using anti importin-alpha4 (α3) or anti importin-beta (β) antibody probes.

FIG. 4c is a Western immunoblotting analysis photograph depicting formation of a complex comprising importin-beta and the retrograde motor protein dynein in axoplasm of lesioned but not control sciatic nerve, and formation of a complex comprising importin-alpha4 and dynein in axoplasm of both control and lesioned sciatic nerve. Proteins were analyzed via Western immunoblotting analysis using anti importin-alpha4 (α3) or anti importin-beta (β) antibody.

FIG. 4d is a Western immunoblotting analysis photograph depicting formation of a complex comprising dynein and NLS peptide in lesioned but not control sciatic nerve axoplasm. Proteins were analyzed via Western immunoblotting analysis using anti dynein antibody for detection. The association of dynein with NLS was analyzed via NLS pull-down assay of dynein from lesioned (injured) and control sciatic nerve axoplasm. Note that both control and injured nerve axoplasm contained comparable levels of dynein, indicating that a complex mediating NLS interaction with dynein is available only in injured axons.

FIG. 4e is a pair of fluorescence photomicrographs depicting retrograde trafficking of NLS peptide in injured rat sciatic nerve in-vivo. Biotinylated NLS peptide was microinjected into the nerve at the site indicated by the white arrows, while concomitantly a crush lesion was applied to the nerve adjacent to the injection site. Six hours later the nerve was harvested and processed for sectioning. Longitudinal reconstructions of representative nerves at the injection time (0 hr) and 6 hours later (6 hr) are shown. The biotinylated NLS peptide and the axonal marker NFH were visualized as green and red immunofluorescent signals, respectively.

FIG. 5a is a set of fluorescence photomicrographs depicting impairment of regenerative outgrowth of adult DRG neurons by NLS peptide. Neurons were triturated, and during the trituration NLS peptide, reverse-NLS peptide, or no peptide (NLS, Reverse, or Control, respectively) was added to the neurons. Following trituration, the cells were cultured, stained with anti NF—H antibody, and the appearance of the cultures was recorded by fluorescence photomicrography (×20 magnification) at 24-, 48-, and 72-hour time points. As a negative control, neurons were triturated without peptide.

FIGS. 5b-c are bar graphs depicting significant reductions in the percentage of regenerative sprouting and significant reductions in regenerative neurite growth, respectively, in populations of neurons having cell bodies at least 12 microns in diameter and of neurons having cell bodies less than 12 microns in diameter treated with NLS peptide relative to reverse-NLS peptide. Neurons were triturated, and during the trituration NLS peptide or reverse-NLS peptide was added to the neurons. Following trituration, the cells were cultured and neurite outgrowth of NFH-positive neurons and percent sprouting were measured after 48 hours in culture. * indicates p<0.05, ** indicates p<0.01.

FIG. 6 is a schematic model depicting the formation of an importins-targeted retrograde injury-signaling complex. Importin-alpha protein is constitutively associated with the retrograde motor dynein (D) in axons, whereas importin-beta is normally present only as mRNA (upper panel). Upon lesion, local translation of importin-beta to protein induces formation of the importin heterodimer, thus creating a high affinity NLS-binding site associated with dynein. Concomitant modification of NLS-bearing signaling proteins in axoplasm creates a signaling cargo that binds to the complex, thus accessing the retrograde transport pathway (middle and lower panels). Previously described modifications of signaling proteins in axoplasm (for example, refer to Ginty D D., and Segal R A., 2002. Curr Opin Neurobiol. 12:268-74) may concomitantly create signaling cargos that bind to the NLS binding sites on the importins, thus accessing the retrograde transport pathway.

FIG. 7 is a series of photomicrographs depicting the specific presence of the intermediate filament protein vimentin in injured axons of adult rat sciatic nerve. The left panels represent non-fluorescent light micrographs of the analyzed microscope fields. Cells were fluorescently co-immunostained for detection of the axonal marker NF—H and the intermediate filament protein vimentin (upper panel set) or for detection of the myelin sheath marker MAG and the intermediate filament protein vimentin (lower panel set). The respective overlay views of both co-immunostainings are shown in the right-hand side panels of the corresponding panel set.

FIG. 8 is a Western immunoblotting analysis depicting appearance and increasing levels of the soluble monomers of the intermediate filament proteins vimentin and peripherin in sciatic nerve axoplasm following axonal injury.

FIG. 9 is a Western immunoblotting analysis photograph depicting co-immunoprecipitation of peripherin or vimentin with dynein from injured nerve axoplasm. Injured sciatic nerve axoplasm was immunoprecipitated with anti dynein antibody, and the immunoprecipitated proteins were analyzed via Western immunoblotting using anti peripherin or anti vimentin antibody probes.

FIG. 10 is a Western immunoblotting analysis depicting retrograde transport of vimentin microinjected into sciatic nerve. 0.5 microgram aliquot of vimentin was microinjected into the nerve, and after 6 hours, axoplasm from axon segments A and B, positioned relative to the site of injection, to the cell body, and to each other as shown in the accompanying schematic diagram, was analyzed via Western immunoblotting analysis for the presence of vimentin. As a negative control, a sham injection containing no vimentin (0 microgram) was performed.

FIG. 11 is a Western immunoblotting analysis photograph depicting formation of a complex comprising vimentin and importin-beta in axoplasm of injured sciatic nerve. The association was analyzed via an in-vitro pull-down assay of vimentin with GST-conjugated importin-beta.

FIG. 12 is a Western immunoblotting analysis photograph depicting association of soluble endogenous vimentin of sciatic nerve axoplasm with importin-beta. The association was analyzed via a pull-down assay of axoplasm with (Pull down) or, as a negative control, without GST-importin-beta (Ext only).

FIG. 13 is a Western immunoblotting analysis photograph depicting association of vimentin with mitogen-activated protein kinase ERK1 and ERK2 in the non-phosphorylated (MAPK) or phosphorylated state (phospho-MAPK). Shown is a Western immunoblotting analysis of MAPK co-immunoprecipitated with vimentin.

FIGS. 14a-b depict impairment of regenerative outgrowth of DRG neurons in vimentin-null mice. FIG. 14a is series of fluorescence photomicrographs depicting reduction in regenerative outgrowth of NFH- and peripherin-positive neurons. Dorsal root ganglion neurons from vimentin-null (Vim−/−) and wild-type (w.t) mice were fluorescently immunostained with antibodies specific for NFH (anti NFh) or peripherin (anti Peripherin). FIG. 14b is a bar graph depicting reduction in neurite length following regenerative outgrowth of DRG neurons in vimentin-null mice (vim k/o) relative to wild-type mice (w.t).

FIG. 15a is a phylogenetic tree constructed using ClustalX revealing the evolutionary relatedness of mammalian vimentin and other type III intermediate filaments to Lymnaea RGP51.

FIG. 15b is a set of fluorescent photomicrographs depicting expression of vimentin in the wild-type but not in the vimentin^{-/-} neurons, and beta-Gal in vimentin^{-/-} but not in the wild-type neurons. Adult wild-type and vimentin^{-/-} DRG neurons were immunostained after two days in culture. Co-localization with the axonal marker NFH (green) showed vimentin expression in both cell body and neurites. Cell counts revealed that 64% of the wild-type NFH-positive neurons expressed vimentin, while 58% of the vimentin^{-/-} neurons expressed beta-Gal. Magnification 20×.

FIG. 15c is a set of Western blot analyses and an associated quantitative histogram depicting that vimentin and peripherin are upregulated in axoplasm after nerve injury. Adult rats were anesthetized, subjected to sciatic nerve crush, and axoplasm was obtained from nerves dissected at the indicated times (hours) post-lesion. Forty-microgram aliquots of axoplasm were analyzed by Western blot and quantified in reference to the level of vimentin and peripherin at 6 hours. Calpeptin (100 micromolar), cycloheximide (10 micrograms/ml), or actinomycin-D (5 micrograms/ml) were applied to the nerve by injection. Beta-tubulin was used to confirm equal loading. The quantification is in % of protein levels at 6 hours post-lesion (averages plus/minus standard deviation, n=3).

FIG. 16a is a set of Western blot photographs and an associated quantitative histogram depicting that vimentin interacts with the dynein/importin complex after nerve injury. Vimentin, importin-beta and importin-alpha but not peripherin were co-precipitated with dynein from axoplasm (500 microgram input per lane) at 0-6 hours post-lesion. Quantification is in reference to the protein level at 6 hours post-lesion.

FIG. 16b is a set of Western blot photographs and an associated quantitative histogram depicting that vimentin interacts with the dynein/importin complex after nerve injury. Dynein, importin-beta and importin-alpha were co-precipitated with vimentin at the indicated times (hours) post-lesion. Quantification is in reference to the protein level at 6 hours post-lesion.

FIG. 16c is a set of Western blot photographs depicting that co-precipitation of vimentin with dynein was not affected by NLS or reverse-NLS (REV) peptides at 2 micromolar concentration.

FIG. 16d is a set of Western blot photographs depicting direct interaction of importin-beta and vimentin in-vitro. GST-importin-beta (1 microgram) was incubated with vimentin (0.1 microgram) for 2 hours at 37 degrees centigrade before GST pull-down. GST alone was used as a negative control.

FIG. 16e is a set of Western blot photographs and an associated quantitative histogram of a pull-down assay depicting co-precipitation of importin-beta with vimentin but not peripherin after nerve injury. GST-Importin-beta (1 microgram) was incubated with axoplasm (500 micrograms) from 0-6 hours post lesion for 2 hours at 37 degrees centigrade. The quantification is in reference to 6 hours post-lesion (averages plus/minus standard deviation, n=3 independent experiments in each panel).

FIG. 17a is a set of Western blot photographs and an associated quantitative histogram depicting phosphorylation of ERK1 and ERK2 after axonal injury. Forty microgram aliquots of axoplasm from 0-8 hours post-lesion axon were subjected to Western blot analysis. Western blotting with a general anti-ERK antibody was used to control for equal loading. Quantification is in percentage of phosphorylated ERK (pERK) levels at 6 hours post-lesion (average plus/minus standard deviation). Experiments were repeated at least three times with similar results.

FIG. 17b is a set of Western blot photographs and an associated quantitative histogram depicting increasing association of pERK with dynein and of pERK with vimentin following axonal injury. Dynein or vimentin from 500 microgram samples of axoplasm taken at 0-6 hours post-lesion was coimmunoprecipitated with pERK. The quantification is in reference to co-precipitating pERK levels at 6 hours post-lesion. General ERK was used as a loading control. Experiments were repeated at least three times with similar results.

FIG. 17c is a set of Western blot photographs and an associated quantitative histogram depicting association of pERK with vimentin but not peripherin following axonal injury. Pull-down assays were performed in axoplasm 0-6 hours post-lesion using GST-pERK. GST-pERK (0.5 microgram) was incubated with 500 micrograms of axoplasm protein obtained at the indicated post-lesion times. The interaction was blocked by addition of EGTA (100 millimolar). Experiments were repeated at least three times with similar results. Experiments were repeated at least three times with similar results.

FIG. 17d is a set of Western blot photographs depicting retrograde movement of both pERK and vimentin with dynein until arrival in the DRG at 20 hours post-lesion. Co-immunoprecipitation of vimentin (left) and pERK (right) with Dynein from 300 microgram aliquots of axoplasm obtained from the sciatic nerve segments indicated indicated by the schematic. Sampling was performed at 0-24 hours post-lesion. At the 24-hour time point only vimentin was still found in association with dynein in the DRG. Dynein was used as a loading control for all lanes in the time series (not shown). Experiments were repeated at least three times with similar results.

FIG. 17e is a set of Western blot photographs depicting pElk-1 activation in L4/L5 DRGs from the experiment shown in FIG. 17d. Fifty microgram aliquots of DRG lysates from the indicated post-lesion times (hr) were analyzed by Western blot for pELK-1. General ERK expression was used as the loading control for this analysis. Experiments were repeated at least three times with similar results.

FIG. 18a is a set of Western blot photographs and an associated quantitative histogram depicting that the vimentin-pERK interaction is calcium dependent. GST-pERK (0.5 microgram) or GST-importin-beta (1 microgram) was incubated with vimentin (0.1 microgram) at the indicated calcium concentrations for 2 hours at 37 degrees centigrade. GST alone was used as negative control. The positive control (PC) is 50 nanograms vimentin. Experiments were repeated at least three times, and quantified as % of maximum (average plus/minus standard deviation).

FIG. 18b is a set of Western blot photographs and an associated quantitative histogram depicting concentration-dependent phosphatase protection of pERK by vimentin. His-tagged importin-beta (1 microgram) was incubated with pERK (0.5 microgram) and with the indicated amounts (micrograms) of vimentin or neurofilament for 2 hours at 37 degrees centigrade. Alkaline phosphatase (AP, 2 units) or axoplasm from injured nerve (AX, 100 micrograms) was added for an additional 30 minutes before performing pull-down on Nickel-NTA followed by Western blotting for pERK Vimentin protected pERK from dephosphorylation in a concentration dependent manner. Experiments were repeated at least three times, and quantified as % of maximum (average plus/minus standard deviation).

FIG. 18c is a set of Western blot photographs depicting that phosphatase protection of pERK by vimentin is calcium dependent. The experiment was carried out as described in FIG. 18b at a single concentration (2 micrograms) of vimentin or neurofilament and at the indicated concentrations of calcium. Note that calcium dependence of the phosphatases protection closely parallels the calcium dependence of vimentin-pERK binding shown in FIG. 18a. Experiments were repeated at least three times, and quantified as % of maximum (average plus/minus standard deviation).

FIG. 19a is a set of Western blot photographs depicting that pERK does not co-precipitate with dynein in sciatic nerve axoplasm from vimentin^{-/-} mice. Aliquots of 300 micrograms of axoplasm from control and 6 hours post-lesion nerve were subjected to dynein immunoprecipitation followed by Western blots for the indicated components. Note that although ERK phosphorylation is comparable in wild-type and vimentin^{-/-} mice, there is no interaction of pERK with the dynein complex in vimentin^{-/-} mice. This experiment was repeated three times with similar results.

FIG. 19b is an immunofluorescence photomicrograph depicting retrograde accumulation of pERK at a ligation site in injured sciatic nerve. Sciatic nerves of wild-type and vimentin^{-/-} mice underwent crush lesion and ligation between the lesion site and the ganglia. After 24 hours the nerves were removed and sectioned longitudinally over the ligation area. Sections were stained for NFH (green) and pERK (red). Note that pERK accumulates at the ligation site in wild-type nerve, but not in the vimentin-nulls. Magnification ×20. This experiment was repeated twice with similar results.

FIG. 19c is a set of Western blot photographs and an associated quantitative histogram depicting concomitant upregulation of both pERK and pElk-1 in ganglia of lesioned neurons from wild-type but not vimentin^{-/-} mice. DRG processes were lesioned approximately 1 mm from the ganglia. After incubation for the indicated times (minutes), ganglia were lysed and subjected to Western blot analyses as shown. The quantification is percent of maximum, average plus/minus standard deviation of three experiments.

FIG. 20a is a set of fluorescence photomicrographs and an associated quantitative histogram depicting reduced outgrowth of vimentin-null compared to wild-type triturated DRG neurons after 48 hours in culture. Green indicates immunostaining for NFH and red indicates vimentin (wild-type) or beta-Gal (vimentin^{-/-}). Magnification 20×, Each row includes two panels with predominantly vimentin or beta-Gal positive cells and two panels with cells predominantly negative for these markers. The third (lower) row shows vimentin^{-/-} neurons triturated with calpain-cleaved vimentin (6.7 micrograms per ganglion) before plating. A quantification of three such experiments (in each experiment at least 250 cells were measured) shown on the right reveals significant differences in neurite outgrowth between the vimentin-nulls wild-type in the total population (p<0.05). These differences are even more pronounced when comparing vimentin-positive to beta-Gal positive neurons (p<0.01), while there was no different between wild-type vimentin-negative and vimentin-null beta-Gal negative cells. Trituration of vimentin to vimentin-null neurons before plating enhanced the outgrowth of beta-Gal positive cells, and had no effect on cells that did not express the beta-Gal marker. Vimentin uptake after trituration was verified by immunostaining, and only those cultures in which over 50% of the cells contained vimentin 3 hours after trituration were scored.

FIG. 20b is a set of fluorescence photomicrographs and an associated quantitative histogram depicting effects of sciatic nerve conditional lesion in vimentin^{-/-} mice. Sciatic nerves were crushed as described and L4/L5 DRG neurons were cultured three days after the conditioning lesion. Neurons were fixed after 18 hours in culture, stained and neurite outgrowth was measured. Wild-type neurons and vimentin-null neurons negative for beta-Gal revealed accelerated neurite outgrowth after a conditioning crush (>100 neurons measured from two independent experiments, p<0.01), whereas no change was observed in vimentin-null beta-Gal-positive cells (250 neurons measured from two independent experiments).

FIG. 21 is a schematic diagram depicting a model for calcium regulated phosphatase protected retrograde transport of phosphorylated MAP kinases. Local synthesis of vimentin at the lesion site in the axon (left), concomitantly with phosphorylation (yellow) of ERK, allows linkage of pERK to the importins retrograde complex via a direct interaction of vimentin with importin-beta, in a complex that protects pERK from phosphatases due to steric hindrance. Phosphorylated ERK will remain bound to vimentin as long as calcium levels in the microenvironment are high. Upon arrival in the cell body where the stronger calcium buffering capacity restores basal calcium levels earlier than in the axon, the pERK will dissociate from vimentin and will then be available to substrates and downstream targets in the cell body or the nucleus.

FIG. 22 is a prior art schematic diagram (after Bogerd et al., 1999. J Biol Chem. 274:9771-7) depicting the structure of transportin (top structure), the consensus amino acid sequence (consensus; SEQ ID NO: 5) and the wild-type (wild-type; residues 266-277 of hnRNP A1; SEQ ID NO: 6) amino acid sequence of the M9 NLS found in transportin cargos. J, hydrophilic amino acid; Z, hydrophobic amino acid; X, any residue.

FIG. 23a is a Western blot photograph depicting that transportin is expressed in sciatic nerve axoplasm from adult rat, at similar levels before and after injury.

FIGS. 23b-c are fluorescence and phase-contrast photomicrographs, respectively, depicting transportin expression in corresponding DRG neurons in culture.

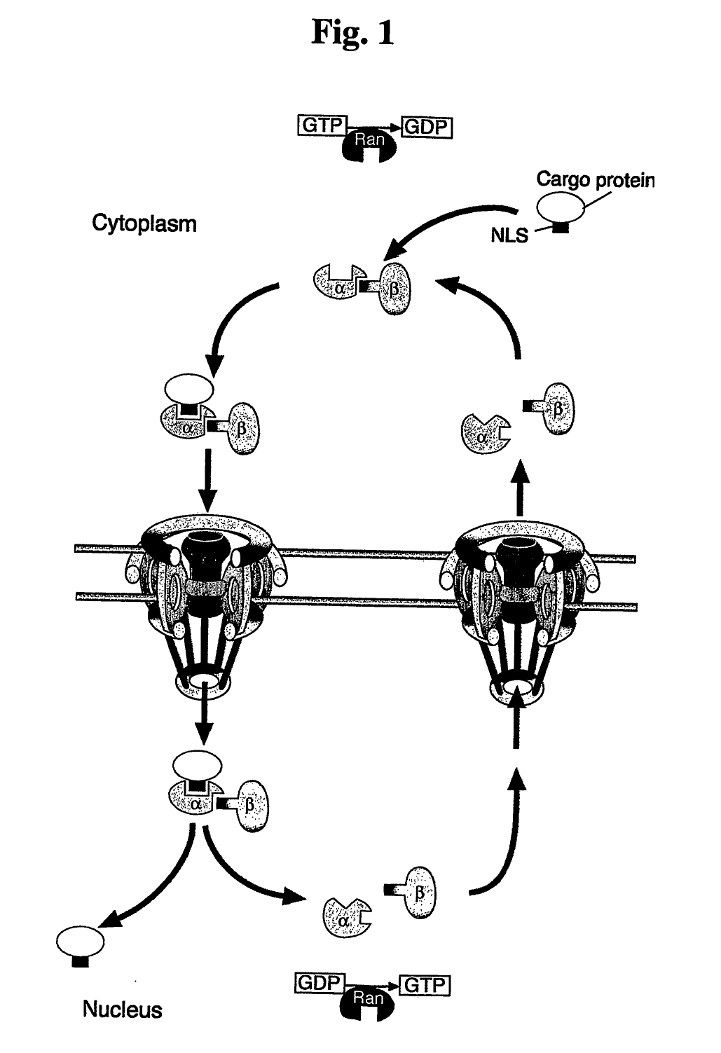

### DESCRIPTION OF THE PREFERRED EMBODIMENTS

The present invention is of: (i) a method of modulating growth of an axon; (ii) a method of inducing retrograde transport of an exogenous compound in an axon; (iii) a composition-of-matter comprising a compound which is associated with a molecule participating in importin mediated retrograde transport in an axon, and which is capable of regulating in a cell a physiological process such as growth, retrograde transport, survival, and/or differentiation; (iv) a polynucleotide encoding a chimeric polypeptide comprising at least a portion of a polypeptide which participates in importin mediated retrograde transport in an axon, and which is fused to an amino acid sequence capable of regulating a physiological process such as growth, retrograde transport, survival and/or differentiation in the axon; (v) a nucleic acid construct including such a polynucleotide; and (vi) a host cell transformed with such a construct. Specifically, the present invention can be used for modulating growth of an axon, and for inducing retrograde transport of a compound such as a therapeutic/diagnostic compound in an axon. As such, the present invention can be employed for optimally treating/diagnosing nervous system associated diseases.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

The principles and operation of the present invention may be better understood with reference to the drawings and accompanying descriptions.

Nervous system associated diseases include numerous highly debilitating and/or lethal diseases having a pathogenesis associated with a disregulated retrograde transport dependent physiological process, such as growth, survival and/or differentiation (for example, refer to: Heerssen H M. and Segal R A., 2002. Trends Neurosci. 25:160-5; Friedman W J. and Greene L A., 1999. Exp Cell Res. 253:131-42; Thoenen H., 1995. Science 270:593-8; Korsching S., 1993. J Neurosci. 13:2739-48), for which no satisfactory treatment and/or diagnostic method is available. An optimal strategy for treating/diagnosing such a disease would be to exploit/regulate axonal retrograde transport mechanisms in such a way as to: (i) therapeutically modulate in affected neurons such physiological processes; and/or (ii) deliver therapeutic/diagnostic compounds to neurons or neuron-associated cells via axonal retrograde transport.

Various methods of exploiting/regulating axonal retrograde transport mechanisms for modulating physiological processes such as growth, retrograde transport, survival and/or differentiation in neurons, or for inducing retrograde transport of exogenous compounds in neurons have been described by the prior art.

One approach has attempted utilizing administration of total protein from axoplasm of damaged axons for eliciting retrograde transport-dependent growth and survival responses in injured Aplysia neurons. Another approach involves attaching exogenous compounds to a nuclear localization signal (NLS) in attempts to induce retrograde transport of such proteins in axons. A further approach involves regulating in neurons signaling via the MAPK or Trk pathway in attempts to induce neuronal regeneration-enhancing retrograde signals. Yet a further approach involves using viral vectors in order to attempt to achieve retrograde delivery of vector-encoded exogenous protein.

However, as described above all of the aforementioned approaches suffer from significant disadvantages, including: (i) having no demonstrated applicability for use in mammalian neurons; (ii) being impractical or cumbersome to perform; (iii) having no demonstrated capacity for specifically inducing retrograde transport of exogenous compounds; (iv) being hazardous; and/or (v) having no demonstrated capacity for regulating neuronal growth.

Thus, the prior art has failed to provide a satisfactory method of regulating/exploiting retrograde transport for modulating physiological processes, such as growth, retrograde transport, survival and/or differentiation in neurons, or for inducing retrograde transport of exogenous compounds in neurons.

While reducing the present invention to practice it was uncovered for the first time that: (i) importin-alpha proteins are constitutively present throughout neuronal axons, at significant distances from the cell body; (ii) importin-beta protein is synthesized de-novo throughout neuronal axons, at significant distances from the cell body in injured axons, but is absent in non-injured, axons; (iii) formation of NLS:importin-alpha4 complex significantly increases in axons following injury thereof; (iv) soluble intermediate filament protein monomers, such as vimentin and peripherin, specifically accumulate in injured, but not in non-injured, axons and specifically complex in injured axons with activated ERK1 and ERK2 in a calcium dependent manner; (v) importin-beta directly binds to such intermediate filament protein monomer:ERK1/2 complex, NLS:importin-alpha4 complex, and the retrograde motor protein dynein to form a retrogradely transported complex comprising such components in injured axons; (vi) interference with formation of such a retrogradely transported complex induced by the presence of a competitor NLS-containing peptide prevents regenerative growth of injured axons. While reducing the present invention to practice it was further uncovered for the first time that the importin family member transportin is expressed in neurons, that such expression is with a juxtamembrane distribution and that expression levels of this molecule in neurons are unaffected by axonal injury.

While conceiving the present invention it was hypothesized that up-regulating or down-regulating importin mediated retrograde transport in an axon could be used for the first time for respectively up-regulating or down-regulating a physiological process in a neuron such as growth, retrograde transport, survival and/or differentiation. While conceiving the present invention, it was further hypothesized that retrograde transport of an exogenous compound, such as a regulatory/diagnostic compound, in an axon could be induced for the first time by increasing in the axon an activity and/or a level of a molecule participating in importin mediated regulated retrograde transport in conjunction with administering to the axon a compound capable of associating directly or indirectly with the molecule participating in importin mediated retrograde transport. While further conceiving the present invention, it was hypothesized modulating calcium levels could be used to control retrograde transport of activated ERKs or of conjugates thereof with essentially any desired cargo.

Hence, in sharp contrast to prior art techniques, the method of the present invention enables optimal up-regulation and down-regulation of a retrograde transport dependent physiological process in a neuron, such as growth, retrograde transport, survival and/or differentiation. In particular, the method according to the present invention enables optimal modulation of the growth of an axon, such as an injured axon. In sharp contrast to prior art techniques, the method of the present invention further enables optimal delivery of exogenous compounds, in particular therapeutic/diagnostic compounds, to neurons or neuron-associated cells via retrograde transport.

Thus, the method of the present invention can be used for optimally treating nervous system associated diseases whose pathogenesis is associated with a disregulated physiological process such as growth, retrograde transport, survival and/or differentiation. The method of the present invention can further be used for optimally treating/diagnosing nervous system associated diseases via retrograde transport delivery of therapeutic/diagnostic compounds. In particular, the method of the present invention can be used for optimally treating central nervous system nerve injury.

Thus, according to one aspect of the present invention there is provided a method of modulating growth of an axon. The method is effected by regulating importin mediated retrograde transport in the axon.

By virtue of enabling modulation of growth of an axon, the method according to this aspect of the present invention can be used for treating a disease whose pathogenesis is associated with insufficient or excessive growth of an axon. By virtue of enabling modulation of retrograde transport in an axon, the method according to this aspect of the present invention can further be used for facilitating inducing of retrograde transport of an exogenous compound in the axon, in particular a therapeutic/diagnostic compound, as described hereinbelow.

Regulating importin mediated retrograde transport in the axon may be effected in various ways, depending on the application and purpose. According to the teachings of the present invention, regulating importin mediated retrograde transport in the axon is preferably effected by altering (increasing/decreasing) in the axon an activity and/or a level of a molecule participating in importin mediated retrograde transport (referred to hereinunder as “retrograde transport molecule”).

Depending on the application and purpose, the retrograde transport molecule may be any of various retrograde transport molecules.

According to the teachings of the present invention, the retrograde transport molecule is preferably an importin, an intermediate filament protein, an ERK, or a molecule including a nuclear localization signal (NLS). Preferably, the retrograde transport molecule is of human origin.

Preferably, the importin is importin-alpha, or importin-beta.

Preferably, the importin-beta is importin-beta1.

Preferably, the importin-alpha is importin-alpha4.

Alternately, the importin may be a transportin, preferably transportin-1, (also termed MIP, described in Fridell, R A. et al., 1997. J. Cell Sci. 110, 1325-1331).

Preferably, the intermediate filament protein is a type III intermediate filament protein, more preferably peripherin, and most preferably vimentin.

Preferably, the ERK is ERK1 or ERK2.

Depending on the application and purpose, the ERK may be phosphorylated, preferably at two phosphorylation sites. As is illustrated in Example 1 of the Examples section which follows, the present inventors have clearly demonstrated for the first time that (i) importin-alpha proteins are found throughout neuronal axons, at significant distances from the cell body; (ii) importin-beta protein is synthesized de-novo throughout neuronal axons, at significant distances from the cell body in injured axons, but is not present in non-injured, axons; (iii) formation of NLS:importin-alpha4 complex significantly increases in axons following injury thereof; (iv) soluble intermediate filament proteins, such as vimentin and peripherin, specifically accumulate in injured, as opposed to non-injured, axons and specifically complex therein with activated (phosphorylated) ERK1 and ERK2; (v) importin-beta directly binds to such intermediate filament protein monomer:ERK1/2 complex, such NLS:importin-alpha4 complexes, and the retrograde motor protein dynein to form a retrogradely transported complex comprising such components, as demonstrated in injured axons; and (vi) formation of such a retrogradely transported complex is positively correlated with axonal growth, in particular regenerative axonal growth following axonal injury. As is illustrated in Example 2 of the Examples section which follows, the present inventors have further demonstrated that vimentin binding to phosphorylated ERKs enables calcium (Ca2+)-dependent dephosphorylation-protected transport of activated ERKs in the retrograde injury-signaling complex of axons. As is illustrated in Example 3 of the Examples section which follows, the present inventors have still further demonstrated that the importin family protein transportin is expressed at the juxtamembrane in neurons, and that levels of transportin in neurons is not substantially affected by neuronal injury.

Thus, the present results demonstrate that lesion-induced up-regulation of axonal importin-beta protein synthesis drives the formation of a retrogradely transported injury-signaling complex in damaged nerve. Such findings can therefore be directly exploited in numerous ways. In particular, regulation of levels of the aforementioned retrograde transport molecules, in particular those of importin-beta protein can readily be employed for up- or down-regulating retrograde transport, and thereby for regulating processes such as neuronal regeneration. Also, compounds such as diagnostic or therapeutic compounds, nucleic acids, viruses, etc., can be attached as cargo to such newly revealed retrograde transport complex constituents so as to be retrogradely delivered to neuronal cell bodies with the retrogradely transported complex. Such an approach is particularly advantageous for delivering cargo to inaccessible locations in the central nervous system, such as the brain. The aforementioned retrograde transport up-regulatory method and exogenous compound delivery method enabled by the presently described results can furthermore be used in combination to achieve optimal retrograde cargo delivery. Since association of activated ERKs with the retrogradely transported injury-signaling complex is calcium dependent, the present invention enables modulation of delivery of activated ERKs or of activated ERK:cargo conjugates, by regulating calcium levels in the axon. Such approaches can be used, for instance, for optimally delivering chemotherapeutic agents via axonal retrograde transport for targeting malignancies with known neurotropisms, especially during metastatic stages of such malignancies. Hence, the presently described method can be used for optimally diagnosing and treating numerous nervous system diseases.

As described in the Field and Background of the Invention section above, the prior art clearly teaches that the nuclear import function of importins indicates that such proteins should be found in a perinuclear distribution (Smith H M. and Raikhel N V., 1998. Plant Cell 10:1791-9; Lam M H. et al., 2002. Mol Endocrinol. 16:390-401; Mavlyutov T. A. et al., 2002. Traffic 3:630-40). Hence, the prior art teaches away from altering activities and/or levels molecules participating in importin mediated retrograde transport, such as the retrograde transport molecules of the present invention, in order to regulate retrograde dependent processes in neurons, such as growth. Similarly, the prior art also teaches away from employing molecules participating in importin mediated retrograde transport, such as the retrograde transport molecules of the present invention, in order to induce retrograde transport of an exogenous compound in an axon.

Several approaches can be utilized by the present invention to increase or decrease the level and/or activity of a retrograde transport molecule in a target neuron. The section below describes several examples of such approaches starting with methods which can be used to increase (upregulate), followed by methods which can be used to decrease (downregulate), the expression or activity of a retrograde transport molecule. Up-regulating the activity and/or level of the retrograde transport molecule according to the teachings of the present invention can be used for inducing growth of the axon, as described hereinbelow and in the Examples section below.

As described hereinabove, up-regulating in the axon the activity and/or level of the retrograde transport molecule can be effected in various ways, depending on the application and purpose

An agent capable of upregulating expression of a retrograde transport molecule may be an exogenous polynucleotide sequence designed and constructed to express at least a functional. portion of the retrograde transport molecule. Accordingly, the exogenous polynucleotide sequence may be a DNA or RNA sequence encoding such a retrograde transport molecule.

Retrograde transport molecules of the present invention have been cloned from human, rat and/or mouse sources [for example: human importin-beta1, Gorlich D. et al., Curr. Biol. 5(4), 383-392 (1995); human vimentin, Strausberg R L. et al., Proc. Natl. Acad. Sci. U.S.A. 99 (26), 16899-16903 (2002); human peripherin, Moncla A. et al., Genet. Res. 59 (2), 125-129 (1992); human ERK1, direct database submission/unpublished; human ERK2, Owaki H. et al., Biochem. Biophys. Res. Commun. 182 (3), 1416-1422 (1992); transportin, Pollard, V. W. et al., 1996. Cell 86, 985-994; Bonifaci, N. et al., 1997. Proc. Natl. Acad. Sci. U.S.A. 94, 5055-5060; Fridell, R. A. et al., 1997. J. Cell Sci. 110, 1325-1331]. Thus, coding sequence information for polynucleotides encoding retrograde transport molecules is available from several databases including the GenBank database available through http://www4.ncbi.nlm.nih.gov/.

To express an exogenous retrograde transport molecule in mammalian neuronal cells, a polynucleotide sequence encoding such a retrograde transport molecule (human importin-beta, GenBank Accession number NM_{—}002265; human vimentin, BC030573 or BC000163; human peripherin, GenBank Accession number NM_{—}006262; human ERK1, GenBank Accession number AY033608 or AY033607; human ERK2, GenBank Accession number M84489]; a plasmid (pGEM3-Trn) that permits efficient expression of full-length human transportin-1 in a coupled in vitro transcription/translation system has been described Fridell, R. A. et al., 1997. J. Cell Sci. 110, 1325-1331; a polynucleotide sequence encoding human transporting is described under GenBank Accession Number BC040340) is preferably ligated into a nucleic acid construct suitable for mammalian cell expression.

An agent capable of upregulating a retrograde transport molecule may also be any compound which is capable of up-regulating the transcription and/or translation of an endogenous DNA or mRNA encoding the retrograde transport molecule.

It will be appreciated that expressing or administering the retrograde transport molecule of the present invention or a functional portion thereof in an axon will facilitate formation of the retrogradely transported complex in the axon. Since, as shown in the Examples section below, retrograde transport in an injured axon is correlated with growth of the axon, expressing or administering at least the functional portion of the retrograde transport molecule in such an axon will enable up-regulation of growth thereof. Furthermore, since the retrograde transport molecule forms part of the retrogradely transported complex, and since the exogenous compound of the present invention is capable of associating with the retrograde transport molecule, expressing or administering the retrograde transport molecule or functional portion thereof in an axon will facilitate retrograde transport of such an exogenous compound when administered to the axon.

Administration of a retrograde transport molecule or functional portion thereof may be effected via any of various methods, for example via direct microinjection thereof into the axon as described and illustrated in the Examples section which follows.

It will be appreciated by the ordinarily skilled artisan that modulation of calcium levels according to the teachings of the present invention can be used to regulate transport of activated ERKs or of activated ERK:cargo conjugates to the nucleus in any of various suitable non-neuronal cell types.

In general, various methods may be used for administration. For example, for CNS administration across the blood brain barrier, disruption by surgery or injection, or drugs which transiently open adhesion contact between CNS vasculature endothelial cells, and compounds which facilitate translocation through such cells may be employed. Implants, such as collagen fibers, in osmotic pumps, and/or grafts comprising appropriately transformed cells, etc., may also be employed for the administration. Administration methods may employ coating, embedding or derivatizing fibers, such as collagen fibers, protein polymers, etc. with the administered compound, see also Otto et al. (1989) J Neuroscience Research 22, 83-91 and Otto and Unsicker (1990) J Neuroscience 10, 1912-1921. For administration in the context of spinal cord injuries, refer, for example to: Schulz M K, et al., Exp Neurol. 1998 February; 149(2): 390-397; Guest J D, et al., J Neurosci Res. 1997 Dec. 1; 50(5): 888-905; Schwab M E, et al., Spinal Cord. 1997 July; 35(7): 469-473; Tatagiba M, et al., Neurosurgery. 1997 March; 40(3): 541-546.

As described hereinabove, by virtue of enabling retrograde delivery of an exogenous compound such as a fluorophore-tagged antibody, the method of inducing retrograde transport of an exogenous compound of the present invention can be used for performing diagnostic immunohistochemistry of a neuron or neuron-associated cell. Ample guidance for practicing immunohistochemical techniques is provided in the literature of the art [for example, refer to: Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Harlow and Lane, “Antibodies: A Laboratory Manual”, Cold Spring Harbor Laboratory, New York, (1988)].

As described hereinabove, down-regulating in the axon the activity and/or level of the retrograde transport molecule may be effected in various ways.

One example, of an agent capable of down-regulating an activity of a retrograde transport molecule is an antibody or antibody fragment capable of specifically binding a portion of the retrograde transport molecule involved in retrograde transport. Preferably, the bound portion inhibits ligand-binding of the retrograde transport molecule. Preferably, the antibody specifically binds at least one epitope of a retrograde transport molecule. As used herein, the term “epitope” refers to any antigenic determinant on an antigen to which the paratope of an antibody binds.

Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)₂, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)₂, the fragment of the antibody that can be obtained by treating whole antibody with the enzyme pepsin without subsequent reduction; F(ab′)₂ is a dimer of two Fab′ fragments held together by two disulfide bonds; (4) Fv, defined as a genetically engineered fragment containing the variable region of the light chain and the variable region of the heavy chain expressed as two chains; and (5) Single chain antibody (“SCA”), a genetically engineered molecule containing the variable region of the light chain and the variable region of the heavy chain, linked by a suitable polypeptide linker as a genetically fused single chain molecule.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (1972)]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv or scFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFv's are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

Humanized forms of non-human (e.g., murine) antibodies are chimeric molecules of immunoglobulins, immunoglobulin chains or fragments thereof (such as Fv, Fab, Fab′, F(ab′)2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Another agent capable of down-regulating an activity and/or level of a retrograde transport molecule is a small interfering RNA (siRNA) molecule. An siRNA molecule functions to down-regulate an activity and/or level of a molecule via a process termed RNA interference. RNA interference is a two step process. the first step, which is termed as the initiation step, input double-stranded RNA (dsRNA) is digested into 21-23 nucleotide (nt)-long small interfering RNAs (siRNA), probably by the action of Dicer, a member of the RNase III family of dsRNA-specific ribonucleases, which processes (cleaves) dsRNA (introduced directly or via a transgene or a virus) in an ATP-dependent manner. Successive cleavage events degrade the RNA to 19-21 bp duplexes (siRNA), each with 2-nucleotide 3′ overhangs [Hutvagner and Zamore Curr. Opin. Genetics and Development 12:225-232 (2002); and Bernstein Nature 409:363-366 (2001)].

In the effector step, the siRNA duplexes bind to a nuclease complex to from the RNA-induced silencing complex (RISC). An ATP-dependent unwinding of the siRNA duplex is required for activation of the RISC. The active RISC then targets the homologous transcript by base pairing interactions and cleaves the mRNA into 12 nucleotide fragments from the 3′ terminus of the siRNA [Hutvagner and Zamore Curr. Opin. Genetics and Development 12:225-232 (2002); Hammond et al. (2001) Nat. Rev. Gen. 2:110-119 (2001); and Sharp Genes. Dev. 15:485-90 (2001)]. Although the mechanism of cleavage is still to be elucidated, research indicates that each RISC contains a single siRNA and an RNase [Hutvagner and Zamore Curr. Opin. Genetics and Development 12:225-232 (2002)].

Because of the remarkable potency of RNAi, an amplification step within the RNAi pathway has been suggested. Amplification could occur by copying of the input dsRNAs which would generate more siRNAs, or by replication of the siRNAs formed. Alternatively or additionally, amplification could be effected by multiple turnover events of the RISC [Hammond et al. Nat. Rev. Gen. 2:110-119 (2001), Sharp Genes. Dev. 15:485-90 (2001); Hutvagner and Zamore Curr. Opin. Genetics and Development 12:225-232 (2002)]. For more information on RNAi see the following reviews Tuschl ChemBiochem. 2:239-245 (2001); Cullen Nat. Immunol. 3:597-599 (2002); and Brantl Biochem. Biophys. Act. 1575:15-25 (2002).

Synthesis of RNAi molecules suitable for use with the present invention can be effected as follows. First, the retrograde transport molecule mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90 percent decrease in cellular GAPDH mRNA and completely abolished protein level (www.ambion.com/techlib/tn/91/912.html).

As used herein the term “about” refers to plus/minus 10 percent.

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55 percent. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

Another agent capable of downregulating an activity and/or level of a retrograde transport molecule is a DNAzyme molecule capable of specifically cleaving an mRNA transcript or DNA sequence encoding the retrograde transport molecule. DNAzymes are single-stranded polynucleotides which are capable of cleaving both single and double stranded target sequences (Breaker, R. R. and Joyce, G. Chemistry and Biology 1995; 2:655; Santoro, S. W. & Joyce, G. F. Proc. Natl, Acad. Sci. USA 1997; 943:4262). A general model (the “10-23” model) for the DNAzyme has been proposed. “10-23” DNAzymes have a catalytic domain of 15 deoxyribonucleotides, flanked by two substrate-recognition domains of seven to nine deoxyribonucleotides each. This type of DNAzyme can effectively cleave its substrate RNA at purine:pyrimidine junctions (Santoro, S. W. & Joyce, G. F. Proc. Natl, Acad. Sci. USA 199; for rev of DNAzymes see Khachigian, LM [Curr Opin Mol Ther 4:119-21 (2002)].

Examples of construction and amplification of synthetic, engineered DNAzymes recognizing single and double-stranded target cleavage sites have been disclosed in U.S. Pat. No. 6,326,174 to Joyce et al. DNAzymes of similar design directed against the human urokinase receptor were recently observed to inhibit urokinase receptor expression, and successfully inhibit colon cancer cell metastasis in-vivo (Itoh et al, 20002, Abstract 409, Ann Meeting Am Soc Gen Ther www.asgt.org). In another application, DNAzymes complementary to bcr-abl oncogenes were successful in inhibiting the oncogenes expression in leukemia cells, and lessening relapse rates in autologous bone marrow transplant in cases of CML and ALL.

Downregulating an activity and/or level of a retrograde transport molecule can also be effected by using an antisense polynucleotide capable of specifically hybridizing with an mRNA transcript encoding the retrograde transport molecule.

Design of antisense molecules which can be used to efficiently downregulate a retrograde transport molecule must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Luft J Mol Med 76: 75-6 (1998); Kronenwett et al. Blood 91: 852-62 (1998); Rajur et al. Bioconjug Chem 8: 935-40 (1997); Lavigne et al. Biochem Biophys Res Commun 237: 566-71 (1997) and Aoki et al. (1997) Biochem Biophys Res Commun 231: 540-5 (1997)].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)].

Such algorithms have been successfully used to implement an antisense approach in cells. For example, the algorithm developed by Walton et al. enabled scientists to successfully design antisense oligonucleotides for rabbit beta-globin (RBG) and mouse tumor necrosis factor-alpha (TNF alpha) transcripts. The same research group has more recently reported that the antisense activity of rationally selected oligonucleotides against three model target mRNAs (human lactate dehydrogenase A and B and rat gp130) in cell culture as evaluated by a kinetic PCR technique proved effective in almost all cases, including tests against three different targets in two cell types with phosphodiester and phosphorothioate oligonucleotide chemistries.

In addition, several approaches for designing and predicting efficiency of specific oligonucleotides using an in-vitro system were also published (Matveeva et al., Nature Biotechnology 16: 1374-1375 (1998)].

Several clinical trials have demonstrated safety, feasibility and activity of antisense oligonucleotides. For example, antisense oligonucleotides suitable for the treatment of cancer have been successfully used [Holmund et al., Curr Opin Mol Ther 1:372-85 (1999)], while treatment of hematological malignancies via antisense oligonucleotides targeting c-myb gene, p53 and Bcl-2 have been shown to be tolerated by patients in clinical trials [Gerwitz Curr Opin Mol Ther 1:297-306 (1999)].

More recently, antisense-mediated suppression of human heparanase gene expression has been reported to inhibit pleural dissemination of human cancer cells in a mouse model [Uno et al., Cancer Res 61:7855-60 (2001)].

Thus, the current consensus is that recent developments in the field of antisense technology which, as described above, have led to the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Another agent capable of downregulating an activity and/or level of a retrograde transport molecule is a ribozyme molecule capable of specifically cleaving an mRNA transcript encoding a retrograde transport molecule. Ribozymes are being increasingly used for the sequence-specific inhibition of gene expression by the cleavage of mRNAs encoding proteins of interest [Welch et al., Curr Opin Biotechnol. 9:486-96 (1998)]. The possibility of designing ribozymes to cleave any specific target RNA has rendered them valuable tools in both basic research and therapeutic. applications. In the therapeutics area, ribozymes have been exploited to target viral RNAs in infectious diseases, dominant oncogenes in cancers and specific somatic mutations in genetic disorders [Welch et al., Clin Diagn Virol. 10:163-71 (1998)]. Most notably, several ribozyme gene therapy protocols for HIV patients are already in Phase 1 trials. More recently, ribozymes have been used for transgenic animal research, gene target validation and pathway elucidation. Several ribozymes are in various stages of clinical trials. ANGIOZYME was the first chemically synthesized ribozyme to be studied in human clinical trials. ANGIOZYME specifically inhibits formation of the VEGF-r (Vascular Endothelial Growth Factor receptor), a key component in the angiogenesis pathway. Ribozyme Pharmaceuticals, Inc., as well as other firms have demonstrated the importance of anti-angiogenesis therapeutics in animal models. HEPTAZYME, a ribozyme designed to selectively destroy Hepatitis C Virus (HCV) RNA, was found effective in decreasing Hepatitis C viral RNA in cell culture assays (Ribozyme Pharmaceuticals, Incorporated—http://www.rpi.com).

Down-regulating in the axon the activity and/or level of the retrograde transport molecule may be advantageously effected by administering to the axon a molecule capable of inhibiting ligand-binding of the retrograde transport molecule. Preferably, the molecule capable of inhibiting ligand-binding of the retrograde transport molecule is a portion of the retrograde transport molecule or a portion of the ligand thereof capable of interfering with association of the retrograde transport molecule with its ligand. For example, as is described in Example 1 of the Examples section below, the use of an essentially isolated NLS can be used for preventing association of NLS containing polypeptides in the retrogradely transported complex and thereby for preventing retrograde transport dependent growth of an injured neuron. As is described in Example 2 of the Examples section which follows, downregulation of calcium levels may be employed to prevent association of activated ERK with the retrogradely transported complex.

For applications of the present invention involving altering an activity and/or level of a retrograde transport molecule of the present invention in an axon, the molecule including the NLS may be any of various molecules including an NLS endogenous to the axon in which such alteration is effected.

Preferably, the NLS includes the amino acid sequence set forth in SEQ ID NO: 2.

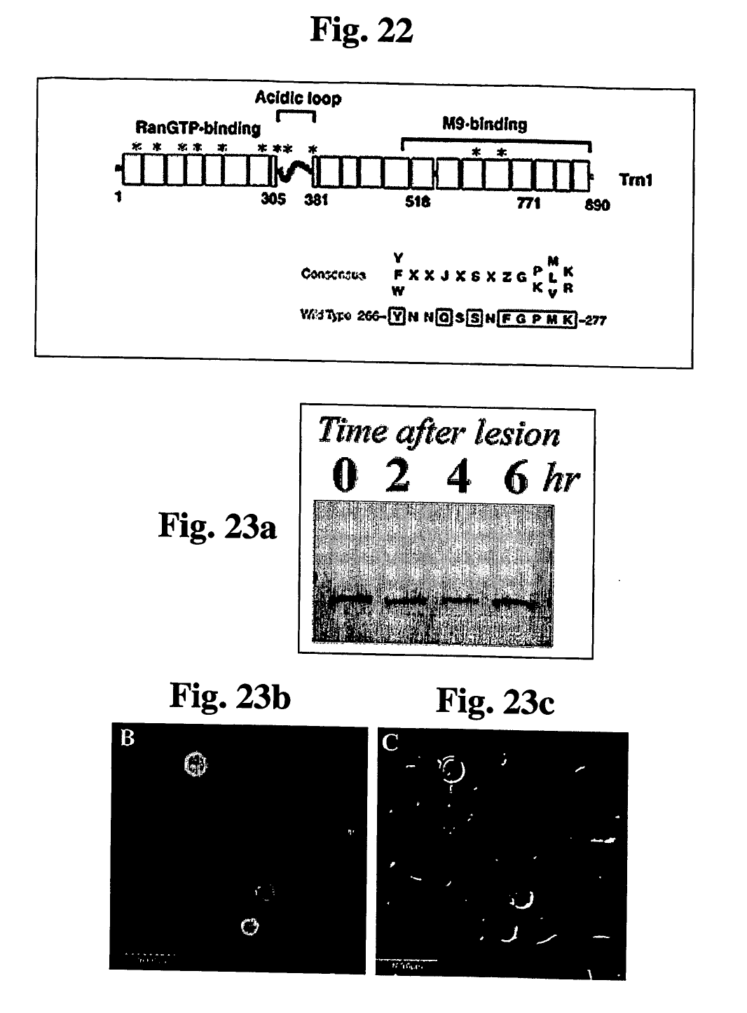

Alternately, the NLS may include any amino acid sequence defined by the M9 consensus sequence (SEQ ID NO: 5, described in Bogerd et al., 1999. J Biol Chem. 274:9771-7 and shown in FIG. 22 of the Examples section below), or more preferably the amino acid sequence of the wild-type M9 NLS (SEQ ID NO: 6, described in Bogerd et al., 1999. J Biol Chem. 274:9771-7 and shown in FIG. 22 of the Examples section below).

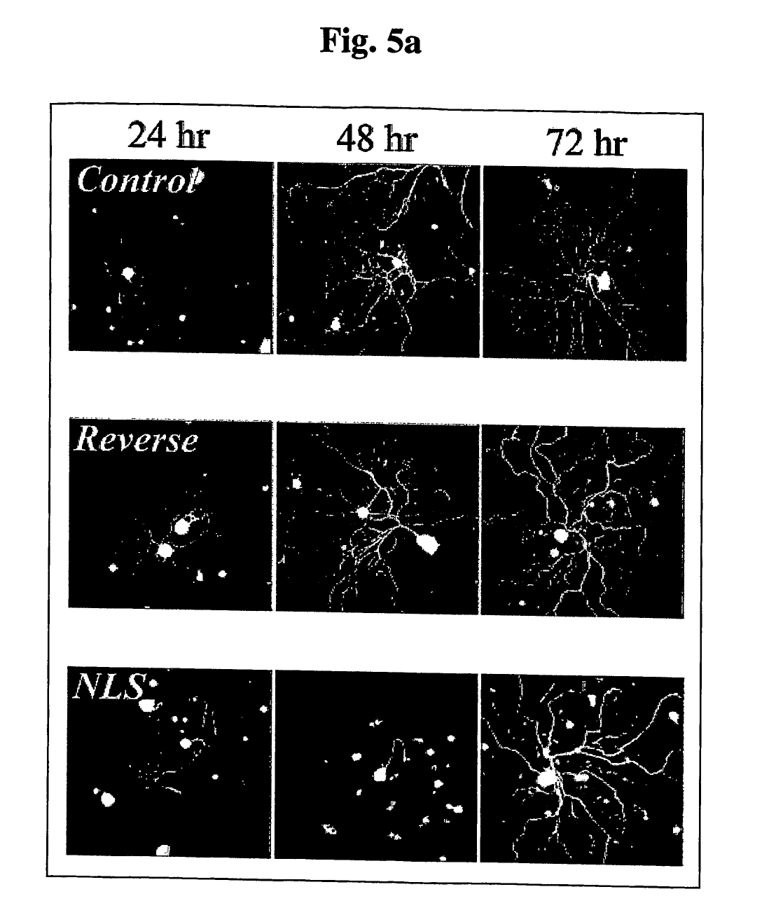

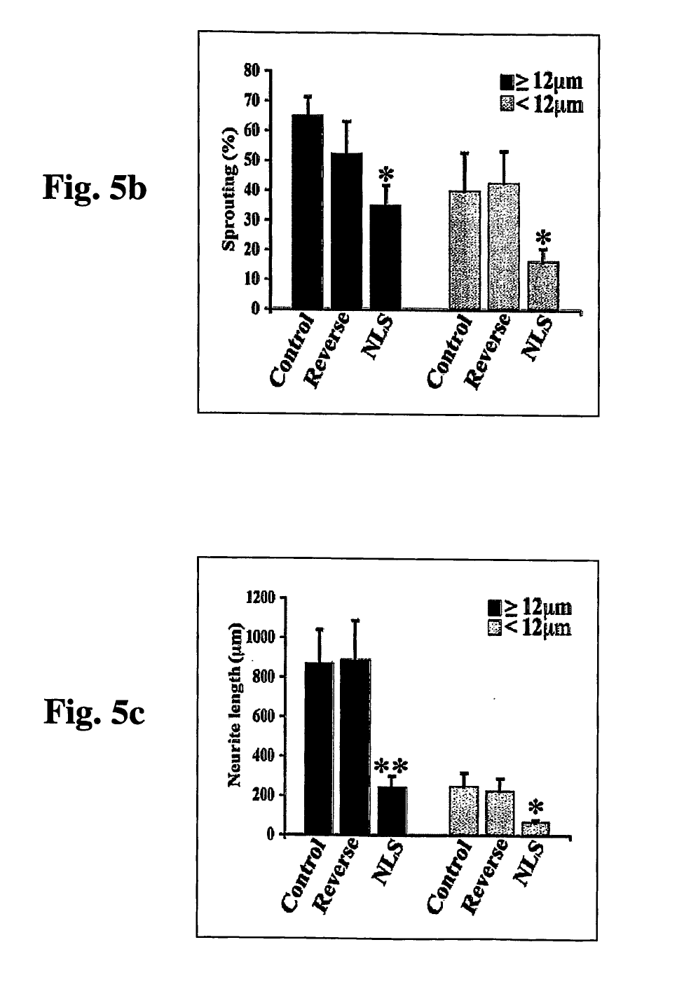

As is described and illustrated (FIGS. 5a-c) in the Examples section below, administering to an injured axon a molecule including an NLS which includes the amino acid sequence set forth in SEQ ID NO: 2 may be employed for modulating regenerative growth in such an axon.

A number of chimeric NLS derived peptides have already been described in the literature, including membrane-permeable (Lin, Y. Z. et al. 1995. J Biol Chem. 270:14255-8) or photoactivatable (Park, S. B., and R. F. Standaert. 2001. Bioorg Med Chem. 9:3215-23) variants. More recently the synthesis of simple organic mimetics of NLS was described (Park, S. B. et al., 2003. Org Lett. 5:2437-40), thus both peptide and nonpeptide targeting moieties are readily available. Administration of the M9 sequence to neurons may be performed as previously described (refer, for example, to Ma et al., Neuroscience. 2002; 112(1):1-5).

Techniques for administering proteins and oligonucleotides of the above described techniques for regulating the activity and/or level of the retrograde transport molecule to a cell or cellular structure are routinely practiced by the ordinarily skilled artisan.

A wide variety of methods may be used for administering to an axon such a protein or oligonucleotide. For example, for CNS administration across the blood brain barrier, disruption by surgery or injection, or drugs which transiently open adhesion contact between CNS vasculature endothelial cells, and compounds which facilitate translocation through such cells may be employed. Implants, such as collagen fibers, in osmotic pumps, and/or grafts comprising appropriately transformed cells, etc., may also be employed for the administration. Administration methods may employ coating, embedding or derivatizing fibers, such as collagen fibers, protein polymers, etc. with the protein or oligonucleotide, see also Otto et al. (1989) J Neuroscience Research 22, 83-91 and Otto and Unsicker (1990) J Neuroscience 10, 1912-1921. For administration in the context of spinal cord injuries, refer, for example to: Schulz M K, et al., Exp Neurol. 1998 February; 149(2): 390-397; Guest J D, et al., J Neurosci Res. 1997 Dec. 1; 50(5): 888-905; Schwab M E, et al., Spinal Cord. 1997 July; 35(7): 469-473; Tatagiba M, et al., Neurosurgery. 1997 March; 40(3): 541-546.

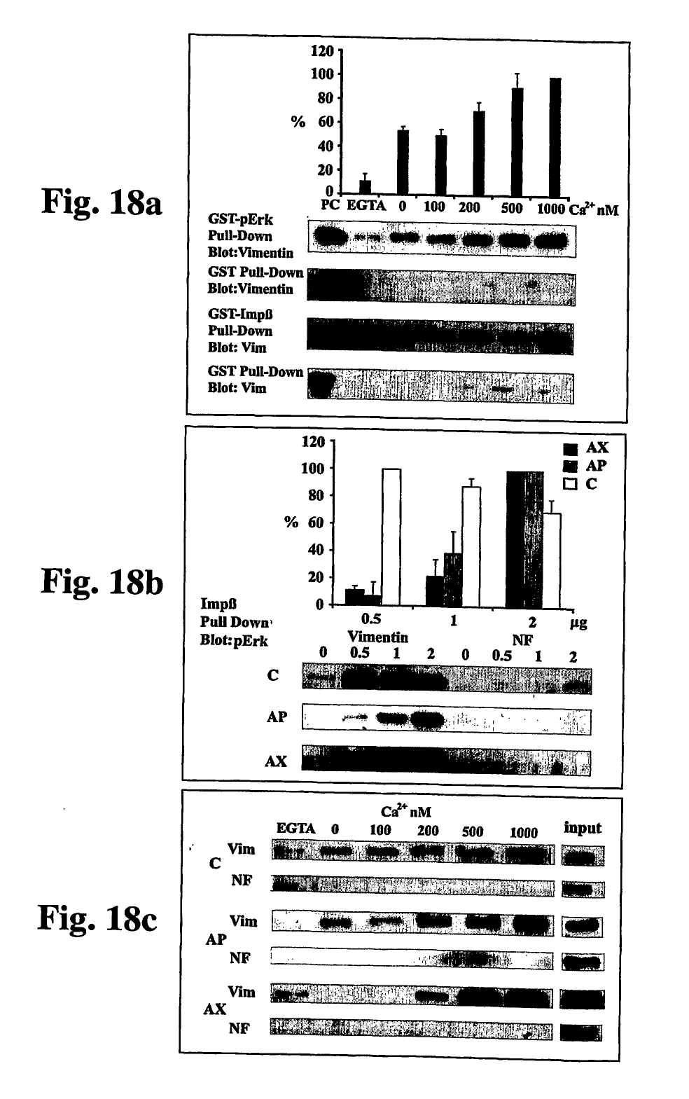

Administration of calcium to an axon may effected via any of various standard art methods, for example via direct microinjection. Alternately, calcium levels in an axon may be regulated via pharmacological modulation of calcium channels according to standard art methods (reviewed, for example in, Spedding M, Lepagnol J. Biochem Soc Trans. 1995 August; 23(3):633-6). As is shown in Example 3 (FIG. 18a) of the Examples section, below, calcium concentrations up to 1 millimolar can be used to induce association of activated ERK2 with the retrogradely transported injury complex via direct interaction with vimentin. Calcium levels may be down-regulated in an axon, for example, using a calcium chelator such as 1,2-bis(2-aminophenoxy) ethane-N,N,N′,N′-tetraacetic acid (BAPTA; refer, for example, to Balaban P M et al., Eur J Neurosci. 2004 January; 19(2):227-33).

It will be appreciated that a nucleic acid construct of the present invention can be administered to an individual employing any suitable mode of administration, described hereinabove (i.e., in-vivo gene therapy). Alternatively, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex-vivo gene therapy).

To enable cellular expression of the retrograde transport molecule, the nucleic acid construct of the present invention further includes at least one cis acting regulatory element, as described hereinabove. In a preferred embodiment of the present invention, the promoter utilized by the nucleic acid construct according to this aspect of the present invention is active in the specific cell population transformed. A suitable neuron specific promoter can be, for example, a gonadotropin-releasing hormone (GnRH; see Spergel D J. et al., 2001. Prog Neurobiol. 63:673-86), synapsin-1 bugler et al., Gene Ther. 2003 February; 10(4):337-47), or neuron-specific enolase (Andersen et al., Cell Mol Neurobiol. 1993 October; 13(5):503-15) promoter which is capable of directing neuron/neuronal subpopulation specific gene expression. The nucleic acid construct of the present invention can further include an enhancer, which can be adjacent to or distant relative to the promoter sequence and can function in up-regulating the transcription therefrom.

Currently preferred in-vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses. A viral construct such as a retroviral construct includes at least one transcriptional promoter/enhancer or locus-defining element(s), or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. The construct may include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Guidance for practicing such techniques is provided hereinbelow.

Any of the above described approaches for increasing the activity or expression of a retrograde transport molecule of the present invention can be utilized in increasing retrograde transport, and thereby neuronal regeneration, growth and/or differentiation.

For example, breakdown or impairment of axonal transport mechanisms, such as retrograde transport mechanisms, has been implicated in the initiation or progression of a number of neurodegenerative diseases (Gunawardena, S., and L. S. Goldstein. 2004. J Neurobiol. 58:258-71; Salehi, A. et al., 2003. Trends Neurosci. 26:73-80). As such the present invention can be used to induce therapeutic upregulation of such axonal transport mechanisms in the context of such neurodegenerative diseases.

Also, it has been shown that corticospinal neurons up-regulate a number of growth-associated genes following intracortical axotomy, but do not respond to spinal injury (Mason, M. R. et al., 2003. Eur J Neurosci. 18:789-802). The present inventors predict that these central neurons are specifically impaired in long distance retrograde injury signaling, and hence that upregulation of retrograde transport according to the teachings of the present invention provides a new option for therapeutic intervention in central nervous system lesions.

Any of the above described approaches for decreasing the activity or expression of a retrograde transport molecule of the present invention can be utilized for inhibiting growth of an axon.

Down-regulating the activity and/or level of the retrograde transport molecule according to the teachings of the present invention can be used for inhibiting growth of the axon, as described hereinbelow and in the Examples section which follows. As is described and illustrated (FIGS. 5a-c) in the Examples section below, regulating importin mediated retrograde transport in an axon according to the teachings of the present invention, for example by altering the activity and/or level of importin-beta1, can be used for modulating regenerative growth of an injured axon.

Downregulating retrograde transport may be relevant for various clinically Important syndromes in the nervous system for which there is evidence of involvement of retrograde mechanisms. For example neuropathic pain is associated with increased hyperexcitability in damaged sensory nerves which typically appears hours to days after the initial injury, and which can be blocked in model systems by microtubule disruptors, indicating that a retrograde signal underlies critical aspects of the etiology (Ji, R. R. et al., 2003. Trends Neurosci. 26:696-705; Sung, Y. J., and R. T. Ambron. 2004. Neurol Res. 26:195-203). Also, viral invasion of the nervous system is often dependent both on dynein-mediated transport along axon microtubules and on nuclear import (Dohner, K et al., 2002. Mol Biol Cell. 13:2795-809). Therefore certain viruses influence importin levels in axons as a combined mechanism to facilitate both retrograde transport and nuclear import. As such, regulation of such importin-mediated retrograde transport according to the teachings of the present invention may be used to treat neuropathic pain or diseases associated with such viruses.

The composition-of-matter of the present invention can be used per se or it can be formulated as the active ingredient of a pharmaceutical composition comprising suitable carriers and/or diluents, and an effective concentration of the compound of the present invention so as to be suitable for treating/diagnosing a nervous system associated disease when suitably administered to a subject in need of such treatment/diagnosis.

As used herein a “pharmaceutical composition” refers to a preparation of one or more of the active ingredients described herein with other chemical components such as physiologically suitable carriers and excipients. The purpose of a pharmaceutical composition is to facilitate administration of active ingredients to an organism.

Herein the term “active ingredients” refers to the regulator compound of the present invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered active ingredients. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intravenous, inrtaperitoneal, intranasal, or intraocular injections.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (regulator compound) effective to prevent, alleviate or ameliorate symptoms of a disorder (e.g., ischemia) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in-vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in-vitro, in cell cultures or experimental animals. The data obtained from these in-vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide plasma or brain levels of the active ingredients are sufficient to achieve a desired biological/diagnostic/therapeutic effect (minimal effective concentration, MEC). The MEC will vary for each preparation, but can be estimated from in-vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Since the present inventors have clearly identified for the first time novel molecules forming a retrogradely transported complex, the present invention also contemplates the use of conjugates of a retrograde transport molecule of the present invention with a cargo, such as an exogenous compound (retrograde transport molecule:cargo complex) for the purpose directing the retrograde transport of such exogenous compounds, for example to achieve retrograde delivery of such an exogenous compound to the neuron body and nucleus following administration of the conjugate to the axon.

Thus, according to another aspect of the present invention there is provided a method of inducing retrograde transport of an exogenous compound in an axon.

The method is effected in a first step by increasing in the axon an activity and/or a level of a molecule participating in importin mediated retrograde transport. In a second step, the method is effected by administering the exogenous compound to the axon. According to the teachings of the present invention, the exogenous compound is capable of directly or indirectly associating with the molecule participating in importin mediated retrograde transport.

As used herein, the phrase “exogenous compound” includes any molecule, substance, or particle exogenous with respect to the axon.

As described hereinabove, the method according to this aspect of the present invention can be used for optimally delivering a compound, such as a diagnostic/therapeutic compound, to a neuron or neuron-associated cell via retrograde transport. Hence, by optimally enabling such delivery, the method can be used for optimally diagnosing/treating a nervous system associated disease, in particular a disease in which pathologically affected neurons and/or neuron-associated cells are inadequately accessible to surgical and/or pharmacological treatment.

By virtue of enabling retrograde delivery of an exogenous compound such as a fluorophore-tagged antibody, the method can be used for performing diagnostic immunohistochemistry of a neuron or neuron-associated cell. Methods of employing immunohistochemistry for diagnosis or staging of a disease, are routinely practiced by one of ordinary skill in the art. Guidance for practicing such immunohistochemical detection is provided hereinbelow and in the Examples section which follows.

Depending on the application and purpose, administering the exogenous compound to the axon may be effected prior to, concomitantly with, or following the step of up-regulating in the axon the activity and/or level of the retrograde transport molecule.

Where the molecule participating in importin mediated retrograde transport is phosphorylated ERK, the method may advantageously further comprise the step of modulating in the axon a level of calcium. As is shown in Example 2 of the Examples section below, binding of phosphorylated ERK to the retrograde transport machinery via vimentin is calcium dependent. As mentioned above, since association of activated ERKs with the retrogradely transported injury-signaling complex is calcium dependent, it is possible to modulate delivery of activated ERKs or of activated ERK:cargo conjugates, by regulating calcium levels in the axon.

The method according to this aspect of the present invention may be effected by inducing retrograde transport of an exogenous compound of the present invention in any of various types of axons so as to induce retrograde transport of the exogenous compound in such axons. Preferably, the method according to this aspect of the present invention is effected by inducing retrograde transport of an exogenous compound of the present invention in an injured axon so as to induce retrograde transport of the exogenous compound therein.

As described hereinabove, practicing the method of the present invention of inducing retrograde transport of an exogenous compound in an axon may be effected using any of various retrograde transport molecules of the present invention. Preferably, the retrograde transport molecule employed for practicing the method is vimentin. As is described and illustrated (FIG. 10) of the Examples section below, administering exogenous vimentin to an injured axon in which an activity and a level of a retrograde transport molecule (e.g., importin-beta1) is up-regulated can be used for inducing retrograde transport of the administered vimentin in the axon. As described in the Examples section below, vimentin is capable of associating with importin-beta1 whose activity and level are up-regulated in an injured neuron.

Administration of a retrograde transport molecule:cargo complex of the present invention may be coupled with controlled up-regulation of importin-beta at the site of introduction to enable optimal retrograde delivery of cargo, such as a therapeutic or diagnostic agent, to a location, such as the CNS.

It will be appreciated that essentially any exogenous compound can be selected intrinsically capable of, or can modified so as to be capable of, associating to essentially any retrograde transport molecule of the present invention. Hence, the method according to this aspect of the present invention can be used for inducing retrograde transport of essentially any exogenous compound in an axon, including essentially any therapeutic/diagnostic compound. Therefore, the method according to this aspect of the present invention can be used for treating/diagnosing any of various nervous system associated diseases amenable to treatment/diagnosis via retrograde delivery of such a therapeutic/diagnostic compound.

Thus, according to yet another aspect of the present invention there is provided a composition-of-matter comprising a compound associated with a molecule participating in importin mediated retrograde transport in an axon, where the compound is capable of regulating a physiological process in a cell such as growth, retrograde transport, survival and/or differentiation.

A compound of the present invention capable of regulating a physiological process in a cell such as growth, retrograde transport, survival and/or differentiation is hereinafter referred to as “regulator compound”.

A composition-of-matter of the present invention comprising a regulator compound of the present invention can be utilized for various purposes involving regulation of a physiological process such as growth, retrograde transport, survival and/or differentiation in a cell. Preferably, such a composition-of-matter is used for treating a disease whose pathogenesis is associated with disregulated growth, retrograde transport, survival and/or differentiation in a neuron or neuron-associated cell.

Depending on the application and purpose, the composition-of-matter may advantageously further comprise calcium. As described above, association of activated ERKs with the retrogradely transported injury-signaling complex, via direct interaction with vimentin, is calcium dependent. As such, a composition-of-matter of the present invention comprising calcium may be employed to induce association of activated ERKs, or of activated ERK:cargo conjugates, with the retrogradely transported injury signaling complex.

Depending on the application and purpose, the composition-of-matter of the present invention may comprise any of various retrograde transport molecules of the present invention and any of various regulator compounds of the present invention. As described hereinabove, retrograde transport of a regulator compound of the present invention can be induced by association thereof with a retrograde transport molecule of the present invention. Compounds suitable for regulating in a cell a physiological process such as growth, retrograde transport, survival, and/or differentiation are well known to the ordinarily skilled artisan. An ordinarily skilled physician or pharmacologist would possess the necessary expertise for selection of a regulator compound whose retrograde transport in an axon could be used for treating a given disease.

According to this aspect of the present invention, the regulator compound may be selected intrinsically capable of associating with the retrograde transport molecule, and/or may be suitably modified so as to be rendered capable of associating to a desired degree of effectiveness with the retrograde transport molecule.

A polypeptidic retrograde transport molecule of the present invention forming part of the retrogradely transported complex uncovered while reducing the present invention to practice, and a regulator compound of the present invention also forming part of such a retrogradely transported complex will in specific cases, as described hereinbelow, and as described and illustrated in the Examples section which follows, be intrinsically capable of effectively associating with each other. It will be appreciated that in such cases the regulator compound may advantageously not require modification to render it capable of effectively associating with the retrograde transport molecule.

A regulator compound of the present invention may be modified so as to be rendered capable of associating to a desired degree of effectiveness with the retrograde transport molecule in various ways, depending on the physico-chemical characteristics of the regulator compound and of the retrograde transport molecule.

Methods of modifying essentially any given compound, such as a regulator compound of the present invention, so as to render it capable of specifically associating with essentially any molecule, such as a retrograde transport molecule of the present invention, are well within the purview of one of ordinary skill in the art. For guidance regarding covalent attachment of molecules, refer, for example to the extensive guidelines provided by The American Chemical Society (http://www.chemistry.org/portal/Chemistry). One of ordinary skill in the art, such as, for example, a chemist, will possess the required expertise for practicing chemical techniques suitable for attaching essentially any pair of molecules to each other.

In cases where the both the regulator compound and the retrograde transport molecule are polypeptidic but intrinsically incapable of associating with each other, association therebetween may be optimally effected via translational fusion, which, it will be appreciated advantageously results in a covalent, and hence optimally stable, association.

Covalently associating a polypeptidic regulator compound of the present invention and a polypeptidic retrograde transport molecule of the present invention via translational fusion may be conveniently effected using a polynucleotide encoding a chimeric polypeptide comprising at least a functional portion of the polypeptidic retrograde transport molecule fused to the polypeptidic regulator compound.

Methods of associating distinct polypeptides, such as a portion of a polypeptidic regulator compound and a polypeptidic retrograde transport molecule of the present invention via translational fusion are routinely practiced by the ordinarily skilled practitioner.

Ample guidance for practicing such methods is provided in the literature of the art (for example, refer to: Sambrook et al., infra and associated references in the introduction to the Examples section below).

Thus, according to still another aspect of the present invention there is provided a polynucleotide encoding a chimeric polypeptide comprising at least a portion of a molecule participating in importin mediated retrograde transport in an axon. According to this aspect of the present invention, the portion of the molecule participating in importin mediated retrograde transport in an axon is fused to an amino acid sequence capable of regulating in a cell a physiological process such as growth, retrograde transport, survival, and/or differentiation.

Depending on the application and purpose, the polynucleotide may comprise any of various retrograde transport molecules, and any of various amino acid sequences capable of regulating in a cell a physiological process such as growth, retrograde transport, survival, and/or differentiation. As described hereinabove, regulator molecules, including polypeptidic regulator molecules such as the amino acid sequence according to this aspect of the present invention, suitable for regulating in a cell a physiological process such as growth, retrograde transport, survival, and/or differentiation are well known to the ordinarily skilled artisan.

According to the present invention, the polynucleotide of the present invention can be a genomic polynucleotide, a complementary polynucleotide, or a composite polynucleotide.

As used herein, the phrase “complementary polynucleotide” refers to a polynucleotide having a nucleic acid sequence resulting from reverse transcription of messenger RNA using a reverse transcriptase or any other RNA dependent DNA polymerase. Such sequences can be subsequently amplified in-vivo or in-vitro using a DNA dependent DNA polymerase.

As used herein, the phrase “genomic polynucleotide” refers to a polynucleotide derived from a chromosome which thereby reflects a contiguous portion of the chromosome.

As used herein, the phrase “composite polynucleotide” refers to a polynucleotide which is at least partially complementary and at least partially genomic. A composite sequence can include some exonic sequences required to encode the polypeptide of the present invention, as well as some intronic sequences interposed between the exonic sequences. The intronic sequences can be of any source and typically include conserved splicing signal sequences. Such intronic sequences may further include cis acting expression regulatory elements.

Preferably, the polynucleotide of the present invention is a complementary DNA (cDNA).

The polynucleotide of the present invention can be used for genetically directing the production of the chimeric polypeptide of the present invention in a variety of host cell types.

Preferably, the polynucleotide of the present invention is capable of driving expression of the chimeric polypeptide of the present invention in a neuron.

Insertion and/or expression of the polynucleotide of the present invention within the host cell is preferably effected by cloning the polynucleotide within a suitable nucleic acid construct.

Methods of cloning a polynucleotide, such as the polynucleotide of the present invention, within a nucleic acid construct is routinely practiced by the ordinarily skilled artisan. Ample guidance for practicing such methods is provided in the literature of the art (for example, refer to: Sambrook et al., infra and associated references in the introduction to the Examples section below).

Thus, according to a further aspect of the present invention there is provided a nucleic acid construct including the polynucleotide of the present invention.

The nucleic acid construct of the present invention can be used for genetically transforming a host cell therewith.

The polynucleotide of the present invention can be introduced into cells by any one of a variety of known methods within the art. Such methods can be found generally described in Sambrook et al., [Molecular Cloning: A Laboratory Manual, Cold Springs Harbor Laboratory, New York (1989, 1992)]; Ausubel et al., [Current Protocols in Molecular Biology, John Wiley and Sons, Baltimore, Md. (1989)]; Chang et al., [Somatic Gene Therapy, CRC Press, Ann Arbor, Mich. (1995)]; Vega et al., [Gene Targeting, CRC Press, Ann Arbor Mich. (1995)]; Vectors [A Survey of Molecular Cloning Vectors and Their Uses, Butterworths, Boston Mass. (1988)] and Gilboa et al. [Biotechniques 4 (6): 504-512 (1986)] and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods for inducing homologous recombination.

An advantageous approach for introducing a polynucleotide of the present invention into cells is by using a viral vector. Viral vectors offer several advantages including higher efficiency of transformation, and targeting to, and propagation in, specific cell types. Viral vectors can also be modified with specific receptors or ligands to alter target specificity through specific cell receptors, such as neuronal cell receptors (for example, refer to Kaspar B K et al., 2002. Mol Ther. 5:50-6).

Retroviral vectors represent one class of vectors suitable for use with the present invention. Defective retroviruses are routinely used in transfer of genes into mammalian cells (for review see Miller, A. D., Blood 76: 271 (1990)]. A recombinant retrovirus including a polynucleotide encoding the chimeric polypeptide of the present invention can be constructed using well known molecular techniques. Portions of the retroviral genome can be removed to render the retrovirus replication defective and the replication defective retrovirus can then packaged into virions, which can be used to infect target cells through the use of a helper virus and while employing standard techniques. Protocols for producing recombinant retroviruses and for infecting cells in-vitro or in-vivo with such viruses can be found in, for example, Ausubel et al., [eds, Current Protocols in Molecular Biology, Greene Publishing Associates, (1989)]. Retroviruses have been used to introduce a variety of genes into many different cell types, including neuronal cells, epithelial cells endothelial cells, lymphocytes, myoblasts, hepatocytes and bone marrow cells.

Another suitable expression vector may be an adenovirus vector. The adenovirus is an extensively studied and routinely used gene transfer vector. Key advantages of an adenovirus vector include relatively high transduction efficiency of dividing and quiescent cells, natural tropism to a wide range of epithelial tissues and easy production of high titers [Russel, W. C. [J. Gen. Virol. 81: 57-63 (2000)]. The adenovirus DNA is transported to the nucleus, but does not integrate thereinto. Thus the risk of mutagenesis with adeno viral vectors is minimized, while short term expression is particularly suitable for treating cancer cells, such as multidrug resistant cancer cells. Adenoviral vectors used in experimental cancer treatments are described by Seth et al. [Adenoviral vectors for cancer gene therapy. In: P. Seth (ed.) Adenoviruses: Basic biology to Gene Therapy, Landes, Austin, Tex., (1999) pp. 103-120].

A suitable viral expression vector may also be a chimeric adenovirus/retrovirus vector which combines retroviral and adenoviral components. Such vectors may be more efficient than traditional expression vectors for transducing tumor cells [Pan. et al., Cancer Letters 184: 179-188 (2002)].

A specific example of a suitable viral vector for introducing and expressing the polynucleotide sequence of the present invention in an individual is the adenovirus-derived vector Ad-TK. This vector expresses a herpes virus thymidine kinase (TK) gene for either positive or negative selection and includes an expression cassette for desired recombinant sequences. This vector can be used to infect cells that have an adenovirus receptor which includes most cancers of epithelial origin (Sandmair et al., 2000. Hum Gene Ther. 11:2197-2205).

Features that limit expression to particular cell types can also be included. Such features include, for example, promoter and regulatory elements that are specific for the desired cell type. Secretion signals generally contain a short sequence (7-20 residues) of hydrophobic amino acids. Secretion signals suitable for use in this invention are widely available and are well known in the art, see, for example by von Heijne [J. Mol. Biol. 184:99-105 (1985)] and by Lej et al., [J. Bacteriol. 169: 4379 (1987)].

The recombinant vector can be administered in several ways. If viral vectors are used the procedure can take advantage of their target specificity and consequently, such vectors do not have to be administered locally. However, local administration can provide a quicker and more effective treatment. Administration of viral vectors can also be performed by, for example, intravenous or subcutaneous injection into the subject. Following injection, the viral vectors will circulate until they recognize host cells with appropriate target specificity for infection.

The nucleic acid construct of the present invention can be used for genetically expressing the chimeric polypeptide of the present invention in a host cell transformed therewith.

Thus, according to yet a further aspect of the present invention there is provided a host cell transformed with the nucleic acid construct of the present invention.

Techniques for genetically transforming a cell such as a neuron or neuron-associated cell with a nucleic acid construct are routinely practiced by the ordinarily skilled artisan. Ample guidance for practicing such methods is provided in the literature of the art (for example, refer to: Sambrook et al., infra and associated references in the introduction to the Examples section below).

Preferably, the host cell is a neuron.

The host cell may be genetically transformed with the nucleic acid construct of the present invention ex-vivo or in-vivo. In the ex-vivo approach, cells are removed from an individual and transformed with the polynucleotide of the present invention while being cultured.

A host cell transformed with the nucleic acid construct of the present invention ex-vivo may subsequently be conveniently propagated in culture so as to generate an expanded population thereof.

It will be appreciated that an expanded population of host cells, in particular neuronal host cells, transformed with a suitable nucleic acid construct of the present invention can be administered to a subject having a disease, in particular a nervous system associated disease, in such a way as to treat such a disease in the subject. An ordinarily skilled physician will possess the necessary expertise for therapeutic administration of a host cell transformed with a suitable nucleic acid construct of the present invention so as to treat a disease.

As described hereinabove, depending on the application and purpose, the various aspects of the present invention may be practiced by using, and/or by altering an activity and/or level of any of various retrograde transport molecules of the present invention.

Thus, by virtue of optimally enabling regulation of retrograde transport and by optimally enabling retrograde transport delivery of essentially any therapeutic/diagnostic compound in an axon, the present invention can be used for optimally treating/diagnosing nervous system associated diseases, such as neurodegenerative, malignant, infectious, stroke-associated, physical injury-induced, and developmental diseases. In particular, the present invention can be used for optimally modulating regeneration of injured nerves. It will be appreciated that by virtue of enabling regulation of importin mediated transport, which is a central element of retrograde transport in general in neurons, as described while reducing the present invention to practice, the present invention can be used for characterizing essentially any aspect of neuronal biology involving retrograde transport.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### Example 1

### Importin-Beta1 is a Central Regulator of Retrograde Transport

As described in the Field and Background of the Invention section above, nervous system diseases include numerous highly debilitating and/or lethal diseases, including major diseases, whose pathogenesis is associated with disregulated retrograde transport associated physiological processes in neurons for which no satisfactory treatment and/or diagnostic method is available. An optimal strategy for treating/diagnosing such diseases would be to exploit/regulate neuronal retrograde transport mechanisms to deliver therapeutic/diagnostic compounds to neuronal cell bodies, such as neuronal cell bodies difficult to access or localize. Regulation of retrograde transport mechanisms could also used for optimally regulating physiological processes of neurons generally dependent on retrograde transport, such as growth, survival, and/or differentiation. While various prior art approaches have attempted or suggested methods of exploiting/regulating neuronal retrograde transport mechanisms for achieving such objectives, none have proven to be satisfactory for the various reasons outlined in the Field and Background of the Invention section, above. While reducing the present invention to practice, the present inventors unexpectedly elucidated fundamental mechanisms of retrograde transport whose exploitation/regulation can be used for delivering exogenous compounds to neuronal cell bodies, and for regulating neuronal regeneration, thereby overcoming the limitations of the prior art, as described below (and as also described in Hanz, S. et al., 2003. Neuron. 40:1095-104).

Materials and Methods:

Animals and experimental system: Adult male Wistar rats were sacrificed by cervical dislocation. The sciatic nerves were dissected, cut into several pieces and incubated at 37 degrees centigrade and 5% carbon dioxide in HEPES-buffered DMEM (Biological Industries, Beit HaEmek, Israel). Axoplasm was then obtained by gentle extrusion from the nerve segments in nuclear transport buffer (NTB; 20 millimolar HEPES, pH 7.3, 110 millimolar potassium acetate, 5 millimolar magnesium acetate and 0.5 millimolar EGTA) or in phosphate buffered saline (PBS) containing protease inhibitors (Roche). Axoplasm from control nerves was harvested immediately following dissection.

Antibodies, Western blotting, and immunofluorescence microscopy: Proteins from axoplasm samples were resolved via 10% SDS-PAGE, blotted onto nitrocellulose membranes, and probed with anti importin-alpha1, -alpha2, -alpha4 and -alpha7 rabbit polyclonal antibodies (Kohler, M. et al., 1997. FEBS Lett. 417:104-108; Kohler, M. et al., 1999. Molecular and Cellular Biology 19:7782-7791). Importin-alpha4 was detected using polyclonal anti importin-alpha4 antibody (a kind gift from Dr. Karsten Weis, University of California at Berkeley); importin-beta1 (referred to hereinafter as “importin-beta”) was detected using the mouse anti importin-beta monoclonal antibody 3E9 (Affinity Bioreagents, Golden, Colo.); and a conserved epitope on the 74 kDa intermediate chain of cytoplasmic dynein was detected using the monoclonal antibody 74.1 (Chemicon, Temecula, Calif.). For secondary antibody detection, HRP-conjugated anti rabbit and anti mouse antibodies were used (Bio-Rad, Hercules, Calif.). Blots labeled with secondary antibodies were developed using ECL (Pierce). For immunofluorescence microscopy, the axonal marker NF—H was detected using mouse monoclonal antibody N52 (Sigma, Saint Louis, Mo.) or rabbit polyclonal antibody AB1989 (Chemicon); the axonal marker peripherin was detected using polyclonal antibody AB1530 (Chemicon); and the sheath marker myelin associated glycoprotein (MAG) was detected using an anti MAG monoclonal antibody (kindly provided by Dr. Elior Peles, Weizmann Institute). For detection of biotin, an anti biotin monoclonal antibody was used (Jackson ImmunoResearch, Baltimore, Pa.). Vimentin was detected using the anti vimentin monoclonal antibody MAB3400 (Chemicon).

Hippocampal and dorsal root ganglion (DRG) neurons were fixed by treatment with 3% paraformaldehyde, while control and injured sciatic nerve segments were fixed in 4% paraformaldehyde, frozen using Tissue-Tek and cross-sectioned (15 microns) using a Leica cryostat. Neuron cultures and sciatic nerve cross-sections were incubated with primary detection antibodies as indicated, followed by incubation with Rhodamine Red X-conjugated donkey anti rabbit and Cys-conjugated donkey anti mouse antibody (Jackson ImmunoResearch), prior to mounting in moviol (Calbiochem). Labeled neurons and sections were observed under an Olympus FV500 confocal laser scanning microscope (CLSM). For Rhodamine Red-X and Cy5 visualization, 543 nm and 633 nm wavelengths, respectively, were sequentially used.

Primary neuron cultures: Rat embryonic hippocampal culturing was performed as previously described (Brann, A. B. et al., 2002. J Biol Chem 277:9812-9818). Adult C57BL/6 mouse DRG neurons were cultured as previously described (Benson, C. J. et al., 1999. Circ Res. 84:921-928). Briefly, the ganglia were successively enzymatically dissociated in 100 units of papain (Sigma), a mixture of 10 mg collagenase-II (Worthington) and 12 mg dispase (Roche), followed by trituration in HBSS (pH 7.35) supplemented with 10 millimolar glucose and 5 millimolar HEPES. The cells were then recovered through a Percoll (Sigma) gradient, and were plated on poly-L-lysine-(Sigma) and laminin-(Invitrogen) coated cover-slips (BDH). The neurons were grown in F12 media (Gibco-BRL) for up to three days in an incubator at 37 degrees having a 5% carbon dioxide atmosphere.

NLS pull-down assays and co-immunoprecipitations: Control and lesioned nerve axoplasm samples (0.5 mg) were incubated overnight with 100 micromolar of either biotinylated nuclear localization signal (NLS)-containing peptide CTPPKKKRKV (SEQ ID NO: 1); where the NLS peptide per se is peptide PKKKRKV (SEQ ID NO: 2), or biotinylated reverse-NLS-containing peptide CTPVKRKKKP (SEQ ID NO: 3), where the reverse-NLS peptide per se is VKRKKKP (SEQ ID NO: 4), followed by a 2-hour incubation with streptavidin dynabeads (Dynal). All incubations were performed at 4 degrees centigrade. The beads were washed 3 times with nuclear transport buffer prior to elution of bound protein with 0.1% trifluoroacetic acid. The presence of importin-alpha4, importin-beta and dynein protein in the eluates were analyzed via Western immunoblotting analysis.

For co-immunoprecipitations, axoplasm from control or lesioned nerve was precleared for one hour with 80% Protein G-Sepharose (Amersham Bioscience) or protein A-Agarose (Roche), and incubated overnight with anti dynein antibody. The mixture was incubated with protein-G or Protein-A beads for 2 hours at room temperature. The protein-bound beads were pelleted, and washed 3 times with nuclear transport buffer or with phosphate-buffered saline solution. All steps were carried out at 4 degrees centigrade. Proteins bound to the beads were eluted by boiling, and the eluted protein was analyzed by western immunoblotting analysis using antibodies against importin-alpha4 and importin-beta.

Retrograde transport of NLS peptide in sciatic nerve: Seven microliter aliquots of 50 picomolar biotinylated NLS peptide or 50 picomolar biotinylated reverse NLS peptide were injected into sciatic nerve of anaesthetized rats using a Hamilton syringe concomitantly with a crush lesion distal to the injection site. Sciatic nerves were dissected out either immediately, or 6 hours after the treatment. Dissected nerves were processed for sectioning as described above and longitudinal 10 micron sections were prepared. Retrograde transport of the peptide was monitored by double immunostaining with Alexa Fluor 647-phycoerythrin-conjugated streptavidin (Molecular Probes) to visualize the biotinylated peptides and anti NFH antibody to visualize the axons.

Electrophoretic gel autoradiography: In order to evaluate whether importin-beta is synthesized in axons, sciatic nerve segments were incubated for 6 hours in Met/Cys-deficient DMEM medium (Gibco-BRL) containing 1 mCi/ml of [35]S-Met/Cys (Amersham Bioscience), with or without 10 micrograms/ml of the translation inhibitor cycloheximide (CHX, Sigma) or 5 micrograms/ml of the transcription inhibitor actinomycin D (ActD, Sigma), followed by extrusion of the axoplasm into nuclear transport buffer. The extruded axoplasm was immunoprecipitated with anti importin-beta antibody, and the immunoprecipitate was resolved via 10% SDS-PAGE. Following electrophoresis, the gel was dried and analyzed via autoradiography using FujiFilm BAS2500.

Quantitative analysis of adult DRG neuronal outgrowth: Adult DRG cultures were prepared as described, the cultures were grown for 24, 48 or 72 hours, fixed and then stained for the neuronal markers NF—H and peripherin Cover-slips with fixed and stained cultures were analyzed via laser-scanning fluorescence microscopy, and random fields were photographed from each treatment at ×20 magnification. The percentage of sprouting cells and neurite length in sprouting neurons were compared for the 48-hour cultures. NIH IMAGE 1.61/68k software was used for measuring cell body diameter and neurite length. Neurons displaying neurites twice the length of the cell body diameter were considered to have sprouted. Statistical analysis of results was performed using ANOVA.

In-situ hybridization analysis of importin-beta mRNA: In-situ hybridization was performed as previously described (Van Minnen J. and Bergman J J., 2003. Invert Neurosci. Jan. 25, 2003 on-line publication). For co-immunoprecipitations of MAPK by vimentin, 0.5 mg of axoplasm from control or lesioned nerve prepared as previously described was pre-cleared for one hour with 80% Protein G-Sepharose (Amersham ioscience), and incubated overnight with anti vimentin monoclonal antibody MAB3400 (Chemicon). The mixture was incubated with protein-G beads for 2 hours at 4 degrees centigrade. Following incubation, the protein-bound beads were pelleted and washed 3 times with nuclear transport buffer or phosphate-buffered saline solution. All steps were carried out at 4 degrees centigrade. Proteins bound to the beads were eluted by boiling, and the eluted protein was analyzed by western immunoblotting analysis using anti MAPK polyclonal antibody M7927 (Sigma) or anti activated MAPK monoclonal antibody M8159 (Sigma).

Importin-beta GST pulldown assays: Aliquots of 0.5 mg of axoplasm from control or lesioned nerve or of 1 mg pure vimentin (cytoskeleton) were pre-cleared for one hour with glutathione-conjugated sepharose 4B beads (Amersham Bioscience), and incubated overnight at 4 degrees centigrade with 5 mg purified glutathione S-transferase (GST)-conjugated importin-beta. The mixture was incubated with the GST-conjugated beads for 2 hours at 4 degrees centigrade. The protein-bound beads were pelleted, and washed 3 times with nuclear transport buffer or phosphate-buffered saline solution. All steps were carried out at 4 degrees centigrade. Proteins bound to the beads were eluted by boiling, and the eluted protein was analyzed by western immunoblotting analysis using anti vimentin monoclonal Ab as primary detection reagent.

Experimental Results:

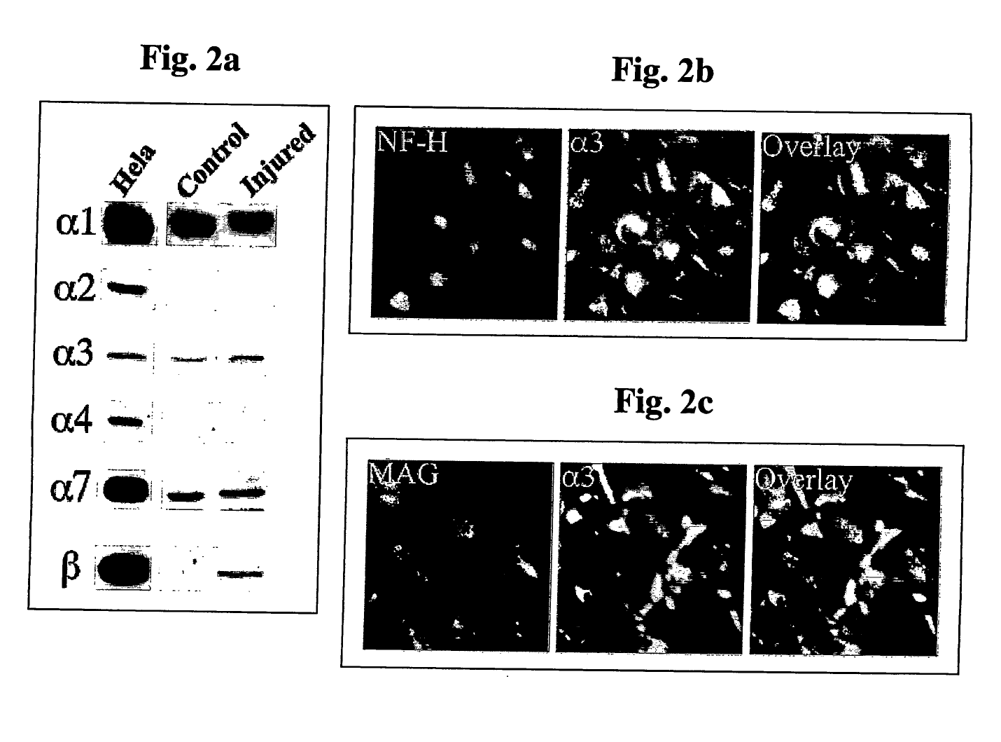

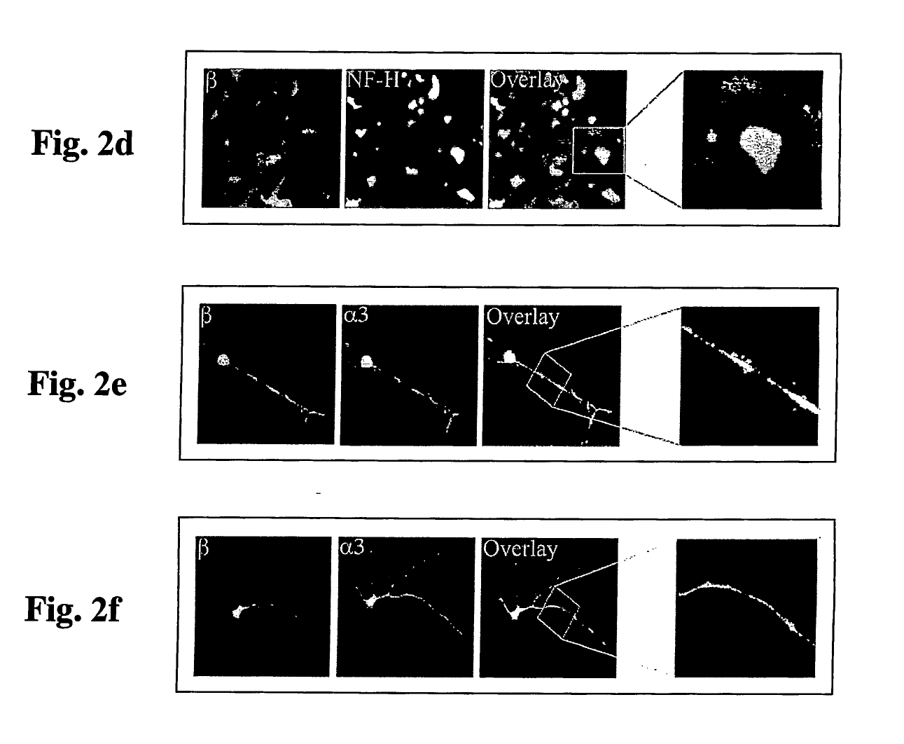

Importins are found in axons at a significant distance from neuronal cell bodies: Antibodies specific for human importin-alpha isoforms or for importin-beta were used for Western blot analysis of injured rat sciatic nerve. Nerves were dissected, incubated for designated periods, and axoplasm was then obtained by gentle squeezing of the nerve segments in physiological buffer. A number of importin-alpha family member proteins were found in sciatic nerve and their expression levels did not change significantly after lesions were induced, whereas, in striking contrast, significant amounts of importin-beta were observed only in axoplasm from lesioned nerves (FIG. 2a). In order to verify that the observed importins were indeed axonal, sciatic nerve cross-sections were co-immunostained with anti importin antibodies and antibody specific for the axonal or myelin sheath markers NF—H or MAG, respectively, and analyzed via immunofluorescence confocal microscopy. Importin-alpha4 (FIGS. 2b-c) and importin-beta (FIG. 2d) were found to specifically co-localize with the axonal marker in the sections. Similar results were obtained with importin-alpha1 and importin-alpha7 (data not shown). The distribution of importin-alpha4 and importin-beta was further evaluated in cultured adult DRG and hippocampal neurons. The growing axons of both well-differentiated DRG (FIG. 2e) and hippocampal (FIG. 2f) neurons were found to be immunopositive for both of these importin types.

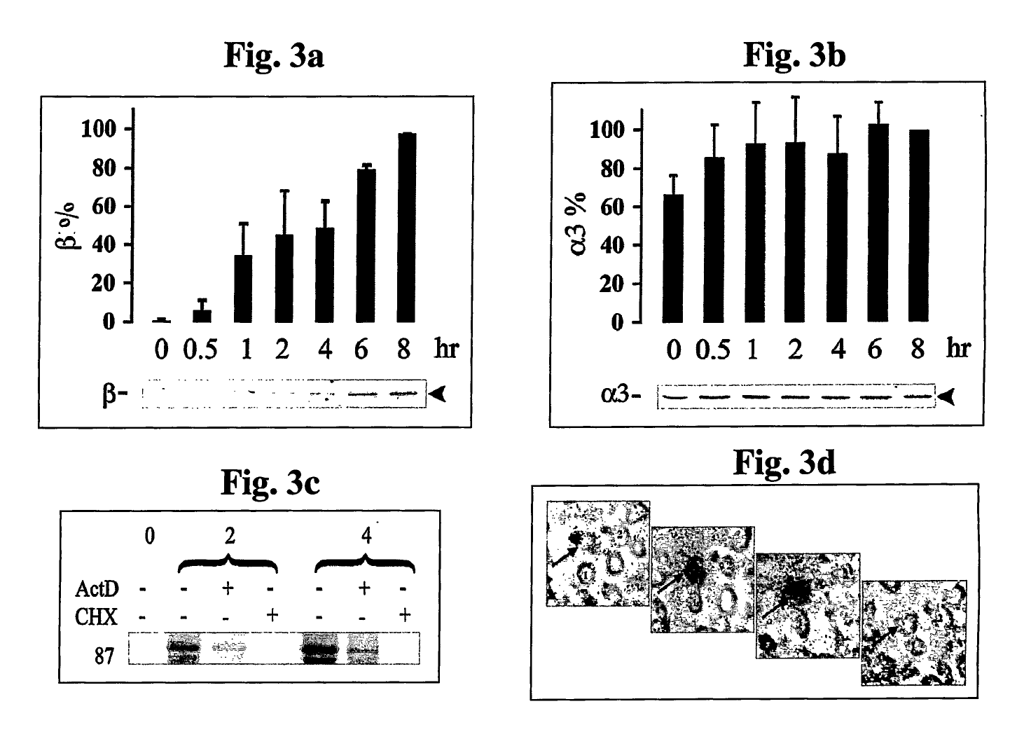

Expression of importin protein is up-regulated in lesioned axons: In order to assess the source and kinetics of appearance of importin-beta in lesioned nerve, segments of sciatic nerve were incubated in-vitro for various time periods following lesioning thereof, after which preparations of total axoplasmic protein were analyzed via Western immunoblotting analysis for importin-beta and importin-alpha4 protein content. As shown in FIG. 3a, a steady increase in the levels of importin-beta over time, for up to 8 hours was observed. In sharp contrast, levels of importin-alpha4 remained essentially stable in the nerve segments throughout the course of this experiment (FIG. 3b). Since the nerve segments incubated in-vitro did not contain any neuronal cell bodies, it was surmised that the increasing levels of importin-beta might arise from de-novo protein synthesis in the axons (Giuditta A. et al., 2002. Trends Neurosci. 25:400-4; Steward O., 2002. Cell 110:537-40). Incubation of lesioned sciatic nerve segments with radiolabeled amino acids revealed transcriptionally independent translation of importin-beta in the nerve axoplasm (FIG. 3c). In order to examine if importin-beta mRNA is indeed present in lesioned nerve axons so as to enable the observed de-novo importin-beta protein synthesis, in-situ hybridization analyses were performed on lesioned sciatic nerve axoplasm. Importin-beta-positive signal was indeed detected within axons in nerve cross-sections, as shown in FIG. 3d, thereby demonstrating the presence of importin-beta mRNA in lesioned nerve axoplasm. These results are supported by previous studies in which metabolic labeling of sciatic nerve segments and of isolated DRG axons revealed de-novo translation of importin-beta from preexisting mRNA (Zheng, J. Q. et al., 2001. J Neurosci. 21:9291-303).

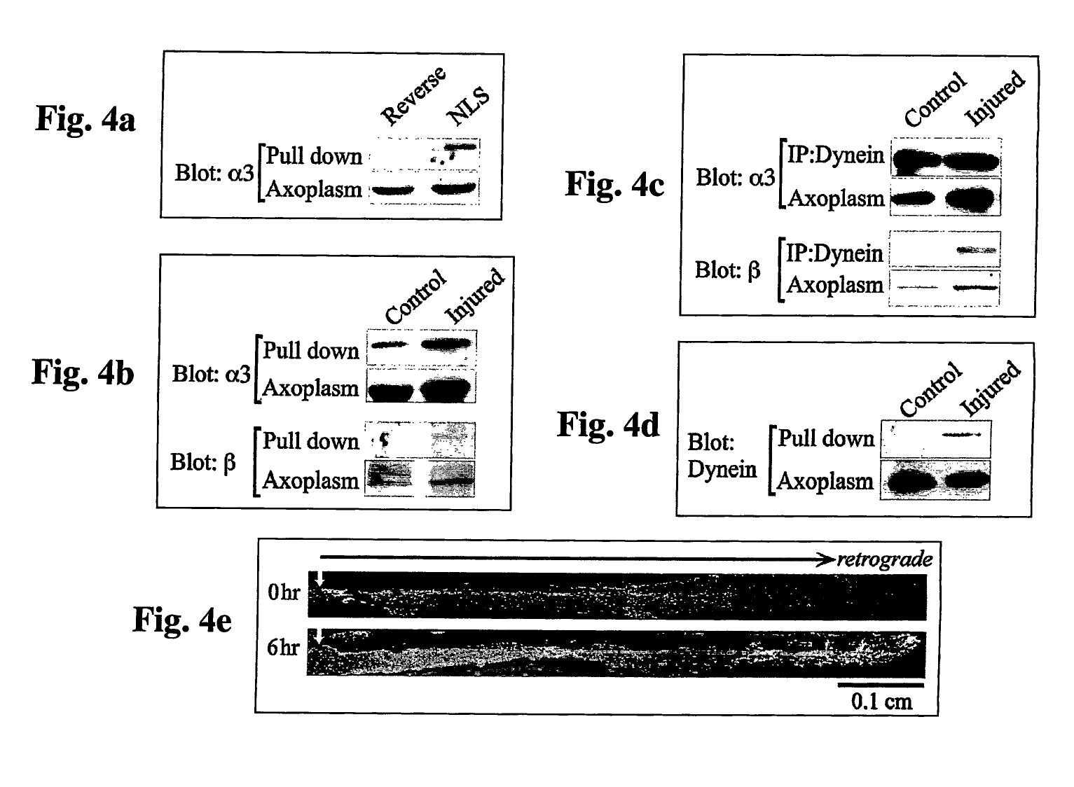

Formation of a high-affinity retrogradely trafficking NLS:importin-beta associated complex in lesioned axons: The experiments outlined above suggested that increased levels of importin-beta protein following lesioning might allow enhanced transport of NLS-bearing axonal proteins to the nucleus as a result of increased levels of functional complexes of such NLS-bearing proteins and importin-beta in the axoplasm of lesioned neurons. This possibility was examined by performing streptavidin pull-down assays on lesioned sciatic nerve axoplasm incubated with biotinylated NLS peptide or, as a negative control, biotinylated reverse-NLS peptide. Importin-alpha4 was found to co-precipitate with biotinylated NLS peptide but not with biotinylated reverse-NLS peptide (FIG. 4a), thereby confirming the specificity of the interaction. The amounts of importin-alpha4 and importin-beta co-precipitated with biotin-NLS from axoplasm of control versus injured nerves were then compared. As shown in FIG. 4b, there was a significant increase in the amount of importin-alpha precipitated from lesioned versus control nerve. Similarly, importin-beta increased from undetectable to detectable levels when comparing NLS precipitates from control versus lesioned nerve. The potential retrograde trafficking of NLS-importin complex in nerve axons was then examined by co-immunoprecipitation of importin-alpha4 and importin-beta with the retrograde motor protein dynein in axons. Importin-alpha4 co-precipitated with dynein from both control and injured nerves, whereas importin-beta was detectable only in co-immunoprecipitates from injured nerve (FIG. 4c). In accordance with this result, dynein was found to be associated with NLS peptide in NLS pull-down assays from injured but not control nerve (FIG. 4d). Finally, retrograde trafficking of complexes of NLS peptide and NLS-associated proteins was directly examined by microinjection of biotinylated NLS peptide into lesioned sciatic nerve in-vivo. Longitudinal sectioning of the nerve revealed retrograde trafficking of biotinylated NLS peptide (FIG. 4e), whereas no such transport could be visualized for the control biotinylated reverse-NLS peptide (data not shown).

NLS peptide competition delays the regenerative outgrowth of adult DRG neurons: After establishing the formation of an NLS-binding retrogradely trafficking complex in lesioned axons, the functional significance of this complex for neuronal regeneration was assessed by exposing triturated adult DRG neurons during trituration thereof to NLS peptide or reverse-NLS peptide, and culturing the triturated cells and monitoring regenerative outgrowth during a 3-day period. Trituration is the final mechanical dissociation procedure performed prior to plating the cells for in-vitro culture, and during this short step the neurons undergo mechanical axotomy, freeing the cell bodies for plating. The peptides were added to the trituration medium so as to be taken up at the site of injury to thereby compete with endogenous signaling proteins that would otherwise bind to the importin-containing complexes formed by the lesion. Following trituration, non-internalized peptides were removed by pelleting the neurons through a Percoll cushion prior to plating the cells. As shown in FIG. 5a, after 48 hours of culture a clear inhibition in neurite outgrowth was observed in NLS peptide-treated cells, whereas cells treated with reverse-NLS peptide were indistinguishable from non-treated neurons. The DRG culture is composed of 2 types of cell populations; larger neurons (mechanoreceptors) which are mostly stained with the NF—H marker and smaller ones (nociceptors) mostly stained by peripherin. The inhibition was observed both in larger NFH-positive and in smaller peripherin-positive neuronal populations. Quantification of the effect of NLS peptide on regenerative outgrowth of neurons after 48 hours in culture revealed a significant reduction in the percentage of regenerating cells (FIG. 5b), and a highly significant reduction in neurite length in the regenerating cells (FIG. 5c).

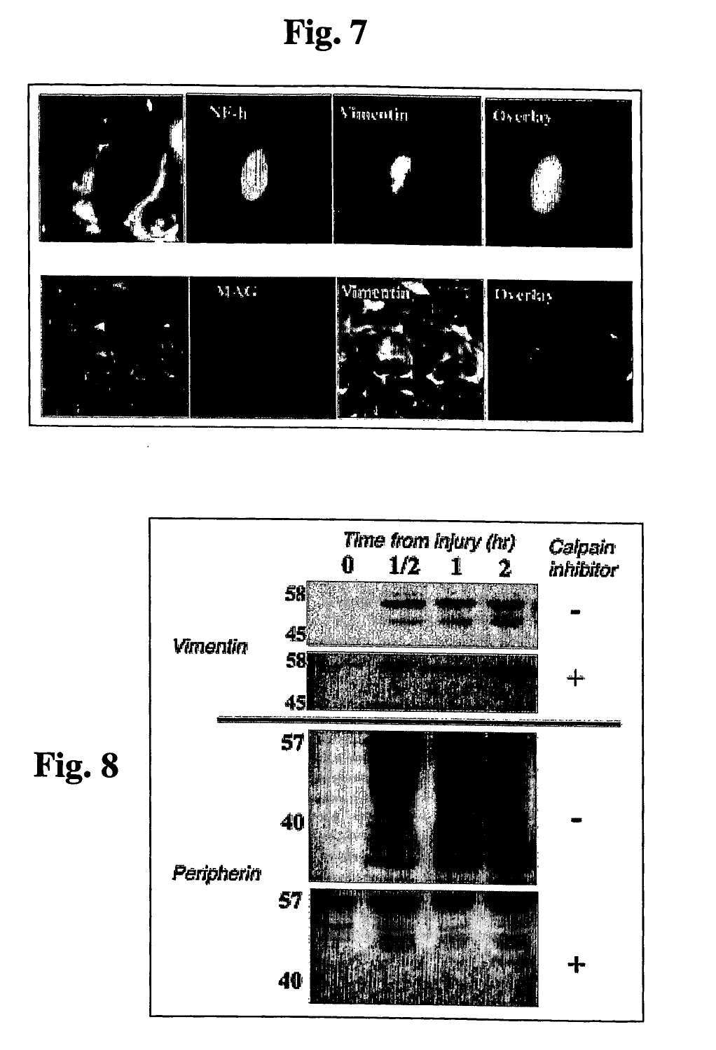

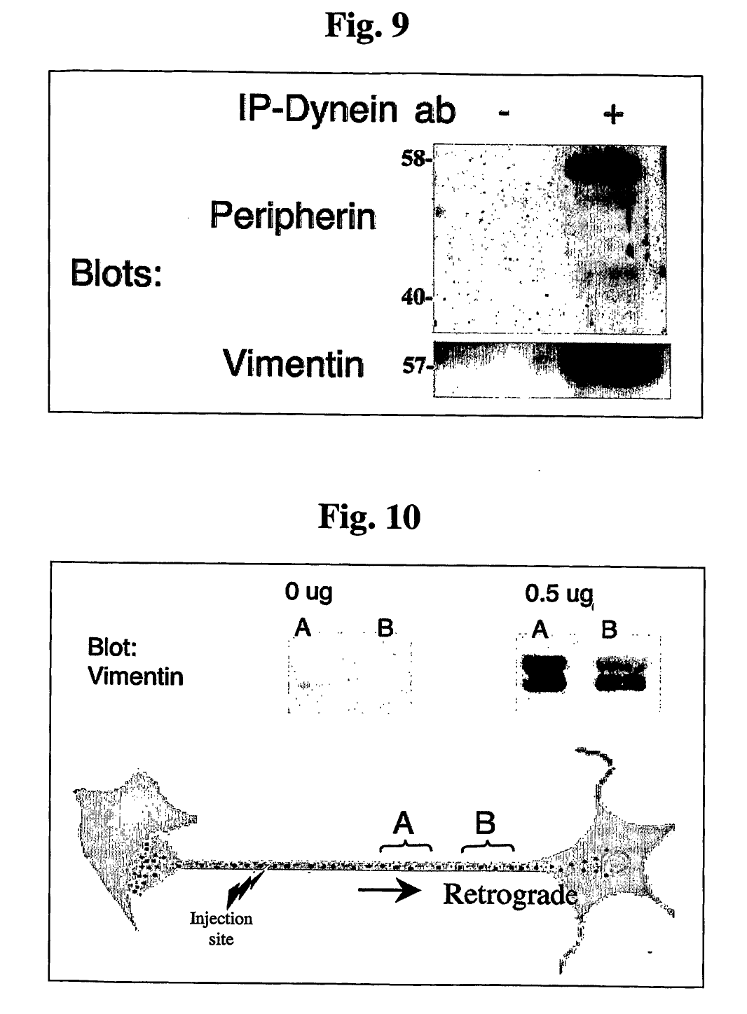

In order to identify signaling proteins that may retrogradely traffic as part of the complex comprising NLS, importin-beta, importin-alpha4, and dynein described above, a number of candidate molecules known to be upregulated in sciatic nerve axoplasm following nerve injury, such as the type III intermediate filament proteins vimentin and peripherin were tested. Vimentin was found to be expressed in a subset of adult rat sciatic nerve injured axons (FIG. 7). Soluble vimentin or peripherin monomers or their calpain cleavage products were not found in axoplasm extracts of non-injured nerve. Injury induces synthesis and/or cleavage of the intermediate filaments allowing them to interact with dynein-containing complex, but within 30 minutes following axonal lesion, soluble monomeric vimentin and peripherin and truncation products thereof appeared in sciatic nerve axoplasm and levels of these soluble intermediate filament products increased with time (FIG. 8). Concomitantly, an enhanced interaction of soluble vimentin and peripherin with a dynein-containing complex was observed by co-immunoprecipitation of these proteins with axoplasmic dynein (FIG. 9). Finally, injection of exogenous vimentin into injured sciatic nerve resulted in retrograde transport of the injected vimentin towards cell bodies (FIG. 10). Thus, vimentin/peripherin monomers and truncation products are released into the fluid compartment of the axoplasm upon injury, where they are recruited into a retrograde complex trafficking on dynein.

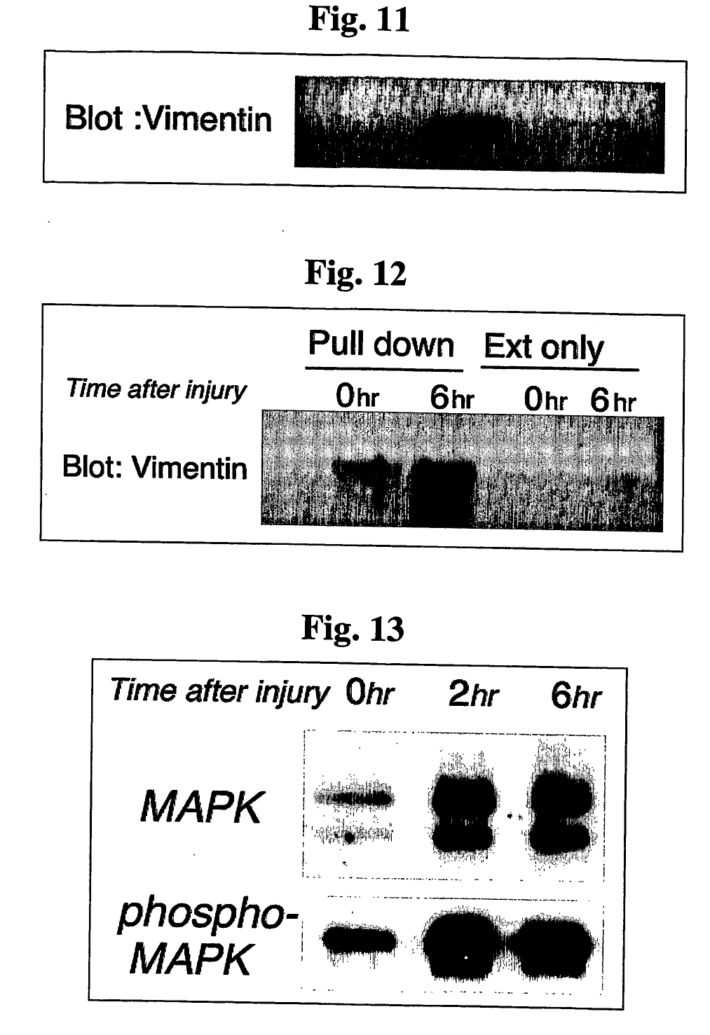

As described above, a retrogradely transported complex is formed in lesioned axons by recruitment of importin-beta protein to a complex comprising NLS peptide, importin-alpha4, and dynein. The possibility of a specific binding interaction between vimentin and NLS in this complex was investigated via co-precipitation studies which revealed that the interaction of vimentin with dynein is not competed by excess NLS peptide. The possibility direct interaction between vimentin and importin-beta in this complex was therefore examined, and was indeed shown to occur by analyses involving co-precipitation of a GST-importin-beta fusion protein with vimentin in-vitro (FIG. 11), or with endogenous vimentin in axoplasm from injured nerve (FIG. 12).

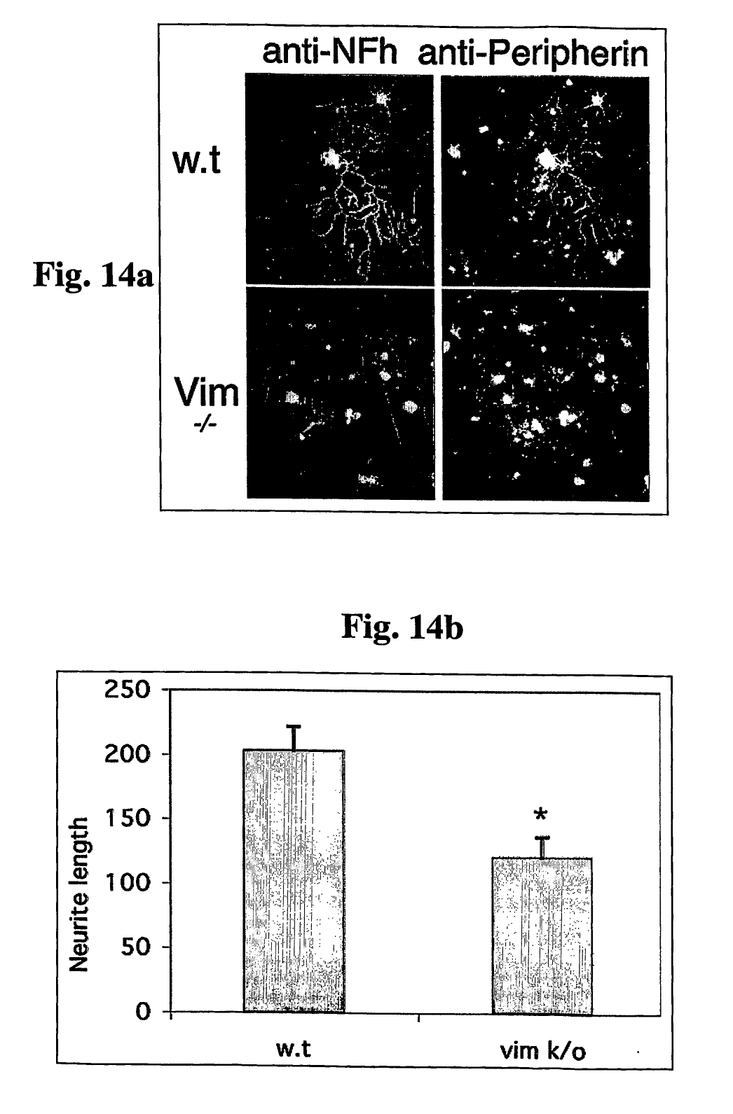

Since, as described above, vimentin binds directly to importin-beta, and interacts with the protein complex retrogradely transported via dynein, the possibility that vimentin acts as a carrier of signaling molecules was tested. Analysis via co-immunoprecipitation and GST-pull down assay indeed revealed a strong binding of MAP kinases and phosphorylated MAP kinases (specifically ERK1 and ERK2) to vimentin in injured nerve axoplasm (FIG. 13). The specific association of vimentin and MAP kinases was further demonstrated by co-precipitation of these molecules in-vitro. Thus, type III intermediate filament proteins, in particular vimentin, provide an accessory binding scaffold by which signaling proteins lacking an NLS can access the retrograde transport machinery in injured nerve. The functional importance of this component for regenerative growth signaling was assessed by examining the regenerative outgrowth of adult DRG neurons from vimentin-null mice. As shown in FIG. 14a, there was reduced outgrowth of both NFH-positive and peripherin-positive neurons in vimentin-null mice, and as shown in FIG. 14b, there was reduced outgrowth in vimentin-null mice as determined via neurite length following outgrowth.

Thus, taking all the data together, vimentin is up-regulated in sciatic nerve axoplasm after nerve injury, interacts with the importin-dynein retrograde trafficking complex via a direct interaction with importin-beta, thereby providing a link for signaling molecules such as MAP kinases to the retrograde transport machinery. The fact that neurons from vimentin-null mice show impaired regenerative outgrowth suggests that this mechanism is important for functional neuronal regeneration following nerve injury.

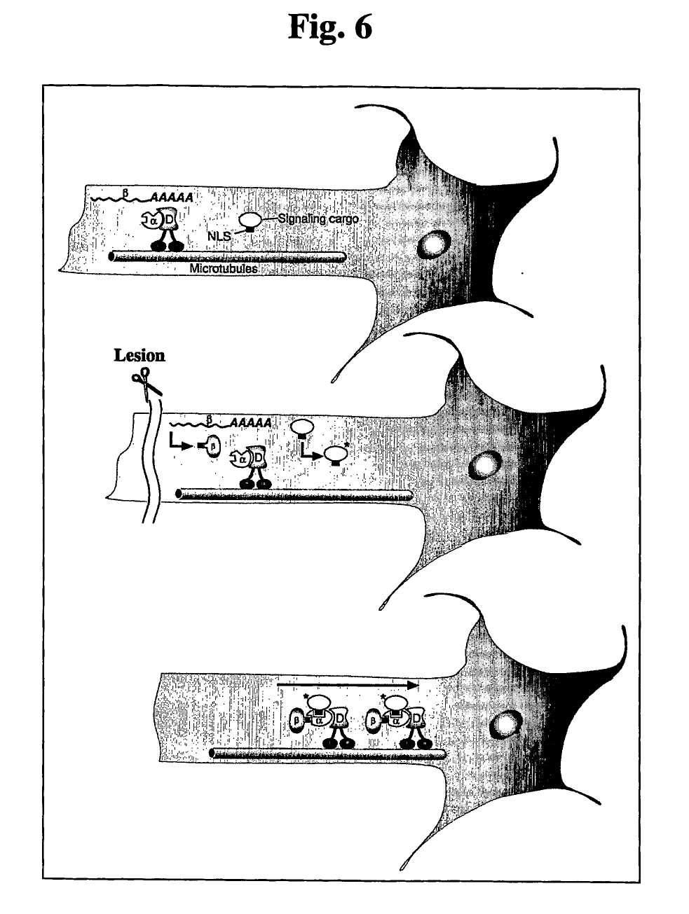

An importin-targeted retrograde injury-signaling complex in lesioned nerve axons: Thus, the above-described results demonstrate that an importin mediated signal participates in the regeneration response of axotomized adult DRG neurons, and that competition of this signal delays neuronal regeneration. A schematic model depicting the formation of such a signaling complex is shown in FIG. 6. Two critical lesion-induced events are postulated to occur in parallel: (i) local synthesis of importin-beta triggering formation of a high affinity NLS-binding complex associated with dynein; and (ii) modification of NLS-bearing proteins that associate with the complex as a signaling cargo (Ambron R T. and Walters E T., 1996. Molecular Neurobiology 13:61-79; Schmied R. and Ambron R T., 1997. J Neurobiol. 33:151-60). This mechanism appears to play a major role in signaling the regeneration response in peripheral sensory neurons (Blesch, A., and M. H. Tuszynski. 2004. Nat Med. 10:236-7). Hence, an important aspect of the model described in FIG. 6 is that it provides for the first time a satisfactory framework for systematic work on intrinsic regeneration mechanisms.

A variety of kinases have been suggested to act as survival or regeneration enhancing retrograde signals (Watson F L. et al., 2001. Nat Neurosci. 4:981-8; Ginty D D., and Segal R A., 2002. Curr Opin Neurobiol. 12:268-74), hence phosphorylation is a likely mechanism for activation of retrograde signaling proteins. Signaling proteins carried as cargo on the imports might also arise from local axonal synthesis. Recent studies have indicated that local protein synthesis plays a role in axonal pathfinding, allowing a physiological response at the site of synthesis (Campbell, D S. and Holt C. E., 2001. Neuron 32:1013-26; Brittis P A. et al., 2002. Cell 110:223-35). The presently described data reveals a way in which maintenance of a latent signaling complex in the form of specific axonal mRNAs can allow regulated long-range signaling to the cell body by local protein synthesis of critical components.

Although significant progress has been made with regards to understanding the structure and mode of action of the importins in nucleocytoplasmic transport (Chook Y. and Blobel G., 2001. Curr Opin Struct Biol. 11:703-15), less attention has been focused on physiological and developmental roles of these molecules. The presently described data indicate that importins have important roles in long-range retrograde transport of axonal signals, specifically in the context of nerve injury. However it should be noted that cytoplasmic roles for importins might not be restricted solely to injury situations, and importins were recently shown to act as cytoplasmic chaperones for very basic proteins that are otherwise prone to aggregation (Jakel, S. et al., 2002. EMBO J. 21:377-86). Defects in development have been described in importin-null mutants in both C. elegans and Drosophila (Mathe E. et al., 2000. Dev Biol. 223:307-22; Kumar, J P. et al., 2001. Dev Biol. 240:315-25; Geles, K G. and Adam, S A., 2001. Development 128:1817-30). Most strikingly, the fly importin-beta mutant reveals defects in guidance and cell adhesion of specific photoreceptor neurons, indicating that axon guidance in the eye is particularly dependent on importin-beta (Kumar, J P. et al, 2001. Dev Biol. 240:315-25). This may be due to perturbation of importin-beta-dependent retrograde signaling in the axons of fly photoreceptor neurons. Indeed, the presence of importin-beta mRNA in axons, and the localized regulation of importin-beta protein by de-novo synthesis may provide a versatile mechanism for regulating retrograde signaling in both normal and injured neurons. In essence, any signal that stimulates local synthesis of importin-beta will lead to local formation of an NLS-binding retrogradely transported complex, thus importins might be fundamental enabling components for a rich spectrum of intraaxonal signals. As presently demonstrated for the first time, both importin-alpha and importin-beta are observed throughout dendrites and axons of cultured embryonic hippocampal neurons (FIG. 2f). Synaptic activation of hippocampal neurons has been shown to trigger translocation of these nuclear transport factors from terminal to nucleus (Chen, D. Y. et al., 2003. Society for Neuroscience Abstracts. 471.7). Other studies have shown retrograde transport of the NLS-containing transcription factor NF-kB following synaptic activation of hippocampal neurons (Meffert, M. K. et al., 2003. Nat Neurosci. 6:1072-8; Wellmann, H. et al., 2001. J Biol Chem. 276:11821-11829). Taken together, these studies strongly support diverse roles for importins in different facets of neuronal signaling and function.

Summary: The presently described results unexpectedly revealed critical mechanisms involved in mediating retrograde transport in mammalian neurons enabling regulation of neuronal regeneration and delivery of exogenous compounds to neuronal cell bodies via such transport mechanisms. Namely, the presently described results unexpectedly revealed that: (i) importin-alpha proteins are found throughout neuronal axons, at significant distances from the cell body; (ii) importin-beta protein is synthesized de-novo throughout neuronal axons, at significant distances from the cell body in injured axons, but is not present in non-injured, axons; (iii) formation of NLS:importin-alpha4 complex significantly increases in axons following injury thereof; (iv) soluble intermediate filament protein monomers, such as vimentin and peripherin, specifically accumulate in injured, as opposed to non-injured, axons and specifically complex therein with activated ERK1 and ERK2; (v) importin-beta directly binds to such intermediate filament protein monomer:ERK1/2 complex, such NLS:importin-alpha4 complexes, and the retrograde motor protein dynein to form a retrogradely transported complex comprising such components, as demonstrated in injured axons; and (vi) formation of such a retrogradely transported complex is positively correlated with axonal growth, in particular regenerative axonal growth following axonal injury.

Conclusion: Thus, the present results demonstrate that lesion-induced up-regulation of axonal importin-beta protein synthesis drives the formation of a retrogradely transported injury-signaling complex in damaged nerve. Such findings can be directly exploited in numerous ways. In particular, regulation of levels of the aforementioned retrograde complex constituents, in particular those of importin-beta protein can readily be employed for up- or down-regulating retrograde transport, and thereby for regulating processes such as neuronal regeneration. Also, compounds such as diagnostic or therapeutic compounds, nucleic acids, viruses, etc., can be attached to such newly revealed retrograde transport complex constituents, and such cargo can thereby be associated with the importin-beta containing retrograde transport complex to be retrogradely delivered to neuronal cell bodies. Such an approach is particularly advantageous for delivering cargo to inaccessible locations in the central nervous system such as the brain. The aforementioned retrograde transport up-regulatory method and exogenous compound delivery method enabled by the presently described results can furthermore be used in combination to achieve optimal retrograde cargo delivery. Such an approach can be used, for instance, for delivering chemotherapeutic agents via axonal retrograde transport for targeting malignancies with known neurotropisms, especially during metastatic stages of such malignancies. Hence, the presently described method can be used for optimally diagnosing and treating numerous nervous system diseases.

### Example 2

### Vimentin Binding to Phosphorylated ERKs Enables Calcium-Dependent Dephosphorylation-Protected Transport of Activated MAP Kinases in the Retrograde Injury-Signaling Complex of Sensory Axons: Novel Calcium Regulatable Molecular Switch for Controlled Retrgrade Delivery of an Active Kinase or of a Cargo Complexed Therewith

Background: Neuronal retrograde injury signals must traverse long distances from the axonal lesion site to the cell body while maintaining the integrity of the signaling moiety. Phosphorylated kinases have been proposed to participate in such signaling, however the mechanism underlying their transport and how they preserve their phosphorylation en route has not been elucidated. Previous studies have suggested that a MAP kinase ERK1/2 homolog is a retrograde injury signal in Aplysia neurons (Sung et al., 2001. J Neurobiol 47, 67-79), however the only available Aplysia MAPK sequence lacks an obvious NLS sequence. Moreover, nuclear import of different ERK family members occurs via mechanisms that are not dependent on classical NLS binding to importin-alpha/-beta heterodimers (Ferrigno et al., 1998. Embo J 17, 5606-5614; Lorenzen et al., 2001. Development 128, 1403-1414; Matsubayashi et al., 2001. J Biol Chem 276, 41755-41760; Whitehurst et al., 2002. Proc Natl Acad Sci USA 99, 7496-7501). Taken together, these reports suggest an NLS-independent retrograde injury signal pathway for MAP kinases. Differential proteomics analysis in Lymnaea nerve has identified soluble cleavage products of a Lymnaea intermediate filament protein as a major component of the retrograde protein ensemble after lesion (Perlson et al., 2004. Mol Cell Proteomics 3, 510-520). This was intriguing in light of the fact that truncation mutants of the closely related mammalian type III intermediate filament vimentin can translocate from cytoplasm to nucleus in transfected cells (Lowrie et al., 2000. J Struct Biol 132, 83-94; Rogers et al., 1995. Eur J Cell Biol 66, 136-150). Moreover, vimentin is known to interact with a number of signaling molecules (Paramio and Jorcano, 2002. Bioessays 24, 836-844). Thus, while reducing the present invention to practice, the present inventors analyzed the function of phosphorylated MAP kinase and mammalian type III intermediate filaments in retrograde injury signaling and unexpectedly uncovered that vimentin binding to phospho-ERKs enables calcium-dependent dephosphorylation-protected transport of activated MAP kinases in the retrograde injury-signaling complex of sensory axons. As such, while conceiving the present invention, the present inventors hypothesized that such mechanisms could be harnessed to enable a calcium regulatable molecular switch for controlled delivery of an active kinase or of a cargo complexed therewith, in cells such as neurons.

Materials and Methods:

Animals, in-vivo procedures and cultures: Adult (8-12 weeks old) male Wistar rats were purchased from Harlan Inc. (Rehovot, Israel). Wild-type or vimentin^{-/-} (vimentin-null) SV129 mice (provided by Dr. E Colucci-Guyon, Pasteur Institute) were bred and maintained at the Veterinary Resources Dept of the Weizmann Institute, as previously described (Colucci-Guyon et al., 1994). Sciatic nerve crush was performed as previously described (Hanz et al., 2003. Neuron 40, 1095-1104). Axoplasm was obtained from freshly dissected nerves after gentle compression in phosphate-buffered saline (PBS) or in nuclear transport buffer (NTB, Hanz et al., 2003. Neuron 40, 1095-1104) containing protease inhibitors (Roche) and 1 millimolar orthovanadate when necessary. Purity of axoplasm was verified by Western blotting for Schwann cell (S-100) and nuclear (RCC1) markers as previously described (Hanz et al., 2003. Neuron 40, 1095-1104). DRG cultures from adult animals were as previously described (Hanz et al., 2003. Neuron 40, 1095-1104). Vimentin trituration into DRGs was carried out by introducing 100 micrograms of vimentin to the trituration medium for 15 ganglia. Vimentin was prepared for trituration by cleavage with calpain (100 microunits; 2 hours). In-vivo conditioning of sciatic nerve lesions was carried out as previously described (Hanz et al., 2003. Neuron 40, 1095-1104), and cultures from L4-L5 DRGs were set up three days after the conditioning crush. Cultures were fixed after 18 hours and length of the longest axon per neuron was measured. All animal experimentation was carried out only after approval by the Weizmann Institute IACUC, and with strict adherence to IACUC guidelines for minimization of animal usage and suffering.

Antibodies, Western Blots, and Immunofluorescence: The following antibodies were from Chemicon International (Temecula, Calif.): monoclonal anti-vimentin clone V9 MAB3400; polyclonal anti-vimentin antibody AB1620; polyclonal anti-peripherin antibody AB1530; monoclonal anti-dynein 74 kDa intermediate chain antibody MAB1618; monoclonal anti-beta-galactosidase antibody AB1802; polyclonal anti-beta-galactosidase antibody AB1211; anti-NFH polyclonal antibody AB1989. From Sigma (Rehovot, Israel): monoclonal anti-NFH antibody clone N52; polyclonal anti-ERK antibody M5670; monoclonal anti-phospho-ERK antibody M8159. The importin-beta monoclonal antibody 3E9 MA3-070 was from Affinity Bioreagents (Golden, Colo.), polyclonal anti-beta-tubulin antibody SC 9104 was from Santa Cruz (Santa Cruz, Calif.), monoclonal anti-phosphorylated Elk-1 (pElk-1) antibody #9186 was from Cell Signaling (Beverley, Mass.), and anti-importin-alpha4 antibody was a kind gift from Dr. Karsten Weis (U.C. Berkeley, USA). Horseradish peroxidase (HRP)-conjugated secondary antibodies were from Bio-Rad; and fluorescent secondary antibodies were from Jackson ImmunoResearch. Western blots and immunostainings were carried out as previously described (Hanz et al., 2003. Neuron 40, 1095-1104). For Western blots, axoplasm proteins were resolved via 10% SDS-PAGE, the gels were transferred to nitrocellulose and after reaction with the desired antibodies were developed via enhanced chemiluminescence (ECL; Pierce). For immunofluorescence analysis, DRG neurons were fixed with 3% paraformaldehyde, while control and injured sciatic nerve segments were fixed with 4% paraformaldehyde, frozen with Tissue-Tek (Sakura, Tokyo) and sectioned longitudinally at 10 micron thickness using a Leica cryostat. Neuron cultures and sciatic nerve sections were mounted in moviol (Calbiochem) and observed under an Olympus FV500 confocal laser scanning microscope. For Rhodamine RED-X and Cy5 visualization 543 nm and 633 nm wavelengths in a sequential manner were used.

Recombinant expression of pERK and importin-beta: To obtain doubly phosphorylated ERK (pERK), activated human GST-ERK2 was co-expressed with constitutively active MEK1 in BL21 bacteria, as previously described (Wilsbacher and Cobb, 2001. Methods Enzymol 332, 387-400). The bacteria were grown in 2YT medium at 30 degrees centigrade to an optical density of 0.6, and then 1 millimolar IPTG was added for an additional four hours. GST-importin-beta and His-tagged importin-beta constructs were kindly provided by Dr. Steve Adam (Northwestern University, Chicago) and Dr. Ziv Reich (Weizmann Institute), respectively. Importin-beta constructs were amplified in BL21 bacteria cultured in LB medium at 37 degrees centigrade until OD 0.5, followed by overnight incubation with 100 micromolar IPTG at 26 degrees centigrade. Proteins were purified over Glutathione-Sepharose 4B (Amersham Biosciences) or Nickel-NTA Agarose (Qiagene) according the manufacturer's instructions.

Pull-down assays and co-immunoprecipitations: Sciatic nerve axoplasm (500 micrograms) was pre-cleared for 1 hour with 80% protein G-Sepharose (Amersham Bioscience). Following overnight incubation with primary antibody, complexes were incubated on protein-G beads for 2 hours, washed extensively and eluted by boiling in SDS-PAGE sample buffer before loading on gels for Western blot analysis. For co-immunoprecipitation in the presence of competitors, 2 micromolar NLS (SEQ ID NO: 2) or reverse-NLS (SEQ ID NO: 3) peptide was added to the axoplasm for overnight incubation at 4 degrees centigrade. For direct pull-downs, GST-pERK (0.5 microgram) or importin-beta (1 microgram) was equilibrated in PBS or NTB and added to 500 microgram aliquots of axoplasm from different post-lesion times. Direct interactions were tested in-vitro by mixing 0.1 microgram recombinant Syrian hamster vimentin or neurofilament (Cytoskeleton Inc., Denver, Colo.) to GST-pERK or GST-importin-beta and incubated for 2 hours at 37 degrees centigrade. All pull-downs were washed twice with 0.2 molar NaCl in PBS or NTB and twice again with PBS or NTB before elution in SDS-PAGE sample buffer for loading on gels.

Phosphatase protection assay: His-Imp-beta (1 microgram) was incubated with pERK (0.5 microgram) and with vimentin or NFH (0-2 micrograms) for 2 hours at 37 degrees centigrade. Alkaline phosphatase (AP; 2 units; Roche, Mannheim, Germany) or axoplasm from injured nerve (100 micrograms) was then added for an additional 30 minutes. Complexes were purified over Nickel-NTA-Agarose followed by Western blot analysis for pERK. Alkaline phosphatase activity was verified with the synthetic substrate p-nitrophenyl phosphate (pNPP, Catalog # S0942, Sigma) dissolved in 0.1 molar glycine buffer (0.1 molar glycine, 1 millimolar MgCl₂, 1 millimolar ZnCl₂; pH 10.4) for a stock solution of 1 mg/ml. One unit AP was added to a reaction mixture consisting of 1 microgram pNPP with 2 micrograms vimentin or NFH in Tris buffer pH-7.5, and incubated for 5, 15, and 30 minutes before stopping the reaction with 3N NaOH. The yellow hydrolysis product was quantified spectrophotometrically at 405 nm.

Retrograde movement: Rat sciatic nerves were crushed and dissected out at increasing times after lesion (0-24 hours). Dissected nerves were divided into consecutive segments of about 10 mm each before extraction of axoplasm. Afterwards, 300 micrograms of axoplasm from each segment was immunoprecipitated with dynein antibody and precipitates were analyzed by Western blot for vimentin and pERK. Follow-up experiments in mouse were conducted with wild-type or vimentin^{-/-} sciatic nerves, crushed and ligated as described. After 16 hours the nerves were dissected, fixed, and sectioned longitudinally over the ligation as previously described (Hanz et al., 2003. Neuron 40, 1095-1104). Sections were then immunostained for pERK and NFH.

Phosphorylated Elk-1 response: Rat L4-5 DRGs were dissected 0-24 hours after sciatic nerve lesion into lysis buffer (50 millimolar Tris-HCL pH 7.4; 150 millimolar NaCl; 1 millimolar PMSF; 1 millimolar EDTA; 1% Triton X-100 and protease inhibitors cocktail [Roche]), and processed for Western blot of pERK and phosphorylated Elk-1 (pElk-1). For pElk-1 analysis in mouse, DRG processes were lesioned at about 1 mm distance from the ganglia before transfer to PBS and incubation in-vitro for up to one hour at 37 degrees centigrade, followed by lysis and Western blot analysis.

Experimental Results:

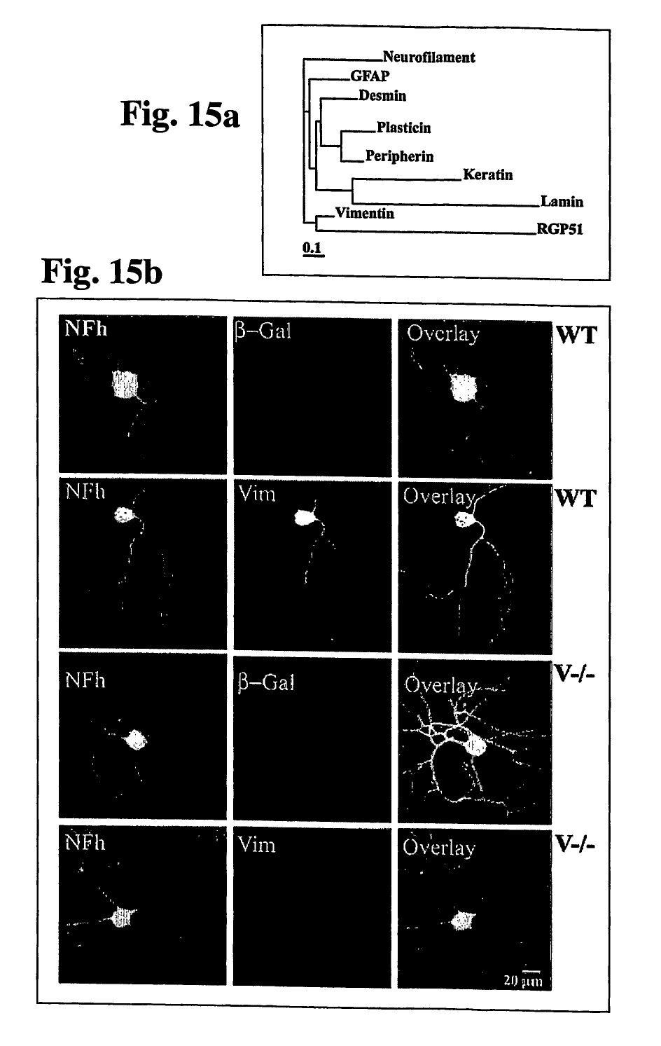

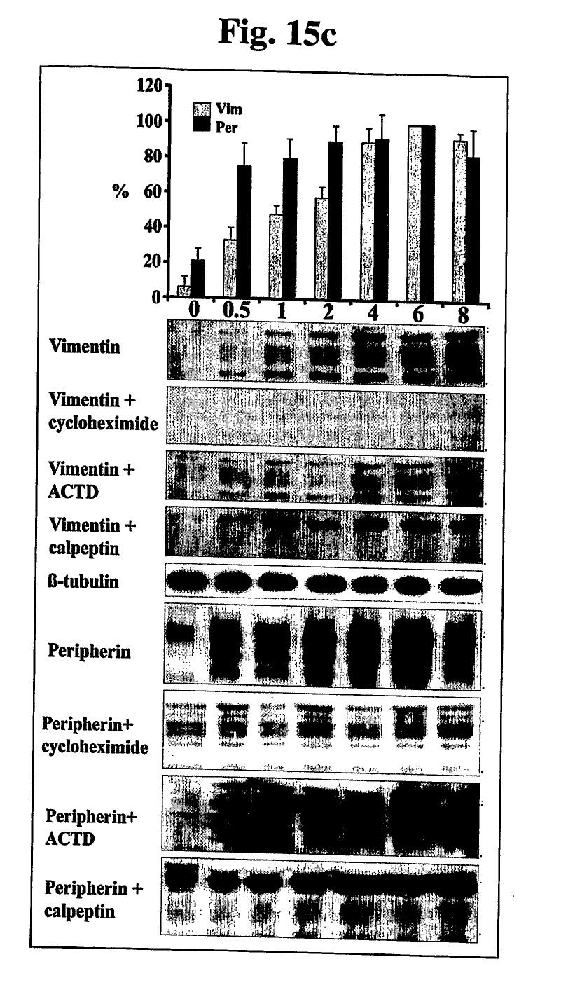

The RGP51 molluscan intermediate filament identified via proteomics screening was found to be most homologous to the mammalian type III intermediate filament vimentin (FIG. 15a). In the nervous system, vimentin is most prominently expressed in glial cells (Evans, 1998. Bioessays 20, 79-86; Gimenez y Ribotta et al., 2000. Glia 31, 69-83; Menet et al., 2003. Proc Natl Acad Sci USA 100, 8999-9004), but it is also expressed in neurons early in development and following injury, where it has been suggested to play a role in early generation and extension of neurites Boyne et al., 1996. Int J Dev Neurosci 14, 739-748; Shea, 1990. Brain Res 521, 338-342; Shea et al., 1993. J Neurosci Res 36, 66-76). Peripherin is another closely related type III intermediate filament that is highly expressed in peripheral nerve axons and upregulated by injury (Lariviere and Julien, 2004. J Neurobiol 58, 131-148). The possibility was therefore examined that vimentin or peripherin might be mobilized in mammalian nerve in a similar manner to that previously observed for RGP51 in Lymnaea (Perlson et al., 2004. Mol Cell Proteomics 3, 510-520). First, vimentin expression in cultured DRG neurons from adult mouse was examined. As shown in FIG. 15b, vimentin immunostaining was observed in the cell body and processes of regenerating NFH-positive sensory neurons in the culture. Specificity of the neuronal immunostaining was confirmed by complete lack of staining in neurons from vimentin^{-/-} mice. Since the mouse line was engineered by inserting beta-galactosidase (beta-Gal) into the vimentin locus (Colucci-Guyon et al., 1994. Cell 79, 679-694), beta-Gal staining served to confirm locus re-expression after neuronal lesion (FIG. 15b). Overall, an average of 64% of the NFH positive neurons were also positive for vimentin in wild-type cultures, and 58% of NFH-expressing vimentin-null neurons were positive for beta-Gal. The occurrence of both type III intermediate filaments in sciatic nerve axoplasm was then examined in-vivo. Little or no intermediate filament was found in axoplasm before injury, but within 30 minutes of a lesion the appearance of soluble forms of both vimentin and peripherin was observed (FIG. 15c). Levels of these proteins increased during eight hours following injury, and it was possible to block the increase with inhibitors of translation, but not of transcription. Multiple bands were observed for both vimentin and peripherin, and the lower molecular masses disappeared upon treatment with the calpain inhibitor calpeptin. Thus, soluble forms of vimentin and peripherin are produced in injured sciatic nerve axoplasm by a combination of local translation and calpain cleavage activity.

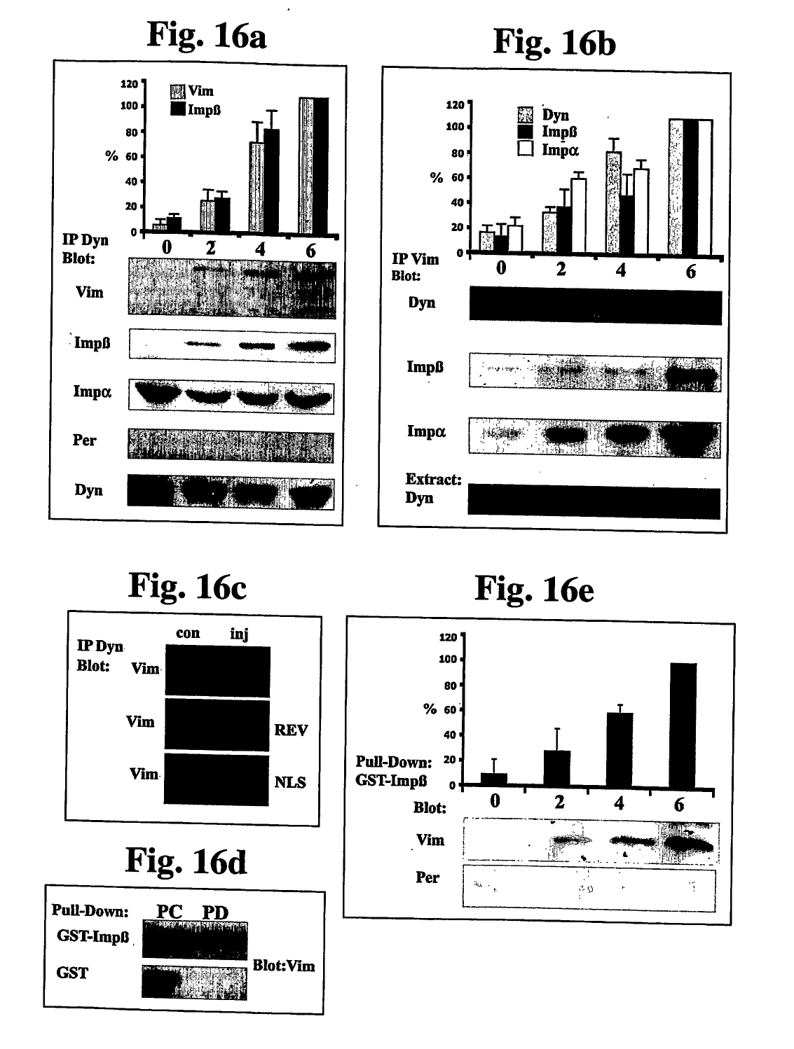

Co-immunoprecipitation of vimentin or peripherin with the dynein motor complex in injured nerve was then examined. Vimentin was observed to co-precipitate with dynein in increasing amounts with time after injury (FIG. 16a). In contrast, peripherin was not found in any of the precipitates, despite the high levels of peripherin found in axoplasm at these time points (see FIG. 15c). As reported previously, importin-alpha4 was found in the complex in both uninjured and lesioned nerve, while importin-beta levels increased in the co-precipitate after injury (FIG. 16a). Reciprocal co-immunoprecipitations with an antibody directed against vimentin revealed increasing amounts of dynein, importin-alpha and importin-beta with time after injury (FIG. 16b). As described above and in previous studies (Hanz et al., 2003. Neuron 40, 1095-1104), the present inventors showed that a functional NS-binding complex is formed by recruitment of importin-beta to importin-alpha complexed with dynein in lesioned axons. The issue of whether vimentin was accessing the NLS binding site in this complex was addressed by conducting co-precipitation experiments of dynein with vimentin in the presence of excess NLS peptide or reverse-NLS peptide as control. As shown in FIG. 16c, the interaction of vimentin with dynein is not competed by excess NLS. Moreover, vimentin co-precipitates from axoplasm together with the importins in NLS pull-downs (data not shown), indicating that a vimentin-importin interaction cannot be mediated via the NLS-binding site. A number of non-classical importin cargoes undergo nuclear uptake by direct binding to importin-beta (Cingolani et al., 2002. Mol Cell 10, 1345-1353). Experiments were therefore performed to test for a direct interaction of vimentin with importin-beta by in-vitro co-precipitation of vimentin with a GST-importin-beta fusion protein. Purified hamster vimentin was readily pulled down with GST-importin-beta, but did not precipitate with GST alone (FIG. 16d). Finally, GST-importin-beta was incubated with axoplasm from different times after lesion, and Western blots of pull-downs were probed for both vimentin and peripherin. As shown in FIG. 16e increasing amounts of vimentin were found in the pull-downs in correlation with time after lesion, whereas peripherin could not be detected at any of the time points examined.

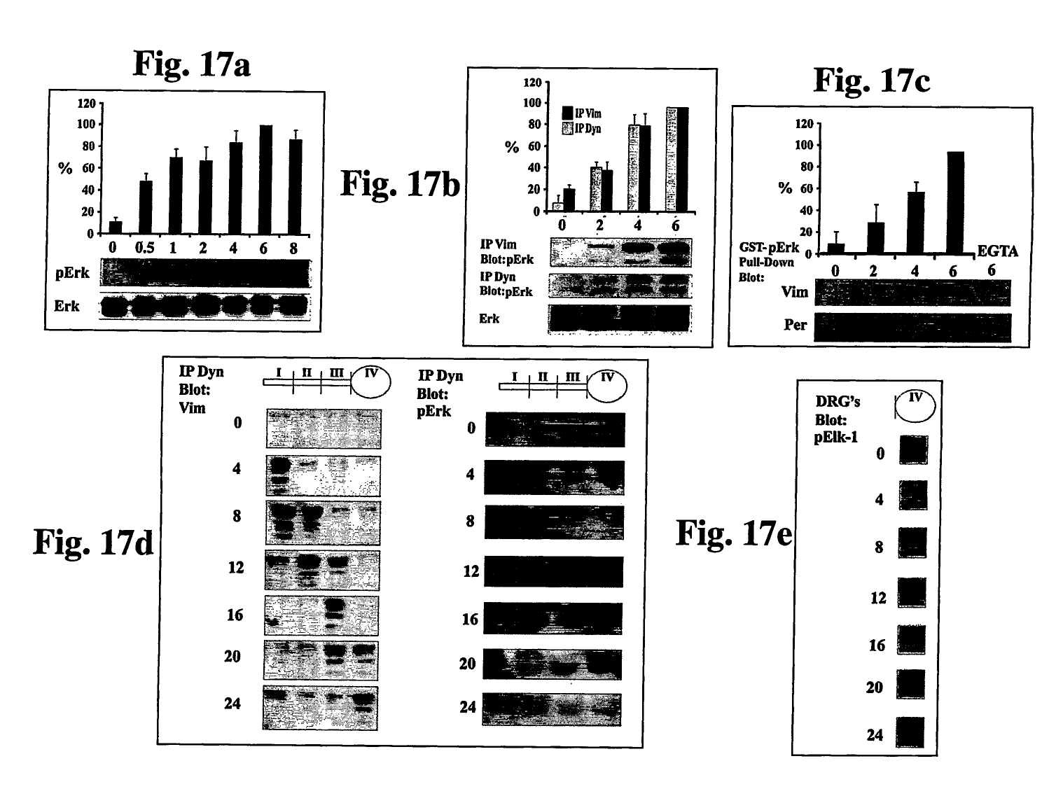

Having established that soluble forms of vimentin interact with the importin-dynein retrograde complex after nerve injury, the role of this intermediate filament molecule in the complex was therefore analyzed. Vimentin has been suggested to act as a scaffold or carrier for signaling molecules (Murakami et al., 2000. Biochim Biophys Acta 1488, 159-166; Paramio and Jorcano, 2002. Bioessays 24, 836-844), or to directly modulate nuclear architecture and/or transcription (Traub, 1995. Physiol Chem Phys Med NMR 27, 377-400). The Aplysia data implicating ERK-like kinases in retrograde injury signaling (Sung et al., 2001. J Neurobiol 47, 67-79), stimulated us to examine the hypothesis that vimentin might act as a scaffold for the retrograde transport of ERKs in mammalian nerve. First, the activation of MAP kinases in lesioned rat sciatic nerve was examined, and phosphorylation of axoplasmic ERK1 and ERK2 within 30 minutes after injury was examined (FIG. 17a). The phosphorylation increased somewhat during the first two hours, and was then maintained stably over at least eight hours after the initial lesion (FIG. 17a). Immunoprecipitation of either vimentin or dynein from axoplasm revealed co-precipitation of the phosphorylated ERKs (pERKs) increasing with time after lesion (FIG. 17b). Incubation of recombinantly produced GST-pERK2 with axoplasm from different time points after lesion revealed co-precipitation of increasing amounts of vimentin, but not peripherin (FIG. 17c). Retrograde transport of vimentin and pERK after sciatic nerve lesion in-vivo was then examined. Crushed nerves were obtained from adult rats at increasing times after lesion, and divided into consecutive segments as shown in FIG. 17d. Axoplasm from each segment was subjected to dynein immunoprecipitation, and precipitates were then analyzed by Western blot for presence of vimentin and pERK. Both molecules moved retrogradely together with dynein over the time course of the experiment, arriving at the L4/L5 DRGs approximately 20 hours after the lesion. Interestingly, vimentin remained associated with dynein up to the 24 hour time point in the DRG, whereas the pERK was apparently dissociated from the complex shortly after arrival in the ganglia (FIG. 17d), thus potentially freeing the MAP kinase for interaction with cytoplasmic or nuclear substrates in the cell body. MAP kinases modulate gene expression by directly phosphorylating transcription factors such as the ETS domain factor Elk-1 (Marais et al., 1993. Cell 73, 381-393; Yang et al., 1999. Embo J 18, 5666-5674). Therefore, Elk-1 phosphorylation in L4/L5 DRG was examined during 24 hours after sciatic nerve lesion in adult rats (FIG. 17e). Two peaks of Elk-1 phosphorylation were observed in DRG lysates, an initial practically immediate event that most likely reflects the response to membrane depolarization events caused by the crush, and a second more sustained phosphorylation that commences upon arrival of pERKs in the ganglia (FIG. 17e).

The discrepancy between dissociation times of vimentin and pERK from the dynein complex upon arrival at the ganglia suggested differential regulation of these two interactions. Nerve injury is known to cause a significant increase in intracellular calcium levels in axons (Ransom and Brown, 2003. Neuron 40, 2-4; Wolf et al., 2001. J Neurosci 21, 1923-1930; Ziv and Spira, 1993. Eur J Neurosci 5, 657-668). This calcium elevation can be of long duration, and recovery kinetics may differ between soma and axon due to the different calcium buffering capacity of these two compartments. The effect of modulating calcium levels on the pERK-vimentin and the vimentin-importin interactions was therefore tested. As shown in FIG. 18a, vimentin binding to pERK2 in-vitro was calcium dependent, and was increased at calcium concentrations of up to 1 millimolar. In contrast, changing calcium concentrations had little or no effect on the vimentin-importin-beta interaction (FIG. 18a). Another possible explanation for the apparent dissociation of pERK in the cell body might be dephosphorylation of the kinase. The effects of increasing concentrations of vimentin on ERK phosphorylation in the presence of alkaline phosphatases (AP) or of sciatic nerve axoplasm (Axp) were therefore tested. Strikingly, vimentin levels in the range of three-fold to six-fold molar ratio of vimentin to pERK provided almost complete protection of pERK2 from the phosphatases, while similar levels of neurofilament had no effect FIG. 18b). The protection of pERK by vimentin was specific, as identical concentrations of vimentin had no effect on hydrolysis of p-nitrophenyl phosphate by alkaline phosphatase (data not shown). The calcium dependence of pERK binding to vimentin prompted us to examine if the phosphatase protection provided by vimentin could also be modulated by calcium. As shown in FIG. 18c, vimentin protected pERK2 from dephosphorylation in a calcium dependent manner, and in a range of calcium concentrations correlated with those allowing maximal pERK-vimentin binding (FIG. 18a).

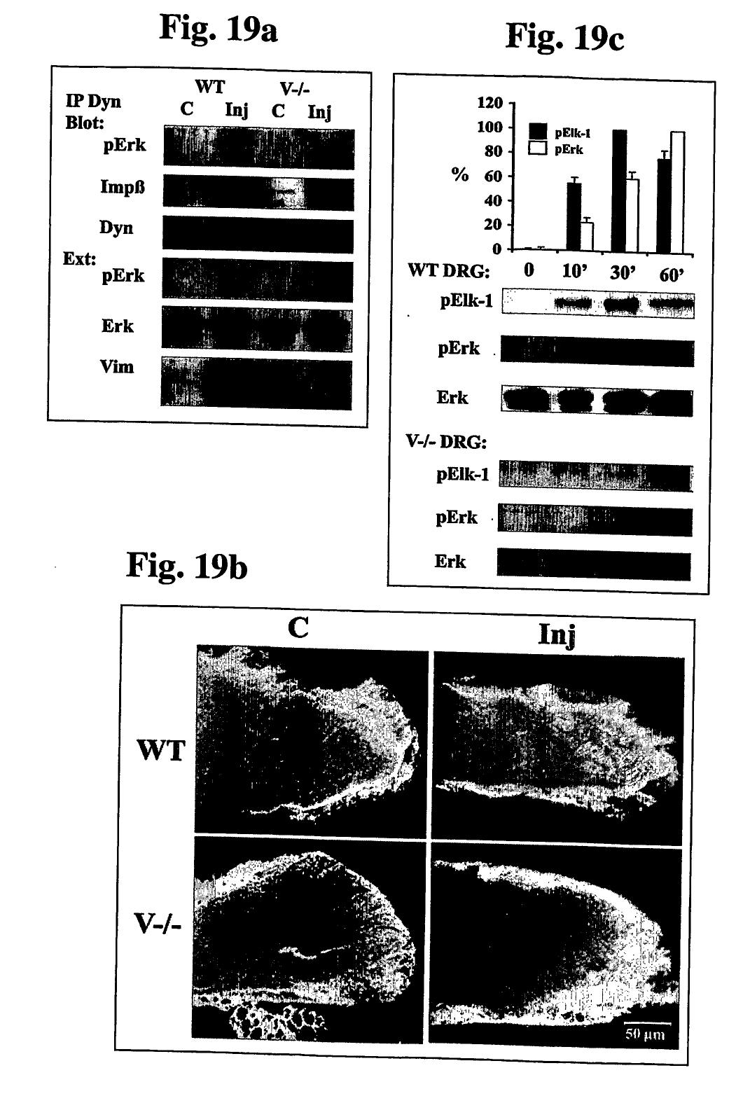

The data described thus far establishes that soluble forms of vimentin interact with the importin-dynein retrograde complex in injured rat sciatic nerve, and thereby transport activated ERKs from the lesion site to activate substrates such as Elk-1 in neuronal cell bodies. In order to obtain further confirmation of these findings, immunoprecipitates of dynein from wild-type and vimentin^{-/-} mouse sciatic nerve axoplasm were compared. The pERKs co-precipitated with dynein in injury axoplasm from wild-type mice, but not from vimentin-nulls (FIG. 19a). Injury-induced ERK phosphorylation was similar in wild-type and vimentin^{-/-} axoplasm, thus the specific difference was the lack of interaction of pERKs with the dynein complex in the vimentin-null background. Retrograde accumulation of pERK in a lesion-ligation paradigm in sciatic nerves of wild-type versus vimentin-null mice was then examined. As shown in FIG. 19b, pERK accumulated at the ligation site in wild-type but not in vimentin^{-/-} sciatic nerve 24 hours after lesion. ERK and Elk-1 phosphorylation were subsequently examined in ganglia from wild-type or vimentin^{-/-} mice, after lesion of the nerve close to the entry points to the DRG (FIG. 19c). In these experiments phosphorylated ERKs appeared in wild-type ganglia from 10 minutes after the crush, peaking at 1 hour post-crush. Elk-1 phosphorylation kinetics was similar to that of the ERKs. In contrast, ganglia from vimentin^{-/-} mice exhibited little or none ERK or Elk-1 phosphorylation over the time course of these experiments (FIG. 19c).

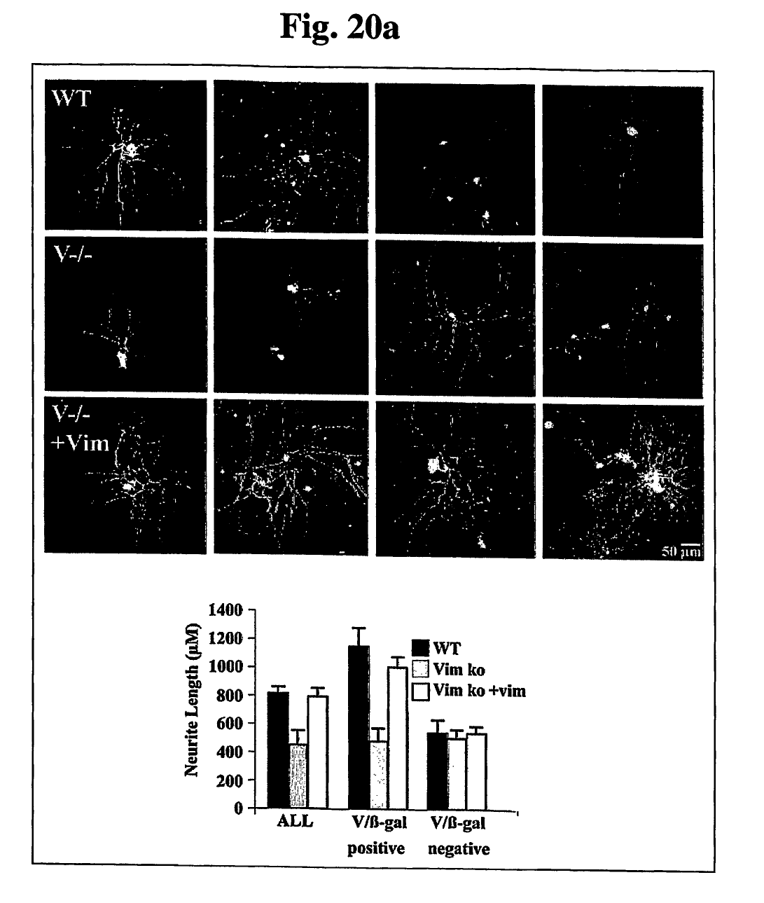

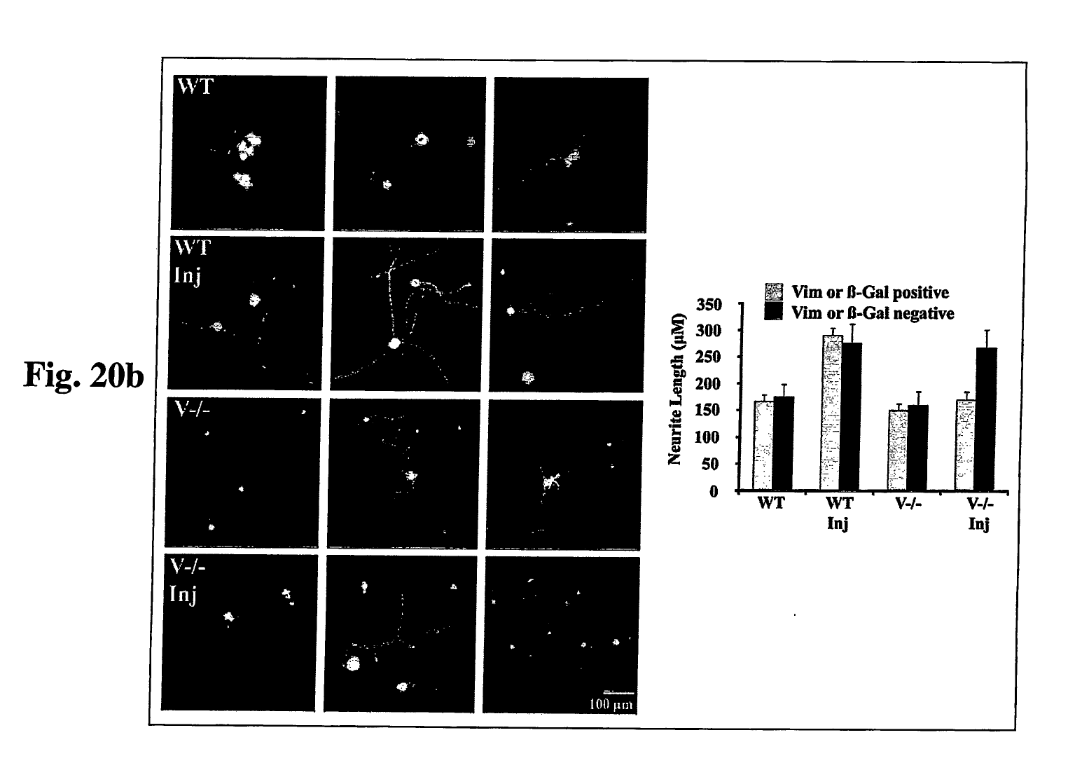

In order to assess the contribution of the vimentin-transported signaling component to the cell body response to injury in-vitro, regenerative outgrowth of adult DRG neurons from vimentin^{-/-} mice was analyzed. Neurite outgrowth from vimentin^{-/-} cells 48 hours in culture was significantly reduced compared to wild-type neurons, and the difference was even more pronounced when comparing only the beta-Gal positive neurons from the knockout to vimentin positive neurons from wild-type mice (FIG. 20a). There was no observable difference in outgrowth capacity of vimentin-null/beta-Gal negative neurons as compared to wild-type/vimentin-negative neurons. Trituration of vimentin-null neurons with calpain-pretreated hamster vimentin rescued the outgrowth deficit in beta-Gal positive cells, and had no effect on neurons that did not express beta-Gal, as shown by comparing outgrowth of beta-Gal-expressing neurons (those originally destined to express vimentin) with similar cells from the same ganglia that did not express beta-Gal.

DRG neurons undergo a transcription-dependent switch from arborizing to long distance neurite outgrowth upon peripheral axotomy by a previous (conditioning) lesion of the sciatic nerve (Smith and Skene, 1997), and this effect is mediated by a retrograde signal (Hanz et al., 2003. Neuron 40, 1095-1104). The conditioning lesion response in DRG of vimentin^{-/-} mice was therefore examined. Sciatic nerve crush was performed three days prior to culture of neurons from the L4/L5 DRGs of vimentin-null and wild-type mice. After 18 hours in culture, pre-lesioned conditioned neurons from wild-type mice extended neurites that were on average two-fold longer than those of naive controls (FIG. 20b). A similar effect was observed in neurons from vimentin-null mice that did not express beta-Gal. In contrast, the vimentin^{-/-}, beta-Gal-positive neurons did not extend longer neurites as a result of the conditioning crash (FIG. 20b). Thus, both in-vitro and in-vivo, the vimentin-mediated signal is required for a complete regenerative response of DRG neurons.

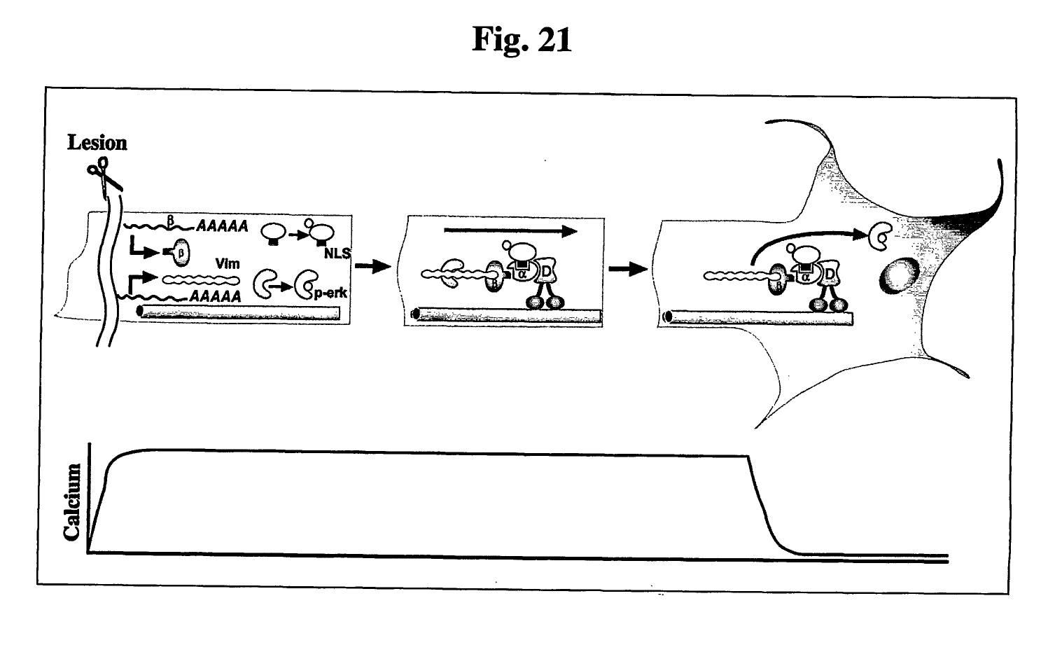

Discussion: The above-described results show that vimentin is upregulated in sciatic nerve axoplasm after lesion and is transported in the retrograde injury-signaling complex via a direct interaction with importin-beta. Strikingly, vimentin binds phosphorylated MAP kinases in a calcium dependent manner, and it was found that the MAP kinases ERK1 and ERK2 are phosphorylated in axoplasm after nerve lesion. Since calcium is elevated in the axon after injury, this provides a tunable switch for docking of activated ERKs to the complex at the lesion site, and their release when the complex arrives in a low calcium compartment (FIG. 21). The pERKs bind to vimentin in a manner that protects them from de-phosphorylation by phosphatases, thus the interaction both links the activated MAP kinase to the retrograde transport machinery and provides a mechanism for preservation of the signaling moiety over long distances. Finally, both injury-induced Elk1 activation and regenerative sprouting are reduced in dorsal root ganglion neurons from vimentin-null mice. Thus, an intermediate filament moiety provides an accessory scaffold for protected retrograde transport of phosphorylated MAP kinases on the importin-dynein complex in lesioned nerve. These data reveal how non-NLS-bearing signaling molecules can access the importins mediated retrograde transport complex, describe a novel role for intermediate filament in the nervous system, and define a mechanism that allows long distance kinase signaling within cells.

A critical initiating event for the mechanism outlined above is local axonal synthesis of both importin-beta and vimentin at the lesion site. Recently it has become apparent that local axonal translation occurs for a wide range of gene products (Giuditta et al., 2002. Trends Neurosci 25, 400-404), contributing to diverse processes of growth cone navigation in the embryonic nervous system (Brittis et al., 2002. Cell 110, 223-235; Campbell and Holt, 2001. Neuron 32, 1013-1026) or regeneration in the adult (Hanz et al., 2003. Neuron 40, 1095-1104; Zheng et al., 2001. J Neurosci 21, 9291-9303). Thus, local translation of specific axonal transcripts provides a versatile mechanism activating latent signaling mechanisms. Indeed local translation might be a prerequisite for vimentin participation in retrograde injury signaling, as anterograde transport of the protein from the cell body may be diverted to filament assembly en route (Prahlad et al., 1998. J Cell Biol 143, 159-170). Vimentin mRNA is known to be targeted to specific subcellular locations in non-neuronal cells (Morris et al., 2000. J Cell Sci 113, 2433-2443; Wiseman et al., 1997. Int J Biochem Cell Biol 29, 1013-1020). In this context it is noteworthy that vimentin synthesis at an axonal lesion places the protein in an environment with elevated calcium levels, thus likely to be unfavorable for filament assembly. High calcium may inhibit filament assembly due to vimentin phosphorylation by calcium-activated CAM kinase II (Inagaki et al., 1997. J Biol Chem 272, 25195-25199), or due to calpain-mediated cleavage of the N-terminal head domain of vimentin (Perides et al., 1987. Eur J Cell Biol 43, 450-458).

The importance of calcium for vimentin availability, pERK binding, and phosphatase protection suggests that the spatiotemporal properties of calcium elevation in the nerve will markedly affect the efficacy of the retrograde signal. Calcium is elevated in axons of both invertebrates and vertebrates after nerve injury or trauma (Strautman et al., 1990. J Neurosci 10, 3564-3575; Stys et al., 1992. J Neurosci 12, 430-439; Ziv and Spira, 1993. Eur J Neurosci 5, 657-668; Ziv and Spira, 1995. J Neurophysiol 74, 2625-2637). Although recovery of axonal calcium to basal levels can be fairly rapid in invertebrate neurons in culture (Ziv and Spira, 1993. Eur J Neurosci 5, 657-668; Ziv and Spira, 1995. J Neurophysiol 74, 2625-2637), levels in injured mammalian nerve may remain elevated for prolonged periods (Howard et al., 1999. Neuroscience 93, 807-815; Iwata et al., 2004. J Neurosci 24, 4605-4613; Wolf et al., 2001. J Neurosci 21, 1923-1930). This might be due to delayed sealing caused by reduced extracellular calcium in the mammalian system (Shi et al., 2000. J Neurophysiol 84, 1763-1769; Yoo et al., 2003. J Neurosci Res 74, 541-551), or due to different rates of sealing in axons of different diameters (Howard et al., 1999. Neuroscience 93, 807-815). Moreover, once a seal is established and calcium levels can begin to recover, the recovery process seems to occur in a gradient along the axon (Ziv and Spira, 1993. Eur J Neurosci 5, 657-668), as has also been observed for calcium elevation due to non-traumatic events along axons (Graf et al., 1999. Dev Neurosci 21, 409-416). Thus, an axonal lesion will lead to significant and prolonged elevation of calcium levels in the axon, thereby facilitating the interaction of pERK with vimentin. If the injury is severe, axonal sealing will be delayed and calcium elevation will be maintained for sufficient time for the pERK signal to arrive at the cell body. If the injury is relatively mild and axon sealing and calcium recovery is rapid, pERK may dissociate before arrival at the cell body; thus the presently disclosed vimentin-pERK mechanism provides a tunable rheostat for conveying information on the extent and severity of the lesion from axon to soma. Conversely, in non-neuronal cells with a significant representation of vimentin in the cytoskeleton the intermediate filament network might act as a “sink’ or anchor for activated ERKs, at least under conditions of local or global increase in calcium levels.

Although the data show that vimentin is required for a normal sprouting response in a subset of DRG neurons, vimentin is not expressed in approximately 40% of the neuronal population, thus other components must be able to substitute or compensate in those cells. As described above, and in the literature (Hanz et al., 2003. Neuron 40, 1095-1104), the present inventors previously showed that retrograde injury signaling can be inhibited by excess NLS peptides, and since NLS peptides did not interfere with the vimentin-pERK interaction, additional signaling molecules are likely transported by the classical importin-alpha NLS-binding site in the complex. Vimentin itself has been reported to bind other signaling molecules such as signaling phospholipase A(2) (Paramio and Jorcano, 2002. Bioessays 24, 836-844), and it is striking that the interaction of cytosolic phospholipase A(2) with vimentin is also calcium sensitive (Murakami et al., 2000. Biochim Biophys Acta 1488, 159-166). Conversely, a different kinase signal may be transported instead of pERKs in vimentin-null cells, for example there is evidence for involvement of the JNK pathway in retrograde injury signaling (Kenney and Kocsis, 1998. J Neurosci 18, 1318-1328). Thus it is becoming apparent that the retrograde injury-signaling complex may traffic a diversity of signals depending on the neuronal subtype and severity of the injury.

Potential diversity in retrograde signals is not the only complicating factor when considering the role of vimentin in nerve regeneration. Integral components of the retrograde injury complex such as importins have other prominent roles in cellular physiology (Weis, 2003. Cell 112, 441-451), and vimentin is after all first and foremost known as a component of cytoskeleton (Evans, 1998. Bioessays 20, 79-86; Fuchs and Cleveland, 1998. Science 279, 514-519; Strelkov et al., 2003. Bioessays 25, 243-251). The initial analyses of vimentin-null mice did not reveal a catastrophic phenotype, and the animals appeared to survive and breed normally (Colucci-Guyon et al., 1994. Cell 79, 679-694). Subsequent analyses have revealed subtle defects in cerebellar glia (Gimenez y Ribotta et al., 2000. Glia 31, 69-83), neurobehavioral effects (Lalonde and Strazielle, 2003. Rev Neurosci 14, 369-385), and impaired skin wound healing due to mesenchymal defects (Eckes et al., 2000. J Cell Sci 113 (Pt 13), 2455-2462), thus vimentin is important in a range of cell types. A recent study has shown reduced astroglial reactivity and increased plastic sprouting of supraspinal axons in hemisected spinal cord of vimentin/GFAP double null mice. This phenotype was attributed to a reduction of the inhospitable environment that would normally be generated by astrocytes at the lesion site (Menet et al., 2003. Proc Natl Acad Sci USA 100, 8999-9004). The outcome of vimentin perturbation in nerve injury will therefore depend on the integration of its “positive” contribution to intrinsic growth mechanisms via retrograde axonal signaling versus its “negative” indirect influence, due to its major structural role in astroglial cells.

The presently described data indicates that a major aspect in the “positive” role of vimentin is the retrograde axonal transport of phosphorylated ERKs. With respect to the contribution of pERK to enhanced regeneration of sensory neurons, Basbaum and colleagues and Filbin and colleagues have reported that elevation of cAMP levels in the DRG mimics the regeneration-inducing effect of a conditioning lesion (Neumann et al., 2002. Neuron 34, 885-893; Qiu et al., 2002. Neuron 34, 895-903). Cross-talk between the cAMP and ERK pathways can occur via inhibition of cAMP phosphodiesterase-4 isoforms by ERK, thus increasing cAMP levels in the cell (Hoffmann et al., 1999. Embo J 18, 893-903; Houslay and Baillie, 2003. Biochem Soc Trans 31, 1186-1190). Indeed it has recently been shown that ERK activated by neurotrophin receptors transiently inhibits cAMP phosphodiesterase-4 in DRG neurons (Gao et al., 2003. J Neurosci 23, 11770-11777). The transient nature of the inhibition may be due to activation of cAMP dependent PKA, which in turn can lead to inhibition of the ERK pathway (Houslay and Baillie, 2003. Biochem Soc Trans 31, 1186-1190; Maeda et al., 2004. Science 304, 875-878). Thus, sustained elevation of cAMP in the cell body may require a prolonged supply of newly phosphorylated ERK, and vimentin mediated trafficking of ERK from the axon could provide such a supply. Thus, direct effects of pERK on transcription factors like Elk-1 may combine with indirect effects via cAMP or other second messengers to coordinate neuronal regeneration.

Finally, and regardless of the role uncovered here for vimentin in axonal transport, the presently disclosed data might explain some hitherto puzzling observations on vimentin movement within cells. Live imaging of cells expressing GFP-vimentin has shown that vimentin is found in complexes of various sizes, which move bi-directionally on microtubules (Ho et al., 1998. J Cell Sci 111 (Pt 13), 1767-1778; Prahlad et al., 1998. J Cell Biol 143, 159-170; Yoon et al., 1998. J Cell Biol 143, 147-157). Conventional kinesin is thought to move vimentin outwards towards plus ends of microtubules (Prahlad et al., 1998. J Cell Biol 143, 159-170) and dynamitin overexpression was recently used to demonstrate a role for dynein in inward minus-end directed movement of vimentin on microtubules (Clarke and Allan, 2002. Curr Biol 12, R596-598; Helfand et al., 2002. J Cell Biol 157, 795-806). However, these findings did not explain why vimentin concentrates in the cell center upon disruption of microtubules (Clarke and Allan, 2002. Curr Biol 12, R596-598; Gurland and Gundersen, 1995. J Cell Biol 131, 1275-1290; Yoon et al., 1998. J Cell Biol 143, 147-157). The direct interaction described above between vimentin and importin-beta provides a molecular link to dynein via the recently described association of importin-alpha with dynein, and suggests that upon microtubule disruption vimentin may concentrate at the cell center due to interactions of importin-beta with nucleoporins. An importin-beta:nucleoporin interaction might also provide a plausible mechanism for vimentin entry to the nucleus, perhaps strengthening the argument for possible nuclear roles of vimentin (Traub, 1995. Physiol Chem Phys Med NMR 27, 377-400; Traub and Shoeman, 1994. Int Rev Cytol 154, 1-103). The presently described findings expand the range of roles and mechanisms for vimentin in the cell, and suggest that intermediate filaments provide dynamic and movable scaffolds for localization and long-distance transport of signaling molecules within cells.

Summary: The above-described results demonstrate that MAP kinases from the ERK family are phosphorylated in sciatic nerve axoplasm upon nerve injury, concomitantly with the appearance of soluble forms of the intermediate filament vimentin in axoplasm. Vimentin binds the phosphorylated ERKs in a calcium dependent manner. This interaction links phosphorylated ERKs to the retrograde transport system via a direct interaction of vimentin with importin-beta. Moreover, vimentin protects phosphorylated ERK from dephosphorylation by phosphatases. Finally, both injury-induced Elk1 activation and sprouting responses are reduced in dorsal root ganglion neurons from vimentin-null mice. Thus, an intermediate filament moiety provides an accessory scaffold for protected retrograde transport of phosphorylated MAP kinases on the importin-dynein complex in lesioned nerve.

As such, the presently described results teach for the first time that regulation of calcium levels can readily be employed for up- or down-regulating attachment of phosphorylated ERKs, and hence of cargo associated with such phosphorylated ERKs, to the retrograde transport machinery. Thus, the presently describe results teach calcium-regulatable retrograde delivery of therapeutic/diagnostic cargo, such as therapeutic/diagnostic agents to inaccessible locations in the central nervous system such as the brain, for instance, for delivering chemotherapeutic agents for diagnosis and treatment, for example, of nervous system malignancies.

### Example 3

### Juxtamembranal, Injury-Independent Expression of the M9 NLS-Binding Importin Family Member Transportin Neurons: Induction of Retrograde Transport of Cargo by Association with Transportin/M9 NLS

As described above, nervous system diseases include numerous highly debilitating and/or lethal diseases, including major diseases, whose pathogenesis is associated with disregulated retrograde transport associated physiological processes in neurons for which no satisfactory treatment and/or diagnostic method is available. An optimal strategy for treating/diagnosing such diseases would be to exploit/regulate neuronal retrograde transport mechanisms to deliver therapeutic/diagnostic compounds to neuronal cell bodies, such as neuronal cell bodies difficult to access or localize. Regulation of retrograde transport mechanisms could also used for optimally regulating physiological processes of neurons generally dependent on retrograde transport, such as growth, survival, and/or differentiation. While various prior art approaches have attempted or suggested methods of exploiting/regulating neuronal retrograde transport mechanisms for achieving such objectives, none have proven to be satisfactory.

While reducing the present invention to practice, the present inventors unexpectedly uncovered, via Western blot analysis, that the importin-beta family member transportin, a nuclear transport factor, is constitutively expressed in nerve axoplasm, and that levels thereof are not regulated by axonal injury (FIG. 23a). The present inventors further unexpectedly uncovered, via fluorescence microscopy analysis, that this molecule is present in a juxtamembrane distribution (FIGS. 23b-c). Transportin is distinct from the classical importin-alpha/-beta pathway in that it binds substrates, such as nuclear ribonucleoprotein A1, which contain the consensus motif of the M9 nuclear localization sequence (NLS; FIG. 22; described in Bogerd et al., 1999. J Biol Chem. 274:9771-7), without requiring importin-alpha. The consensus motif of the M9 NLS is the 12 amino acid residue consensus motif: [Y/F/W]-[X]-[X]-[J]-[X]-[S]-[X]-[Z]-[G]-[P/K]-[M/L/V]-[K/R] (SEQ ID NO: 5), where J is a hydrophilic amino acid residue, Z is a hydrophobic amino acid residue, and X is any residue. The wild-type sequence of the M9 NLS is [Y]-[N]-[N]-[Q]-[S]-[S]-[N]-[F]-[G]-[P]-[M]-[K] (SEQ ID NO: 6). Since transportin is the only protein which is known to directly bind the M9 NLS (Bogerd et al., 1999. J Biol Chem. 274:9771-7), and by virtue of the juxtamembrane localization of transportin in neurons, the present inventors hypothesized that transportin in fact functions as a membrane to nucleus retrograde transporter in axons.

As such, while conceiving the present invention the present inventors hypothesized that compounds such as diagnostic or therapeutic compounds, nucleic acids, viruses, etc., can be attached as cargo to an M9 consensus sequence (SEQ ID NO: 5) or to a transportin molecule and that the resultant conjugates could be delivered to axons so as to induce retrograde transport of such cargo in healthy or injured neurons. Such an approach is particularly advantageous for delivering such cargo to inaccessible locations in the central nervous system such as the brain. Such an approach can be used, for instance, for delivering chemotherapeutic agents via axonal retrograde transport for targeting malignancies with known neurotropisms, especially during metastatic stages of such malignancies. Hence, the presently described method can be used for optimally diagnosing and treating numerous nervous system diseases, overcoming numerous limitations of the prior art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents, patent applications and sequences identified by their accession numbers mentioned in this specification are herein incorporated in their entirety by reference into the specification, to the same extent as if each individual publication, patent, patent application or sequence identified by its accession number was specifically and individually indicated to be incorporated herein by reference. In addition, citation or identification of any reference in this application shall not be construed as an admission that such reference is available as prior art to the present invention.

What is claimed is:

1. A method of inducing retrograde transport of an exogenous compound in an axon, the method comprising:
(a) increasing in the axon an activity and/or a level of a molecule participating in importin mediated retrograde transport; and
(b) administering the exogenous compound to the axon, the exogenous compound being capable of directly or indirectly associating with said molecule participating in importin mediated retrograde transport, thereby inducing retrograde transport of the exogenous compound in the axon.

2. The method of claim 1, wherein step (b) is effected prior to, concomitantly with, or following step (a).

3. The method of claim 1, wherein said increasing in the axon said activity and/or said level of said molecule participating in importin mediated retrograde transport is effected by administering to the axon at least one agent selected from the group consisting of:
(i) an exogenous polynucleotide sequence designed and constructed to express at least a functional portion of said molecule participating in importin mediated retrograde transport;
(ii) a molecule capable of activating said molecule participating in importin mediated retrograde transport; and
(iii) at least a functional portion of said molecule participating in importin mediated retrograde transport.

4. The method of claim 1, wherein said molecule participating in importin mediated retrograde transport is selected from the group consisting of an importin, an intermediate filament protein, a molecule including a nuclear localization signal, and an ERK.

5. The method of claim 4, wherein said importin is importin-beta, transportin or importin-alpha.

6. The method of claim 5, wherein said importin-beta is importin-beta1.

7. The method of claim 5, wherein said importin-alpha is importin-alpha4.

8. The method of claim 4, wherein said intermediate filament protein is a type III intermediate filament protein.

9. The method of claim 8, wherein said type III intermediate filament protein is vimentin or peripherin.

10. The method of claim 4, wherein said nuclear localization signal includes an amino acid sequence set forth in SEQ ID NO: 2 or 5.

11. The method of claim 4, wherein said ERK is selected from the group consisting of ERK1, ERK2 and a phosphorylated ERK.

12. The method of claim 11, wherein said molecule participating in importin mediated retrograde transport is said phosphorylated ERK, and whereas the method further comprises the step of modulating in the axon a level of calcium.

13. The method of claim 1, wherein said molecule participating in importin mediated retrograde transport participates in importin-beta mediated retrograde transport, transportin mediated retrograde transport or importin-alpha mediated retrograde transport.

14. The method of claim 13, wherein said molecule participating in importin-beta mediated retrograde transport participates in importin-beta1 mediated retrograde transport.

15. The method of claim 13, wherein said molecule participating in importin-alpha mediated retrograde transport participates in importin-alpha4 mediated retrograde transport.

16. The method of claim 1, wherein the axon is an injured axon.

17. A method of modulating growth of an axon, the method comprising regulating importin mediated retrograde transport in the axon thereby modulating growth of the axon.

18. The method of claim 17, wherein said regulating importin mediated retrograde transport in the axon is effected by altering in the axon an activity and/or a level of a molecule participating in importin mediated retrograde transport.

19. The method of claim 18, wherein said regulating importin mediated retrograde transport in the axon is up-regulating importin mediated retrograde transport in the axon, and whereas said up-regulating importin mediated retrograde transport in the axon is effected by increasing in the axon said activity and/or said level of said molecule participating in importin mediated retrograde transport.

20. The method of claim 19, wherein said increasing in the axon said activity and/or said level of said molecule participating in importin mediated retrograde transport is effected by administering to the axon at least one agent selected from the group consisting of:
(i) an exogenous polynucleotide sequence designed and constructed to express at least a functional portion of said molecule participating in importin mediated retrograde transport;
(ii) a molecule capable of activating said molecule participating in importin mediated retrograde transport; and
(iii) at least a functional portion said molecule participating in importin mediated retrograde transport.

21. The method of claim 18, wherein said regulating importin mediated retrograde transport in the axon is down-regulating importin mediated retrograde transport in the axon, and whereas said down-regulating importin mediated retrograde transport in the axon is effected by decreasing in the axon said activity and/or said level of said molecule participating in importin mediated retrograde transport.

22. The method of claim 21, wherein said decreasing in the axon said activity and/or said level of said molecule participating in importin mediated retrograde transport is effected by administering to the axon at least one agent selected from the group consisting of:
(a) a molecule capable of binding said molecule participating in importin mediated retrograde transport;
(b) an siRNA molecule capable of inducing degradation of an RNA encoding said molecule participating in importin mediated retrograde transport;
(c) an antisense polynucleotide capable of hybridizing with an mRNA encoding said molecule participating in importin mediated retrograde transport;
(d) a ribozyme capable of cleaving an mRNA encoding said molecule participating in importin mediated retrograde transport; and
(e) a molecule capable of inhibiting ligand-binding of said molecule participating in importin mediated retrograde transport.

23. The method of claim 18, wherein said molecule participating in importin mediated retrograde transport is selected from the group consisting of an importin, an intermediate filament protein, a molecule including a nuclear localization signal, and an ERK.

24. The method of claim 23, wherein said importin is importin-alpha, transportin or importin-beta.

25. The method of claim 24, wherein said importin-beta is importin-beta1.

26. The method of claim 24, wherein said importin-alpha is importin-alpha4.

27. The method of claim 23, wherein said intermediate filament protein is a type III intermediate filament protein.

28. The method of claim 27, wherein said type III intermediate filament protein is vimentin or peripherin.

29. The method of claim 23, wherein said nuclear localization signal includes an amino acid sequence set forth in SEQ ID NO: 2 or 5.

30. The method of claim 23, wherein said ERK is selected from the group consisting of ERK1, ERK2 and a phosphorylated ERK.

31. The method of claim 30, wherein said molecule participating in importin mediated retrograde transport is said phosphorylated ERK, and whereas the method further comprises the step of modulating in the axon a level of calcium.

32. The method of claim 17, wherein said molecule participating in importin mediated retrograde transport participates in importin-beta mediated retrograde transport, transportin mediated retrograde transport or importin-alpha mediated retrograde transport.

33. The method of claim 32, wherein said molecule participating in importin-beta mediated retrograde transport participates in importin-beta1 mediated retrograde transport.

34. The method of claim 32, wherein said molecule participating in importin-alpha mediated retrograde transport participates in importin-alpha4 mediated retrograde transport.

35. The method of claim 17, wherein the axon is an injured axon.

36. A composition-of-matter comprising a compound associated with a molecule participating in importin mediated retrograde transport in an axon, wherein said compound is capable of regulating in a cell a physiological process selected from the group consisting of growth, retrograde transport, survival, and differentiation.

37. The composition-of-matter of claim 36, wherein said molecule participating in importin mediated retrograde transport is selected from the group consisting of an importin, an intermediate filament protein, a molecule including a nuclear localization signal, and an ERK.

38. The composition-of-matter of claim 37, wherein said importin is importin-alpha, transportin or importin-beta.

39. The composition-of-matter of claim 38, wherein said importin-beta is importin-beta1.

40. The composition-of-matter of claim 38, wherein said importin-alpha is importin-alpha4.

41. The composition-of-matter of claim 37, wherein said intermediate filament protein is a type III intermediate filament protein.

42. The composition-of-matter of claim 41, wherein said type III intermediate filament protein is vimentin or peripherin.

43. The composition-of-matter of claim 37, wherein said nuclear localization signal includes an amino acid sequence set forth in SEQ ID NO: 2 or 5.

44. The composition-of-matter of claim 37, wherein said ERK is selected from the group consisting of ERK1, ERK2 and a phosphorylated ERK.

45. The composition-of-matter of claim 44, wherein said molecule participating in importin mediated retrograde transport is said phosphorylated ERK, and whereas the composition-of-matter further comprises calcium.

46. The composition-of-matter of claim 36, wherein said compound is selected from the group consisting of an importin, an intermediate filament protein, a molecule including a nuclear localization signal, and an ERK.

47. The composition-of-matter of claim 46, wherein said importin is importin-alpha, transportin or importin-beta.

48. The composition-of-matter of claim 47, wherein said importin-beta is importin-beta1.

49. The composition-of-matter of claim 47, wherein said importin-alpha is importin-alpha4.

50. The composition-of-matter of claim 46, wherein said intermediate filament protein is a type III intermediate filament protein.

51. The composition-of-matter of claim 50, wherein said type III intermediate filament protein is vimentin or peripherin.

52. The composition-of-matter of claim 46, wherein said nuclear localization signal includes an amino acid sequence set forth in SEQ ID NO: 2 or 5.

53. The composition-of-matter of claim 46, wherein said ERK is selected from the group consisting of ERK1, ERK2 and a phosphorylated ERK.

54. The composition-of-matter of claim 53, wherein said compound is said phosphorylated ERK, and whereas the composition-of-matter further comprises calcium.

55. The composition-of-matter of claim 36, wherein said molecule participating in importin mediated retrograde transport participates in importin-beta mediated retrograde transport, transportin mediated retrograde transport or importin-alpha mediated retrograde transport.

56. The composition-of-matter of claim 55, wherein said molecule participating in importin-beta mediated retrograde transport participates in importin-beta1 mediated retrograde transport.

57. The composition-of-matter of claim 55, wherein said molecule participating in importin-alpha mediated retrograde transport participates in importin-alpha4 mediated retrograde transport.

58. The method of claim 36, wherein said axon is an injured axon.

59. The method of claim 36, wherein said cell is a neuron or a neuron-associated cell.

60. The method of claim 59, wherein said neuron is an injured neuron.

61. A polynucleotide encoding a chimeric polypeptide comprising at least a portion of a molecule participating in importin mediated retrograde transport in an axon, said at least a portion of a molecule participating in importin mediated retrograde transport being fused to an amino acid sequence capable of regulating in a cell a physiological process selected from the group consisting of growth, retrograde transport, survival, and differentiation.

62. The polynucleotide of claim 61, wherein said molecule participating in importin mediated retrograde transport is selected from the group consisting of an importin, an intermediate filament protein, a molecule including a nuclear localization signal, and an ERK.

63. The polynucleotide of claim 62, wherein said importin is importin-alpha, transportin or importin-beta.

64. The polynucleotide of claim 63, wherein said importin-beta is importin-beta1.

65. The polynucleotide of claim 63, wherein said importin-alpha is importin-alpha4.

66. The polynucleotide of claim 62, wherein said intermediate filament protein is a type III intermediate filament protein.

67. The polynucleotide of claim 66, wherein said type III intermediate filament protein is vimentin or peripherin.

68. The polynucleotide of claim 62, wherein said ERK is ERK1 or ERK2.

69. The polynucleotide of claim 62, wherein said nuclear localization signal includes an amino acid sequence set forth in SEQ ID NO: 2 or 5.

70. The polynucleotide of claim 61, wherein said molecule participating in importin mediated retrograde transport participates in importin-beta mediated retrograde transport, transportin mediated retrograde transport or importin-alpha mediated retrograde transport.

71. The polynucleotide of claim 70, wherein said molecule participating in importin-beta mediated retrograde transport participates in importin-beta1 mediated retrograde transport.

72. The polynucleotide of claim 70, wherein said molecule participating in importin-alpha mediated retrograde transport participates in importin-alpha4 mediated retrograde transport.

73. The method of claim 61, wherein said axon is an injured axon.

74. A nucleic acid construct including the polynucleotide of claim 61.

75. A host cell transformed with the nucleic acid construct of claim 74.

76. The cell of claim 75, wherein the cell is a neuron.
